(19) United States
(12) Patent Application Publication
(10) Pub. No.: US 2022/0365091 A1
(43) Pub. Date: Nov. 17, 2022

(54) Intratumoral TFR Cells Curtail Anti-PD-1 Treatment Efficacy

(71) Applicants: LA JOLLA INSTITUTE FOR ALLERGY AND IMMUNOLOGY, La Jolla, CA (US); UNIVERSITY OF SOUTHAMPTON, Southampton (GB)

(72) Inventors: Pandurangan Vijayanand, La Jolla, CA (US); Christian Ottensmeier, La Jolla, CA (US); Simon Eschweiler, La Jolla, CA (US)

(21) Appl. No.: 17/625,226

(22) PCT Filed: Jul. 11, 2020
(86) PCT No.: PCT/US2020/041734

§ 371 Date: Jan. 6, 2022

Related U.S. Application Data
(60) Provisional application No. 62/873,185, filed on Jul. 11, 2019.
(60) Provisional application No. 62/971,603, filed on Feb. 7, 2020.

(51) Int. Cl. G01N 33/574; C12Q 1/6886

(52) U.S. Cl. CPC G01N 33/57492; C12Q 1/6886; C12Q 2600/158; C12Q 2600/106

(57) ABSTRACT

The present invention includes methods of detecting follicular regulatory T cells (T_{FR}) comprising: obtaining a biological sample from a subject and detecting whether T_{FR} are increased in the tumor sample by contacting the biological sample with antibodies that detect CD3⁺CD4⁺ FOXP3⁺BCL6⁺ T cells CD3⁺CD4⁺CXCR5⁺GITR⁺ T cells, or both, when compared to a healthy subject, and detecting the increase of T_{FR} in the tumor sample. The present invention also includes combination therapy that depletes follicular regulatory T cells (T_{FR}) with minimal effect on regulatory T cells (T_{REGS}) to prevent or reduce immune related adverse effects (irAEs).

### CROSS-REFERENCE TO RELATED APPLICATIONS

This application claims priority to U.S. Provisional Patent Application Nos. 62/873,185, filed Jul. 11, 2019, and 62/971,603, filed Feb. 7, 2020, the entire contents of each of which are incorporated herein by reference.

### TECHNICAL FIELD OF THE INVENTION

The present invention relates in general to the field of cancer immunotherapy, and more particularly, to the use of intratumoral T_{FR} cells as a biomarker informing the choice of immune checkpoint blockade therapy. It moreover pertains to the occurrence of immunotherapy-mediated immune related adverse events (irAEs) and minimization thereof.

### BACKGROUND OF THE INVENTION

Without limiting the scope of the invention, its background is described in connection with anti-PD-1 therapy.

An increased density of T regulatory cells (T_{REG}) in tumors has been linked to poor survival outcomes¹. In non-cancer settings, T_{REG} cells have been shown to differentiate into PD-1 expressing follicular regulatory T cells (T_{FR}) that restrain germinal center responses². It was not previously known whether such differentiation also occurs in the tumor microenvironment, and if so, whether such tumor-infiltrating T_{FR} cells are molecularly distinct from T_{REG} cells or are activated by anti-PD1 therapy.

What is needed is to prevent immunosuppression caused by anti-PD-1 therapy and/or reduction of irAE frequency or severity caused by immunotherapy.

### SUMMARY OF THE INVENTION

The present invention pertains to T follicular regulatory cells (T_{FR} cells) and their functional role in cancer. To date, T_{FR} cells have been overlooked in cancer. This is a critical oversight, as these cells account for a substantial proportion of tumor-infiltrating CD4⁺ T cells, and importantly, are highly responsive to immune checkpoint blockade. It was found that T_{FR} cells play a pivotal role in anti-tumor immunity and in determining anti-PD-1 treatment efficacy. The present inventors demonstrate that among tumor-infiltrating lymphocytes, T_{FR} cells express the highest levels of the checkpoint receptor PD-1, making them highly susceptive to anti-PD-1 therapy and in turn lead to an accelerated accumulation of highly suppressive intratumoral T_{FR} cells. Thus, by increasing the abundance and/or activity of intratumoral T_{FR} cells, anti-PD-1 therapy cannot only facilitate, but also dampen anti-tumor immune attack. Moreover, it is shown herein that the efficacy of anti-PD-1 therapy can be improved by depleting T_{FR} cells prior to initiating anti-PD1 treatment (e.g., with anti-IL1R2 antibodies).

Combination therapy utilizing anti-CTLA-4 and nivolumab (anti-PD-1) induces more frequent and severe immune related adverse events (irAEs), thus limiting its use. The inventors have found that selective depletion of T_{FR} cells with novel immunotherapy drugs (e.g., anti-IL1R2) that do not significantly affect or deplete T_{REG} cells, result in fewer and less severe irAEs while maintaining treatment efficacy.

In one embodiment, the present invention includes a method of detecting follicular regulatory T cells (T_{FR}) comprising: obtaining a biological sample from a subject; and detecting whether T_{FR} cells are present or increased in the biological sample by contacting the biological sample with antibodies that detect CD3⁺CD4⁺FOXP3⁺BCL6⁺ T cells, CD3⁺CD4⁺CXCR5⁺GITR⁺ T cells, or both. In one aspect, the biological sample is contacted with antibodies that detect CD3⁺CD4⁺CXCR5⁺FOXP3⁺BCL6⁺ T cells, CD3⁺CD4⁺ CXCR5⁺FOXP3⁺ T cells, or CD3⁺CD4⁺CXCR5⁺ BCL6⁺ GITR⁺ T cells, or any combination thereof. In another aspect, the increase of T_{FR} cells is detected in the biological sample as compared to a healthy subject. In other aspect, the increase of T_{FR} cells is detected in the biological sample as compared to a healthy subject. In another aspect, the method further comprises detecting the presence or a high level of expression of at least one of: PD-1, CTLA-4, 4-1BB, ICOS, Tox, Ki67, or TCF1 on the T_{FR}. In another aspect, the step of detecting is measuring mRNA, protein, or both. In another aspect, the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺ BCL6⁺ GITR⁺ T cells or any combination thereof. In another aspect, the T_{FR} cells are not LIN⁻ CD45⁺CD3⁺CD4⁺CXCR5⁻FOXP3⁺BCL6⁻PD-1⁻ cells. In another aspect, the biological sample is a tumor sample is selected from a colorectal, a melanoma, a lung, a liver, a head and neck, or a breast cancer issue. In another aspect, the tumor sample is obtained from a subject suspected of having an immune reactive adverse effect (IRAE). In another aspect, the T_{FR} cells are PD-1^{high}.

In another embodiment, the present invention includes a method of diagnosing and treating a cancer in a patient, the method comprising the steps of: determining whether the patient has an increase in PD-1 expressing follicular regulatory T (T_{FR}) cells in or about the cancer by: obtaining or having obtained a biological sample from the patient; performing or having performed an assay on the biological sample to determine if the patient has an increase in PD-1 expressing T_{FR} cells, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells or any combination thereof, when compared to a reference level generated for specific tumor types or a healthy patient by: identifying that the patient has an increase in T_{FR} cells that will limit the effectiveness of anti-PD-1 cancer therapy; and if the patient has T_{FR} cells or shows an increase in T_{FR} cells, then internally administering a selective T_{FR} cell depleting therapy to the patient, and if the patient does not have T_{FR} cells, an increase in the T_{FR} cells, or if the T_{FR} cells have been depleted by administering a T_{FR} cell depleting therapy to the patient, then administering anti-PD-1 therapy to the patient in an amount sufficient to treat the cancer, wherein a failure to control cancer growth, or an immune related adverse effects (irAE), is lower following the depletion of FoxP3-expressing regulatory T (T_{REG}) cells and the T_{FR} cells in the patient. In one aspect, the presence of T_{FR} cells is determined in a tumor biopsy. In another aspect, the step of detecting is measuring mRNA, protein, or both. In another aspect, the selective T_{FR} cell depleting therapy is at least one of anti-CTLA-4, anti-IL1R2, anti-4-1BB, anti-ICOS, anti-GITR, anti-OX40, or anti-IL1R2, anti-CCR8 therapy, or other targets specifically expressed or enriched on T_{FR} cells when compared to T_{REG} cells and other T cell populations. In another aspect, the cancer is selected from a colorectal, a melanoma, a lung, a liver, a head and neck, and a breast cancer. In another aspect, the T_{FR} cells express one or more of the following markers: FOXP3, GITR, CTLA-4, 4-1BB, ICOS, Tox, Ki67, and TCF1. In another aspect, the presence of T_{FR} cells is further determined by measuring the expression of one or more genes selected from Tnfrsf1b, Lag3, Tigit, Batf, Illr2, Ccr8, Pdcd1, Tox, CCR8, TNFRSF1B, DUSP14, CLP1. In another aspect, the selective T_{FR} cell depleting therapy does not reduce or eliminate T_{REGS}.

In another embodiment, the present invention includes a method for treating a patient suffering from a cancer susceptible to anti-PD-1 therapy, the method comprising the steps of: determining whether the patient has an increase in PD-1 expressing follicular regulatory T (T_{FR}) cells in or about the cancer, when compared to a reference level generated for specific tumor types or a healthy patient by: obtaining or having obtained a biological sample from the patient; and performing or having performed an assay on the biological sample to determine if the patient has PD-1 expressing T_{FR} cells; and if the patient has the PD-1 expressing T_{FR} cells, then administering a PD-1 expressing T_{FR} depleting therapy to the patient, and if the patient does not have the T_{FR} cells or if the T_{FR} cells have been depleted by administering a selective T_{FR} cell depleting therapy to the patient, then administering anti-PD-1 therapy to the patient in an amount sufficient to treat the cancer susceptible to anti-PD-1 therapy and to reduce immune related adverse effects (irAEs), wherein a risk of failure to control cancer growth is lower following the depletion of the T_{FR} cells In one aspect, the presence of T_{FR} cells is determined from a cancer tissue biopsy. In another aspect, the step of detecting is measuring mRNA, protein, or both. In another aspect, the selective T_{FR} cell depleting therapy is at least one of, but not limited to, anti-IL1R2, anti-OX40, anti-TNFR2, anti-CCR8 antibodies or other targets specifically expressed or enriched on T_{FR} cells when compared to T_{REG} cells and other T cell populations. In another aspect, the selective T_{FR} cells depleting therapy is at least one of an anti-CTLA-4, anti-IL1R2, anti-4-1BB, anti-ICOS, anti-GITR, anti-OX40, or anti-IL1R2, anti-CCR8 depletion therapy. In another aspect, the T_{FR} cells are CD3⁺CD4⁺ FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺ CXCR5⁺FOXP3⁺ T cells, or CD3⁺CD4⁺CXCR5⁺ BCL6⁺ GITR⁺ T cells or any combination thereof. In another aspect, the cancer is selected from a colorectal, a melanoma, a lung, a liver, a head and neck, and a breast cancer. In another aspect, the T_{FR} cells express or have a high level of expression one or more of the following markers: PD-1, BCL6, FOXP3, CXCR5, GITR, CTLA-4, 4-1BB, ICOS, Tox, Ki67, and TCF1. In another aspect, the presence of T_{FR} cells is determined by measuring the expression of two or more genes or proteins selected from Tnfrsf1b, Lag3, Tigit, Batf, Illr2, Ccr8, Pdcd1, Tox, CCR8, TNFRSF1B.

In another embodiment, the present invention includes a method of determining if a patient has follicular regulatory T (T_{FR}) cells that will increase cancer growth, or cause an immune-related adverse effect (irAE), when treated with anti-PD-1 therapy comprising: obtaining a biological sample from a patient; and detecting the T_{FR} cells in the biological sample by contacting the biological sample with antibodies that detect CD3⁺CD4⁺ FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺ CXCR5⁺FOXP3⁺ T cells, or CD3⁺CD4⁺CXCR5⁺ BCL6⁺ GITR⁺ T cells or any combination thereof, when compared to a reference level generated for specific tumor types or a healthy patient, and detecting the T_{FR} cells in the biological sample, wherein if the patient has an increase in T_{FR} cells in the biological sample anti-PD-1 therapy will increase cancer growth or cause the irAE. In one aspect, the method further comprises detecting the presence or a high level of expression of at least one of: GITR, CTLA-4, 4-1BB, ICOS, Tox, Ki67, or TCF1 on the T_{FR} cells. In another aspect, the step of detecting is measuring mRNA, protein, or both. In another aspect, the selective T_{FR} cell depleting therapy is at least one of, but not limited to, anti-IL1R2, anti-OX40, anti-TNFR2, anti-CCR8 antibodies or other targets specifically expressed or enriched on T_{FR} cells when compared to T_{REG} cells and other T cell populations. In another aspect, the T_{FR} cell depleting therapy is at least one of anti-CTLA-4, anti-IL1R2, anti-4-1BB, anti-ICOS, anti-GITR, anti-OX40, or anti-IL1R2, anti-CCR8 depletion therapy. In another aspect, the T_{FR} cells are LIN⁻CD45⁺CD3⁺CD4⁺BCL6⁺ FOXP3⁺PD-1⁺GITR⁺ or LIN⁻CD45⁺CD3⁺CD4⁺CXCR5⁺PD-1⁺GITR⁺. In another aspect, the T_{FR} cells are not LIN⁻CD45⁺CD3⁺CD4⁺CXCR5⁻FOXP3⁺BCL6⁻PD-1⁻ cells. In another aspect, the biological sample is a cancer tissue. In another aspect, the biological sample is selected from a colorectal, a melanoma, a lung, a liver, a head and neck, or a breast cancer tissue. In another aspect, the T_{FR} cells are PD-1^{high}.

In another embodiment, the present invention includes a method of depleting follicular regulatory T cells (T_{FR}) cells without affecting regulatory T (T_{REGS}) cells, comprising: treating a T cell population with a treatment that reduces or eliminates PD-1 expressing T_{FR} cells and co-administering anti-IL1R2 antibodies to protect T_{REGS}, in order to prevent or reduce immune related adverse events (irAEs). In one aspect, the irAE is stimulation of CD4 or CD8 T cell proliferation, T_{FR} cells infiltrating a tumor, or that reduces or abrogates the effectiveness of an anticancer therapy. In another aspect, the anticancer therapy is anti-PD-1 therapy of a cancer selected from colorectal, melanoma, lung, liver, head and neck, or breast cancer. In another aspect, the T_{FR} cells are PD-1^{high}. In another aspect, the selective elimination of T_{FR} cells is in vitro.

In another embodiment, the present invention includes a method of depleting follicular regulatory T cells (T_{FR}) cells without affecting regulatory T (T_{REGS}) cells, comprising: treating a patient with reagents that selectively eliminate (PD-1 expressing) T_{FR} cells without significantly affecting or depleting T_{REG} cells, in order to prevent or reduce the occurrence of immune related adverse events (irAEs). In another aspect, the anticancer therapy is anti-PD-1 therapy of a cancer selected from colorectal, melanoma, lung, liver, head and neck, or breast cancer. In another aspect, the T_{FR} cells are PD-1^{high}.

In another embodiment, the present invention includes a method of reducing immune related adverse events (irAEs) comprising: selectively depleting T_{FR} cells, but not all FOXP3-expressing (T_{regs}, T_{FR}, or both) cells, by specifically targeting T_{FR}-specific cells with at least one of anti-CTLA-4, anti-IL1R2, anti-4-1BB, anti-ICOS, anti-GITR, anti-OX40, or anti-IL1R2, anti-CCR8 depletion. In one aspect, the irAE is stimulation of at least one of CD4 or CD8 T cell proliferation, T_{FR} infiltrating a tumor, or that reduces or abrogates the effectiveness of an anticancer therapy. In another aspect, the anticancer therapy is anti-PD-1 therapy of cancers selected from colorectal, melanoma, lung, liver, head and neck, or breast cancer. In another aspect, the T_{FR} cells are PD-1^{high}.

### BRIEF DESCRIPTION OF THE DRAWINGS

For a more complete understanding of the features and advantages of the present invention, reference is now made to the detailed description of the invention along with the accompanying figures and in which:

FIGS. 1a to 1h show that tumor-infiltrating T_{FR} cells are highly prevalent in human cancers and exhibit features of superior functionality. FIG. 1a, Integrated analysis of 9 single-cell RNA-seq datasets displayed by uniform manifold approximation and projection (UMAP) from 6 different cancer types. Seurat clustering of 25,149 CD4⁺ T cells colored based on cluster type (left panel) and study (middle panel); Right panel shows Seurat-normalized expression of FOXP3 in different clusters (see also Methods). FIG. 1B, FIG. 1c, Bar charts depicting the frequency of FOXP3⁻ and FOXP3⁺ or T_{REG} in tumor-infiltrating CD4⁺ T cells (FIG. 1B), or BCL6⁺ T_{FR}, CXCR5⁺ T_{FR}, BCL6⁺CXCR5⁺ T_{FR} in tumor-infiltrating T_{REG} cells (FIG. 1c) in the assessed datasets. (FIG. 1d) Immunohistochemistry analysis showing proportion of FOXP3⁻ and FOXP3⁺ CD4⁺ cells (upper panel) or T_{REG} and T_{FR} cells (lower panel) in tumor samples from NSCLC patients as in (FIG. 1e-FIG. 1h). Representative immunohistochemistry staining (right plot) for one of the patients is shown, Nuclei (dark blue), FOXP3 (red), CXCR5 (yellow), CD4 (light blue), BCL6 (magenta) and PanCK (green), white arrows characterize CD4⁺FOXP3⁺BCL6⁺CXCR5⁺ T_{FR} cells. FIG. 1e-FIG. 1h, flow cytometric analysis and representative contour plots of CD4⁺ T cells, T_{REG} cells, T_{FR} cells, and histogram plots of CD8⁺ and CD4⁺ TILs from 10 treatment naïve NSCLC patients depicting the frequency of CD8⁺ T cells (blue, LIN⁻CD45⁺CD3⁺CD8⁺), T_{REG} (teal, LIN⁻CD45⁺CD3⁺CD4⁺CXCR5⁻CD127⁻CD25⁺) and T_{FR} (yellow, LIN⁻ CD45⁺CD3⁺CD4⁺CXCR5⁺GITR⁺) cells (FIG. 1e), the frequency and mean fluorescence intensity (MFI) of CD25 and ICOS (FIG. 1f) intracellular CTLA-4 expression in T_{REG} (teal, LIN⁻ CD45⁺CD3⁺CD4⁺CXCR5⁻FOXP3⁺BCL6⁻) and T_{FR} (yellow, LIN⁻CD45⁺CD3⁺CD4⁺BCL6⁺FOXP3⁺) (FIG. 1g), and the frequency and MFI of PD-1 expression (FIG. 1h), grey depicts respective fluorescence minus one (FMO) controls in histogram plots. Data are mean+/−S.E.M.; Wilcoxon matched-pairs signed rank test between T_{REG} and T_{FR} cells.

FIGS. 2a to 2j shows a comparison of human tumor-infiltrating T_{REG} and T_{FR} cells. FIG. 2a, Analysis of 10× single-cell RNA-seq data displayed by manifold approximation and projection (UMAP). Seurat clustering of 8,722 CD4⁺ and CD8⁺ T cells from primary tumor tissue and metastasized tumor-infiltrated lymph nodes colored based on cluster type (left panel), the other three panels are showing Seurat-normalized expression of CD8B, CD4 and FOXP3 respectively. FIG. 2b, Heatmap comparing gene expression of cells in cluster 1 versus cluster 6. Depicted are transcripts that change in expression more than 0.25-fold and adjusted P value of ≤0.05. FIG. 2c, FIG. 2d Gene set enrichment analysis for follicular feature⁴² (c) and T_{FR} feature genes (FIG. 2d), derived from FIG. 2j) for cells in cluster 6 and cluster 1 ordered by Log 2 fold change. e, Ingenuity pathway analysis of differentially expressed transcripts (n=1245) between cluster 1 and cluster 6. f, Violin plots comparing expression levels of indicated transcripts in cluster 1 (left) and cluster 6 (right) cells. FIG. 2g, TraCer plots of all clonally expanded cells (=/>2 clonotypes) in cluster 1 and cluster 6 colored by cluster origin (cluster 1 green, cluster 6 yellow). FIG. 2h, Euler diagram shows overlap between clonotypes in cluster 1 and cluster 6. i FIG. 2, bar chart depicting the mean percentage of clonally expanded cells in cluster 1 and cluster 6. FIG. 2j, Heatmap illustrating the intersection of differentially expressed genes (with mean TPM>25) when comparing 4-1BB⁻ T_{REG} cells with three populations: 4-1BB⁺ T_{REG}, clonally-expanded T_{REG} cells sharing their TCRs with T_{FR} and clonally-expanded T_{FR} cells (distinct cell populations are indicated with colored bars). Genes linked to immunosuppressive function, co-stimulation, and tissue residency are highlighted.

FIGS. 3a to 3j show the frequency and functional responsiveness of T_{FR} cells in murine tumor models. FIG. 3a, Mice were inoculated with B16F10-OVA or MC38-OVA cells subcutaneously (s.c.) on the right flank. Analyses of tumor-infiltrating T_{REG} (Cd19⁻Cd45⁺Cd3⁺Cd4^{+Bcl}6⁻FoxP3⁺) and T_{FR} (Cd19⁻ Cd45⁺Cd3⁺Cd4⁺Bcl6⁺FoxP3⁺) cells were performed at indicated time points. FIG. 3b, Flow-cytometric analysis of the frequency of tumor-infiltrating T_{REG} and T_{FR} cells in indicated tumor models at indicated time points. FIG. 3c, Flow-cytometric analysis of the MFI and frequencies of expression of Ki-67, Tcf1 and 4-1bb in indicated cell types in the B16F10-OVA model at day 14 (n=10). FIG. 3d, Representative FACS plots depicting the expression of Tox (x-axis) and Tcf1 (y-axis) in CD8⁺ T cells, T_{REG} cells and T_{FR} cells, Flow-cytometric analysis of the frequency of Tox-expressing cells in indicated cell types in the B16F10-OVA model at day 14 (n=10). e, Schematic of immunization model in which mice were immunized intraperitoneally (i.p.) with Ovalbumin in alum and treated with an IL-2/anti-IL2R complex at indicated time points for in vivo T_{REG} cell expansion; shown are representative FACS plots characterizing splenic T_{REG} (CD4⁺CXCR5⁻CD25⁺GITR⁺) and T_{FR} (CD4⁺CXCR5⁺CD25⁺GITR⁺) cells and FoxP3 expression validating the gating strategy. FIG. 3f, Contour plots depicting the expression levels of FoxP3 in the indicated cell populations from (FIG. 3e) FIG. 3g, Representative histogram plots depicting the dilution of cell trace violet (CTV) in CD8⁺ T cells with or without addition of T_{REG} or T_{FR} cells. FIG. 3h, Flow-cytometric analysis of an in vitro proliferation assay, depicted is the frequency of proliferating CD8⁺ T cells when co-cultured with different proportions of T_{REG} cells (green) or T_{FR} cells (yellow). FIG. 3i, indicated cells were transferred into B16F10-OVA tumor-bearing RAG1^{-/-} recipient mice at day 3 after tumor inoculation, tumor volume of mice treated as indicated is shown (n=5/group). FIG. 3j, Flow-cytometric analysis depicting the MFI of the expression of indicated markers in indicated cell types in the B16F10-OVA model at day 14. Representative histogram plots are displayed. Data are mean+/−S.E.M.; Significance for comparisons were computed using Mann-Whitney test. All data are representative of two independent experiments.

FIGS. 4a to 4h shows that T_{FR} cells are highly responsive to ICB. FIG. 4a, Mice were s.c. inoculated with B16F10-OVA or MC38-OVA cells and treated with anti-PD-1 Abs at indicated time points. Flow-cytometric analysis of the frequency of tumor-infiltrating T_{REG} and T_{FR} cells, as well as fold induction of both cell types following anti-PD-1 therapy in the B16F10-OVA model (left panel, n=9) and MC38-OVA model (right panel, n=5). FIG. 4b-FIG. 4d, Mice were s.c. inoculated with B16F10-OVA cells and treated with tamoxifen and anti-PD-1 Abs at indicated time points. Tumor volume (FIG. 4b), T_{FR} cell frequencies (FIG. 4c) and eGFP cell frequencies (FIG. 4d) for indicated mice are shown, n=6-8 mice/group. FIG. 4e, Mice were immunized i.p. with Ovalbumin in alum and additionally treated with an IL-2/anti-IL-2R complex at indicated time points. OT-I CD8⁺ T cells, GFP⁺ and YFP⁺ T_{REG} cells were adoptively transferred into B16F10-OVA tumor-bearing Rag1^{-/-} mice at day 3 after tumor inoculation. Cell frequencies of eGFP and YFP in spleen (left) or tumor tissue (right) are shown, n=7. FIG. 4f, Flow-cytometric analysis of Bcl6 expression in splenic CD4⁺FoxP3⁺ cells of FoxP3^{YFP-cre}×Bcl-6^{fl/fl} mice (grey), FoxP3^{eGFP} mice (blue) and tumor-infiltrating CD4⁺FoxP3⁺ cells (red) 13 days after adoptive transfer into B16F10-OVA tumor-bearing Rag1^{-/-} mice. FIG. 4g, FIG. 4h, Survival curves of an independent cohort of melanoma patients (n=29) stratified into T_{FR}^{hi} (>5.075% of CD4⁺ cells co-expressing FOXP3 and BCL6) and T_{FR}^{lo} (<5.075% of cells co-expressing FOXP3 and BCL6) (g), Survival curves of melanoma patients stratified into CXCR5^{hi} (frequency of CXCR5+ cells >8.336%) and CXCR5^{lo} (frequency of CXCR5+ cells <8.336%) (FIG. 4h). Data are mean+/−S.E.M., Significance for comparisons were computed using Mann-Whitney test (FIG. 4a-FIG. 4e) or Mantel-Cox test (FIG. 4g, FIG. 4h). Data in FIG. 4a-FIG. 4e are representative of two independent experiments.

FIGS. 5a to 5h shows the clinical benefit of sequential ICB. FIG. 5a-FIG. 5c, Mice were s.c. inoculated with B16F10-OVA cells and treated with anti-CTLA-4 and/or anti-PD-1 Abs at indicated time points, Tumor volume (FIG. 5b) and cell frequencies (FIG. 5c) of mice treated as indicated in (FIG. 5a), n=8-13 mice/group. FIG. 5d, Summary of clinical features of an independent cohort of melanoma patients (n=271) stratified into 5 groups based on ICB treatment regimen. FIG. 5e, Survival curves of patient cohort (FIG. 5d). FIG. 5f-FIG. 5h, Survival curves for early onset disease (FIG. 5f) (M1a and M1b combined), late stage disease (FIG. 5g) (M1c and M1d combined) or BRAF mutation status (FIG. 5h) of patient cohort (FIG. 5c). One-way ANOVA was used to compare the mean of each column with the mean of each other column followed by Dunnett's test (FIG. 5b, FIG. 5c) or Mantel-Cox test (FIG. 5f-FIG. 5h). Data in FIG. 5b, FIG. 5c are representative of two independent experiments.

FIGS. 6a to 6c shows the selection criteria for the integrated single-cell analysis and gating strategies. FIG. 6a, Violin plots depicting single-cell expression levels for BCL6, CXCR5 and FOXP3 transcripts (left panel) in tumor-infiltrating CD4⁺ T cells of an exemplary dataset⁶⁰; dotted lines indicate threshold used for defining positive cells. The scatter plot (right panel) shows expression levels of BCL6 and CXCR5 transcripts in FOXP3-expressing CD4⁺ T cells FIG. 6b, Gating strategy (surface panel) to sort tumor-infiltrating T_{REG} (LIN⁻CD45⁺CD3⁺CD4⁺CXCR5⁻CD127⁻CD25⁺) and T_{FR} (LIN⁻ CD45⁺CD3⁺CD4⁺CXCR5⁺GITR⁺) cells is shown in the representative FACS plots. FIG. 6c, Gating strategy (intracellular panel) to identify tumor-infiltrating T_{REG} (LIN⁻CD45⁺CD3⁺CD4⁺CXCR5 FOXP3⁺BCL6⁻) and T_{FR} (LIN⁻CD45⁺CD3⁺CD4⁺BCL6⁺FOXP3⁺) cells is shown in the representative FACS plots.

FIGS. 7a to 7f shows the transcriptome analysis of murine T_{FR} cells and characterization of T_{FR} cells in murine tumors. FIG. 7a, Schematic of immunization model in which mice were immunized intraperitoneally (i.p.) with Ovalbumin in complete Freund's adjuvant, Ovalbumin in Monophosphoryl Lipid A or mock PBS. FIG. 7b, tSNE plot of T_{EFF} (Cd19⁻Cd45⁺Cd3⁺Cd4⁺Cxcr5⁻Gitr⁻Cd25⁻Cd62L⁻ Cd44⁺), T_{REG} (Cd19⁻Cd45⁺Cd3⁺Cd4⁺Cxcr5⁻Gitr⁺Cd25⁺), T_{FH} (Cd19⁻Cd45⁺Cd3⁺Cd4⁺Cxcr5⁺Gitr⁻) and T_{FR} (Cd19⁻Cd45⁺Cd3⁺Cd4⁺Cxcr5⁺Gitr⁺). Each symbol represents an individual mouse sample (n=9 for T_{EFF}, n=11 for T_{REG}, n=11 for T_{FH}, n=11 for T_{FR}) that passed quality controls. FIG. 7c, Euler diagrams show the overlap of differentially expressed genes (left, upregulated in T_{FR}, right, downregulated in T_{FR}) in T_{FR} cells compared to the indicated cell types. FIG. 7d, Heatmap comparing gene signatures of T_{EFF}, T_{REG}, T_{FH} and T_{FR} cells. Depicted are transcripts that change in expression more than 2-fold with a DEseq2 adjusted P value of ≤0.05. FIG. 7e, Log transformed RNA-seq expression values for each of the indicated differentially expressed genes. Each symbol represents an individual sample, data are mean+/−S.E.M. FIG. 7f, Representative histogram plot showing MFI of the surface expression of indicated markers in human tumor-infiltrating T_{FR} cells (n=4).

FIGS. 8a to 8g shows the transcriptome analysis of human tumor-infiltrating T_{FR} cells. FIG. 8a, Weighted gene co-expression network analysis (WGCNA) depicted as a Topological Overlap Matrix (TOM) heatmap. It included all genes used in the WGCNA analysis and each row and column correspond to a single gene. Red color indicates the degree of topological overlap. The signed network was generated with bulk RNA-seq data of sorted cells enriched for tumor-infiltrating T_{REG} (LIN CD45⁺CD3⁺CD4⁺CXCR5⁻CD127⁻CD25⁺) and T_{FR} (LIN⁻CD45⁺CD3⁺CD4⁺CXCR5⁺GITR⁺) populations respectively from 10 treatment naïve NSCLC patients (as described in FIG. 1d— FIG. 1g). FIG. 8b, Spearman correlation analysis of the modules identified in (FIG. 8a), depicting module correlation with T_{FR} phenotype. Genes in the pink module are visualized in Gephi, BCL6 and FOXP3 are highlighted. c, Ingenuity pathway analysis of genes in pink module (FIG. 8b). Shown are the top 5 canonical pathways ordered by P value. FIG. 8d, flow cytometric analysis of the frequency (upper panel) and MFI (lower panel) of Ki67-expressing cells, representative histogram plots (right panel) for tumor-infiltrating CD8⁺ T cells, T_{REG} and T_{FR} cells (n=10) from patient samples (described in FIG. 1e-FIG. 1f). FIG. 8e, Heatmap comparing gene expression signatures of enriched population of tumor-infiltrating T_{REG} cells (green) and T_{FR} cells (yellow). Depicted are transcripts that change in expression more than 2-fold with an adjusted P value of ≤0.05. FIG. 8f, Weighted gene co-expression network analysis visualized in Gephi, the nodes are colored and sized according to the number of edges (connections), and the edge thickness is proportional to the edge weight (strength of correlation). The top 10 most differentially expressed genes between T_{REG} and T_{FR} cells are highlighted. FIG. 8g, flow cytometric analysis of the frequency of tumor-infiltrating TCF1⁺ T_{REG} and T_{FR} cells (n=5). Data are mean+/−S.E.M. Significance for comparisons were computed using Mann-Whitney test

FIGS. 9a and 9b shows cell trajectory analysis of human T_{REG} and T_{FR} cells from primary tumor tissue and metathesized tumor-infiltrated lymph nodes. FIG. 9a, Single-cell pseudo-time trajectory of cells in cluster 1 (T_{REG} cells) and cluster 6 (T_{FR} cells) (left) or cells from primary tumor tissue or metastatic tumor-infiltrated lymph nodes (right) constructed using the Monocle3 algorithm. FIG. 9b, Normalized gene expression of IL1R2, CCR8, TNFRSF9, TNFRSF18 and PDCD1 on pseudotime path as in (FIG. 9a).

FIGS. 10a to 10e. TCR-seq analysis of tumor-infiltrating T_{REG} and T_{FR} cells. FIG. 10a, the pie chart illustrates the mean percentage of T_{FR} clonotypes that were shared with T_{REG} cells (light blue) and non-T_{REG} cells (grey) respectively, from 4 patients with the highest numbers of clonally expanded FOXP3-expressing cells from a published single cell RNA-seq dataset¹⁷. The lower panel plot displays the percentage of T_{FR} clonotypes that overlap with 4-1BB⁻ or 4-1BB⁺ tumor-infiltrating T_{REG} cells. FIG. 10b, Representative TraCer plot of patient 1010¹⁷ depicting all clonally expanded cells, color indicates the type of tumor-infiltrating CD4⁺ T cells: non-T_{REG} (grey, FOXP3⁻), 4-1BB⁻ T_{REG} (green), 4-1BB⁺ T_{REG} (red) and T_{FR} (yellow) cells. FIG. 10c, Single-cell pseudo-time trajectory of 4-1BB⁻, 4-1BB⁺ T_{REG}, clonally-expanded, TCR-sharing T_{REG} and T_{FR} cells (indicated with colored circles) constructed using the Monocle3 algorithm. FIG. 10d, Correlation of Monocle component 1 (x-axis) with the genes commonly unregulated in 4-1BB⁺ T_{REG}, clonally-expanded, TCR-sharing T_{REG} and T_{FR} cells compared to 4-1BB⁻ T_{REG} cells (y-axis). The solid line represents LOESS fitting between the shared signature and Monocle component 1. FIG. 10e, flow cytometric analysis of the frequency (left panel), MFI (middle panel) and representative histogram plots (right panel) for 4-1BB expression in tumor-infiltrating CD8⁺ T cells, T_{REG} and T_{FR} cells (n=10). Data are mean+/−S.E.M. Significance for comparisons were computed using Mann-Whitney test

FIG. 11 shows the characterization of murine T_{FR} cells in immunization and cancer setting. Gating strategy to identify tumor-infiltrating T_{REG} (Cd19⁻Cd45⁺Cd3⁺Cd4⁺Bcl6⁻FoxP3⁺) and T_{FR} (Cd19⁻ Cd45⁺Cd3⁺Cd4⁺Bcl6⁺FoxP3⁺) cells in Bcl6F10-OVA inoculated mice at d21 (upper panel), shown are representative FACS plots. The FACS plots in the lower panel illustrate intracellular expression of Bcl6 in the indicated cell types (left panel), expression of Gitr (middle upper panel), Ki67 (right upper panel), Pd-1 (middle lower panel), and Ctla4 (right lower panel) versus FoxP3 in Cd4⁺ T cells.

FIGS. 12a to 12e shows human T_{FR} cells are responsive to anti-PD-1 therapy. FIG. 12a, Heatmap comparing gene signatures of human tumor-infiltrating T_{FR} cells pre- (n=21) and post- (n=26) anti-PD-1 therapy¹⁷. T_{FR} cells from 5 patients (P2, P3, P12, P15, P20) receiving anti-PD-1 monotherapy were combined. IPA analysis of transcripts (n=98) more highly expressed post anti-PD-1 treatment (right upper panel) and transcripts that overlap with CD28 signaling, ICOS-ICOSL signaling and T cell receptor signaling are highlighted (right lower panel and heatmap). FIG. 12b, Flow-cytometric analysis of the frequency of tumor-infiltrating T_{REG} and T_{FR} cells in indicated tumor models at indicated time points FIG. 12c, FIG. 12d, IHC analysis of the frequency of FOXP3⁺BCL6⁺ T_{FR} cells (FIG. 12c) with a cutoff (orange line) set to upper limit of normal of 5.075% pertaining to (FIG. 4g), and CXCR5+ cells (FIG. 12d) with a cutoff (orange line) set to upper limit of normal of 8.375% pertaining to (FIG. 4h). FIG. 12e, Mice were s.c. inoculated with B16F10-OVA or MC38-OVA cells and treated with anti-CTLA-4 Abs at indicated time points. Flow-cytometric analysis of the frequency of tumor-infiltrating T_{REG} and T_{FR} cells, as well as fold depletion of both cell types following anti-CTLA-4 therapy in the B16F10-OVA model (left panel, n=9) and MC38-OVA model (right panel, n=5). Data in FIG. 12b-FIG. 12e are mean+/−S.E.M. Data in FIG. 12b, FIG. 12e are representative of two independent experiments.

FIGS. 13A-13C shows survival curves of melanoma patient cohort (n=271) for all patients (left), survival curves for early onset disease (middle) (M1a and M1b combined), late stage disease (right) (M1c and M1d combined) using the present invention.

FIG. 14 shows CRISPR-based deletion of target gene (ICOS) in primary CD4+ T cells. ICOS protein expression following ICOS CRISPRi guide RNA (yellow color) or control guide RNA (blue color) transduction in CD4+ T cells.

FIGS. 15a and 15e show Control mice or FoxP3^{YFPcre}×Bcl6^{fl/fl} (T_{FR} ko) mice were s.c. inoculated with B16F10-OVA cells and treated with isotype control or anti-PD-1 Abs at indicated time points, Tumor volume (FIG. 15a) and cell frequencies (FIG. 15b) of mice treated as indicated n=9-11 mice/group (FIG. 15a) and n=5-7 mice/group (FIG. 15b). FIGS. 15c to 15 e area graphs with the results. One-way ANOVA was used to compare the mean of each column with the mean of each other column followed by Dunnett's test.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

Appended hereto in electronic format only, and as an additional part of the disclosure, are submitted two supplemental Tables 1 and 2 that are an integral part of the disclosure.

As used herein, the terms “treatment”, “treating”, and the like, may include amelioration or elimination of a developed disease or condition once it has been established or alleviation of the characteristic symptoms of such disease or condition, specifically, immune suppression by follicular regulatory T cells (T_{FR}). Specifically, the treatment of a disease or condition that would benefit from preventing excessive activation or accumulation of PD-1 expressing follicular regulatory T cells (T_{FR}), e.g., anti-PD1 treatment in cancer. The treatment would also target the indiscriminate depletion of FoxP3-expressing (T_{REG}+T_{FR}) cells, which results in immune related adverse events (irAEs). As used herein, these terms “treatment”, “treating”, and the like, may also encompass, depending on the condition of the subject, preventing the onset of a disease or condition or of symptoms associated with the disease or condition, including, for example, reducing the immune suppression caused by T_{FR} cells of cytotoxic tumor infiltrating lymphocytes (TIL). Such prevention or reduction prior to affliction may refer to administration of a therapeutic compound to a subject that is not at the time of administration afflicted with the disease or condition.

As used herein, the term “preventing” refers to preventing the indiscriminate depletion of FoxP3-expressing (T_{REG}+T_{FR}) cells, which causes irAEs. Selectively depleting T_{FR} cells (without or with minimal depletion of T_{REG} cells) minimizes irAEs while maintaining treatment efficacy, especially in combination with anti-PD-1 therapy.

As used herein, these terms “subject” or “patient” can be any mammal, including a human and the treatment can be provided in vivo or cells can be treated in vitro and then returned to the subject or patient.

As used herein, the terms “standard control” “control” or “control biological sample” refers to a sample, measurement, or value that serves as a reference, usually a known reference, for comparison to a subject biological sample, test sample, measurement, or value. For example, a test biological sample can be taken from a patient suspected of having a cancer or through the generation of a reference level for specific tumor types or immune related adverse events (irAEs). A standard control can represent an average measurement or value gathered from a T_{FR} population of similar individuals that do not have a given disease or condition (i.e., standard control population), e.g., healthy individuals with a similar medical background, same age, weight, etc. that do not have a cancer or immune related adverse events (irAEs). As shown herein, irAEs are caused by immunotherapy-mediated depletion (e.g., CTLA-4 antibodies) of suppressive FoxP3-expressing cells in multiple tissues (not only cancer tissue). Thus, specifically targeting and depleting T_{FR} cells decreases irAEs while maintaining treatment efficacy. A standard control value can also be obtained from the same individual, e.g., from an earlier-obtained sample, prior to disease or condition (e.g., a cancer or immune related adverse events (irAEs)), or prior to treatment. One of skill in the art will understand which standard controls are valuable in a given situation and be able to analyze data based on comparisons to standard control values. Standard controls are also valuable for determining the significance of data. For example, if values for a given parameter are widely variant in standard controls, variation in test samples will not be considered as significant.

As used herein, the term “T_{FR} cell population” refers to a cell population which has been processed so as to identify the cell population from other cell populations with which it is normally associated in its naturally occurring state using the various markers described herein, including both cell surface markers, but also the expression of genes or proteins that remain intracellularly and can be measures in vivo or ex vivo. The purified T_{FR} cell population can, thus, represent an enriched cell population in that the relative concentration of the cell population in a sample can be increased following such processing in comparison to its natural state. Alternatively, the T_{FR} cell population can be reduced by at least 50%, 60%, 70%, 80%, or at least 90%, or at least 95% or 100% in comparison to its natural state (i.e. pre-treatment) to prevent their immune suppressive activity. Such purified cell population may, thus, represent a cell preparation which can be further processed so as to obtain commercially viable preparations.

Agents for reducing or eliminating T_{FR} cells may be processed so as to be part of a pharmaceutical composition, such as those taught herein. Non-limiting examples include anti-CTLA-4, anti-IL1R2, anti-4-1BB, anti-TNFR2, anti-ICOS, anti-GITR, anti-OX40, and/or anti-CCR8, that lead to T_{FR} depletion. For example, in one embodiment, the cell preparation can be prepared for transportation or storage in a serum-based solution containing necessary additives, which can then be stored or transported in a frozen form. In doing so, the person of skill will readily understand that the cell preparation is in a composition that includes a suitable carrier, which composition is significantly different from the natural occurring separate elements.

For example, the serum-based preparation may comprise human serum or fetal bovine serum, which is a structural form that is markedly different from the form of the naturally occurring elements of the preparation. The resulting preparation includes cells that are in dormant state, for example, that may have slowed-down or stopped intracellular metabolic reactions and/or that may have structural modifications to their cellular membranes. The resulting preparation includes cells that can, thus, be packaged or shipped while minimizing cell loss which would otherwise occur with the naturally occurring cells. This property of minimizing cell loss of the resulting preparation/composition is markedly different from properties of the cells by themselves in nature. A person skilled in the art would be able to determine a suitable preparation without departing from the present disclosure.

As used herein, the term “carrier” refers to any carrier, diluent or excipient that is compatible with the herein described composition that reduces or eliminates T_{FR}, such as, anti-CTLA-4, anti-IL1R2, anti-4-1BB, anti-ICOS, anti-GITR, anti-OX40, and/or anti-CCR8 (including antibodies) that cause T_{FR} depletion or inactivation. Suitable acceptable carriers known in the art include, but are not limited to, water, saline, glucose, dextrose, buffered solutions, and the like. Such a carrier is advantageously non-toxic or has a limited effect on non-T_{FR} immune cells and not harmful to the subject. It may also be biodegradable. The carrier may be a solid or liquid acceptable carrier. A suitable solid acceptable carrier is a non-toxic carrier. For instance, this solid acceptable carrier may be a common solid micronized injectable such as the component of a typical injectable composition for example, but without being limited to, kaolin, talc, calcium carbonate, chitosan, starch, lactose, and the like. A suitable liquid acceptable carrier may be, for example, water, saline, DMSO, culture medium such as DMEM, and the like. The person skilled in the art will be able to determine a suitable acceptable carrier for a specific application without departing from the present disclosure.

As used herein, the terms “determining,” “measuring,” “evaluating,” “assessing,” and “assaying,” as used herein, generally refer to any form of measurement, and include determining if T_{FR} cells are present or not in a biological sample. In addition, these can also be used to determine the abundance of T_{FR} cells. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

As used herein, the term “therapeutically effective amount” may include the amount necessary to allow the component or composition that prevent the T_{FR} cells from performing their immunological role without causing overly negative effects in the host to which the component or composition is administered. In one example, the agents reduce or eliminate T_{FR} cells (or their activity), but do not significantly affect or deplete T_{REG} cells. The exact amount of the components to be used or the composition to be administered will vary according to factors such as the type of condition being treated, the type and age of the subject to be treated, the mode of administration, as well as the other ingredients in the composition.

As used herein, the term “expression” refers to a level of expression of a gene or a protein, that is transcribed from the gene. Generally, an “expression” level is determined by measuring the expression level of a gene of interest for a given cell population, determining the median expression level of that gene for the cell population, and comparing the expression level of the same gene for a particular cell to the median expression level for a different cell population. For example, if the expression level of a gene of interest for the single cell population is determined to be above the median expression level of the patient population, that cell is determined to have high expression of the gene of interest. Alternatively, if the expression level of a gene of interest for the cell population is determined to be below the median expression level of a normal cell population, that cell is determined to have low expression of the gene of interest.

As used herein when referring to a cell surface or other detectable marker, the terms “high” or “high expression” refers to a statistically significant increase in expression compared to naïve T cells. In certain embodiments, a statistically significant increase in expression refers to at least one log higher expression when compared to naïve T cells; in other cases, it can be 2 or even 3 logs higher. The expression can be measured with any number of methods, for example, fluorescence activated cell sorting, RNA-expression, luminescent or chemiluminescent platforms, Illumina®, MesoScale®, or other similar systems. As used herein, for example, when referring to the surface expression of PD-1^{high} in the context of T cells (e.g., T_{FR}), are T cells that have a statistically significant increased expression when compared to naïve T cells. This statistically significant increase refers to, in certain embodiments, at least one log higher surface expression.

### Example 1. T_{FR} Cells Inhibit Anti-Tumor Immunity and are Responsive to Immune Checkpoint Blockade

An increased density of regulatory T cells (T_{REG}) in tumors has been linked to poor survival outcomes¹. In non-cancer settings, T_{REG} cells have been shown to differentiate into PD-1 expressing follicular regulatory T cells (T_{FR}) that restrain germinal center responses². It is not known whether such differentiation also occurs in the tumor microenvironment, and if so, whether such tumor-infiltrating T_{FR} cells are molecularly distinct from T_{REG} cells or are activated by anti-PD1 therapy. In this example, the inventors show that T_{FR} cells are present in high numbers in human and murine tumor tissues, share T cell receptor (TCR) clonotypes with intratumoral T_{REG} cells and express high levels of PD-1. Single-cell TCR data, trajectory analyses and adoptive transfer studies indicate intratumoral conversion of T_{REG} to T_{FR} cells. When compared to T_{REG} cells, T_{FR} cells exhibited enhanced suppressive capacity in vitro and in vivo and expressed higher levels of molecules known to be linked to co-stimulation (4-1BB, ICOS, GITR), cell proliferation (Ki67), suppressive function (CTLA-4), and self-renewal potential (TCF1), all features suggestive of superior functional properties.

In syngeneic tumor models, anti-PD-1 treatment increased the number of tumor-infiltrating T_{FR} cells. Conditional knockout of T_{FR} cells or depletion of T_{FR} cells with anti-CTLA4 antibody prior to anti-PD1 treatment, improved tumor control in mice. Notably, in a cohort of 271 melanoma patients, treatment with anti-CTLA-4 followed by anti-PD-1 at progression was associated with better long-term survival outcomes than anti-PD-1 or anti-CTLA-4 monotherapy, anti-PD-1 followed by anti CTLA-4 at progression or concomitant combination therapy. These findings illustrate that anti-PD1 therapy has the potential to regulate abundance and/or functionality of T_{FR} cells, and can thus induce a profoundly immunosuppressive milieu impeding anti-tumor immunity. Thus, indiscriminate use of anti-PD-1 therapy can prove detrimental in some patients.

Follicular regulatory T cells (T_{FR}) inhibit T and B cell responses to mitigate germinal center reactions in secondary lymphoid organs³, impede humoral immunity towards self-antigens² and display heightened suppressive capacity when compared to T_{REG} cells^{4,5}. T_{FR} cells are being characterized by their joint expression of the surface molecules Cxcr5 and Gitr^{2,6}, or by their co-expression of the transcription factors FoxP3 and Bcl6⁷. Several studies have demonstrated that, depending on disease context and organ, cells of the T follicular lineage express varying levels of CXCR5 and BCL6^{8,9}. Moreover, it has been shown that deletion of Cxcr5 expression in FoxP3-expressing cells does not abrogate the development and maintenance of Bcl6⁺ T_{FR} cells⁷, indicating that distinct subsets of T_{FR} cell cells exist, which not only differ in their expression of Cxcr5 and Bcl6, but also in their expression of CD25^{10,11}.

Most tumors contain tertiary lymphoid structures and because cancerous cells frequently express self or altered-self antigens, the inventors investigated if T_{REG} and T_{FR} cells accumulate in parallel in the tumor microenvironment (TME) as a means of effective immune evasion.

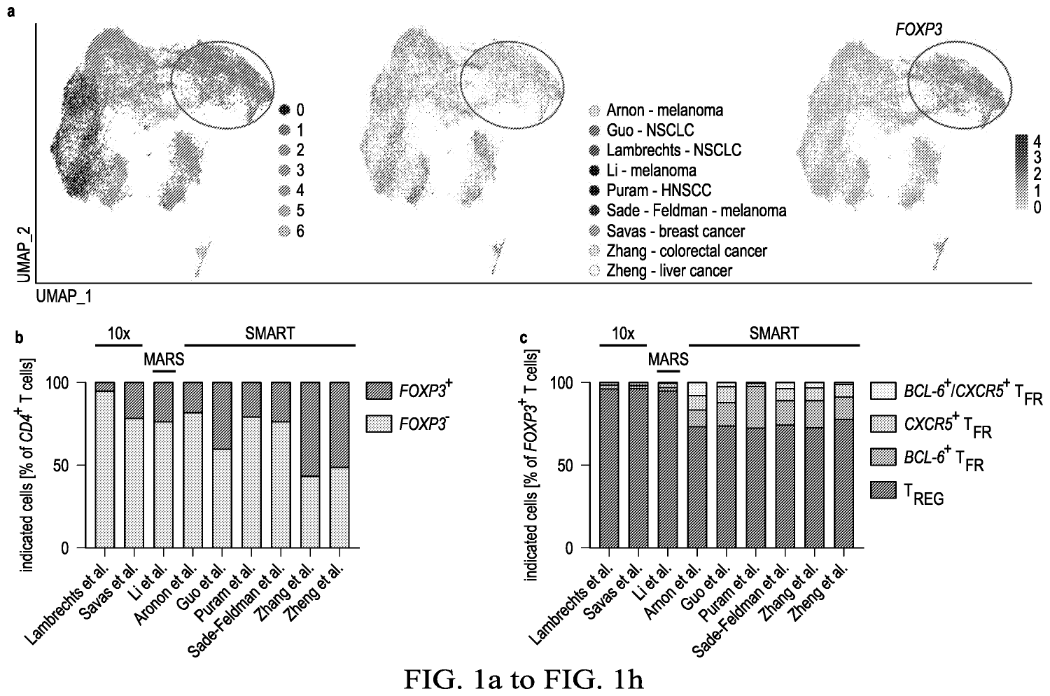

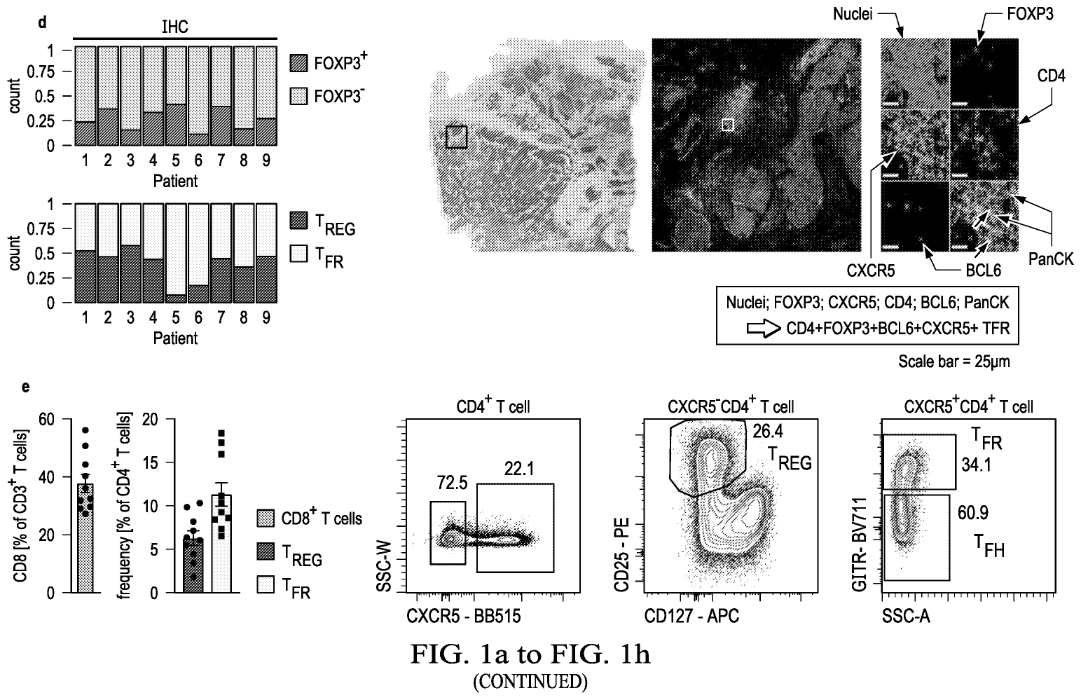

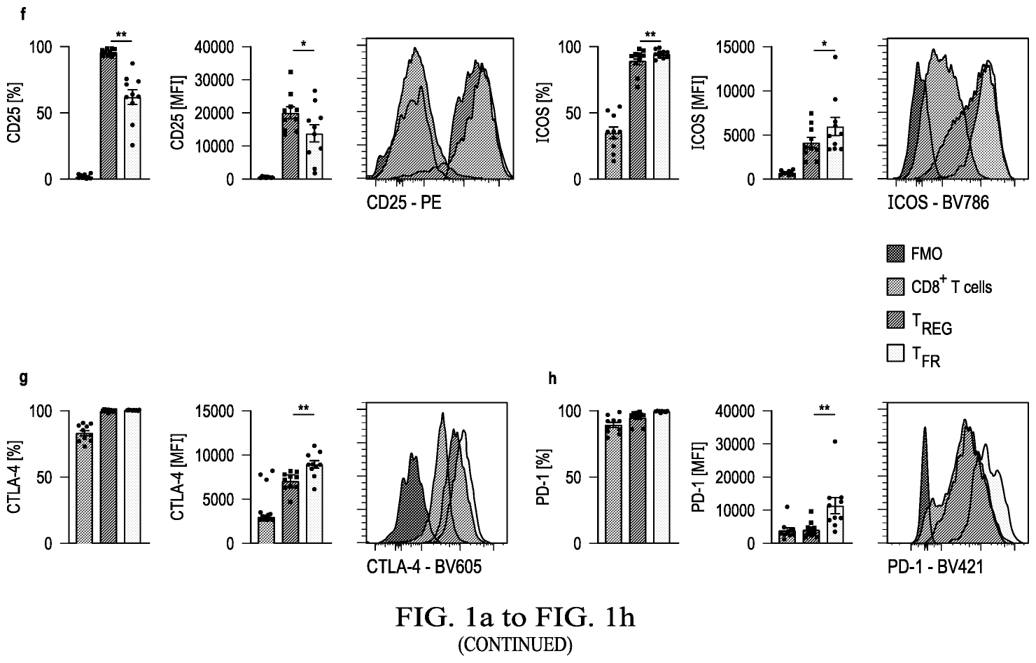

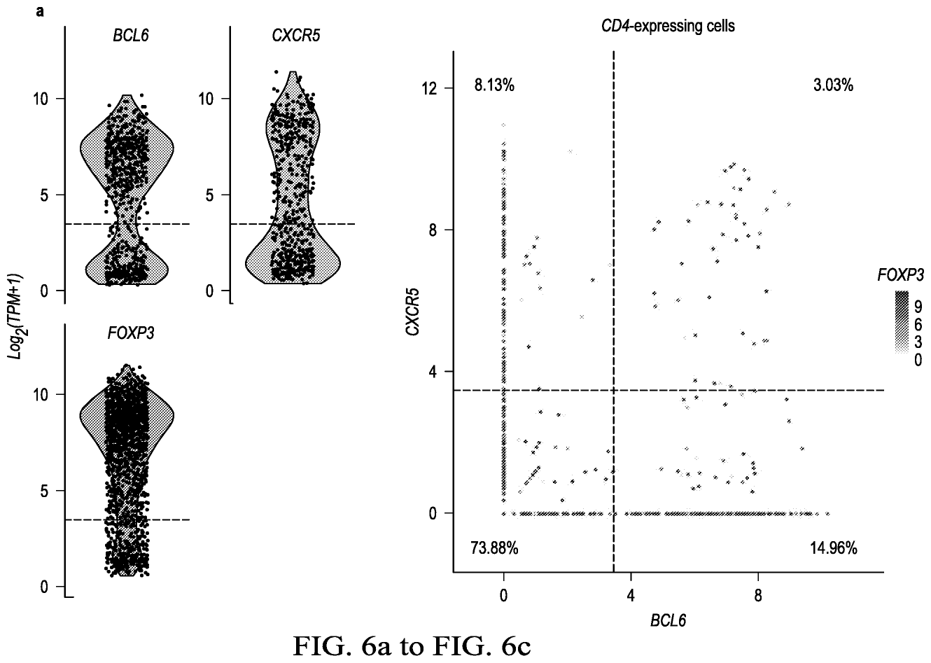

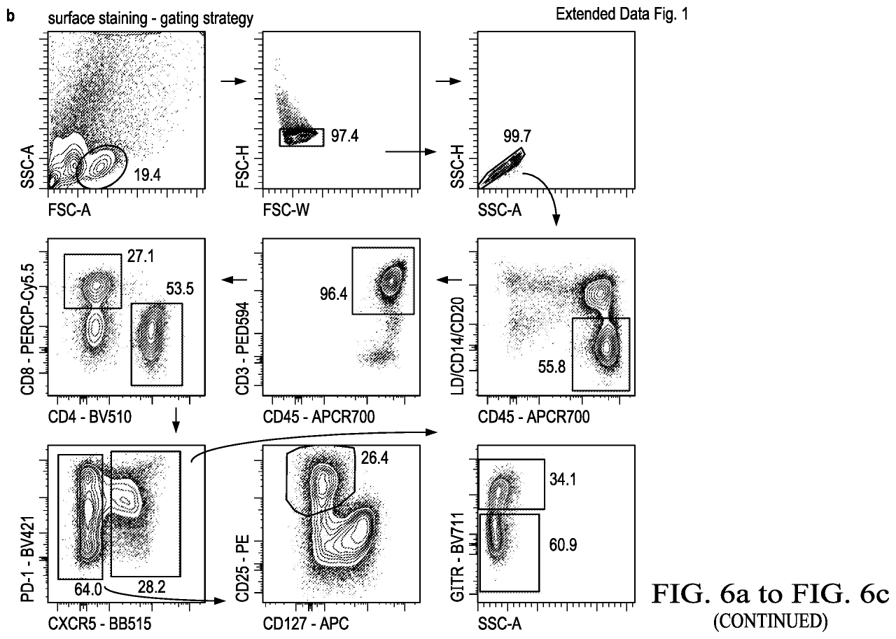

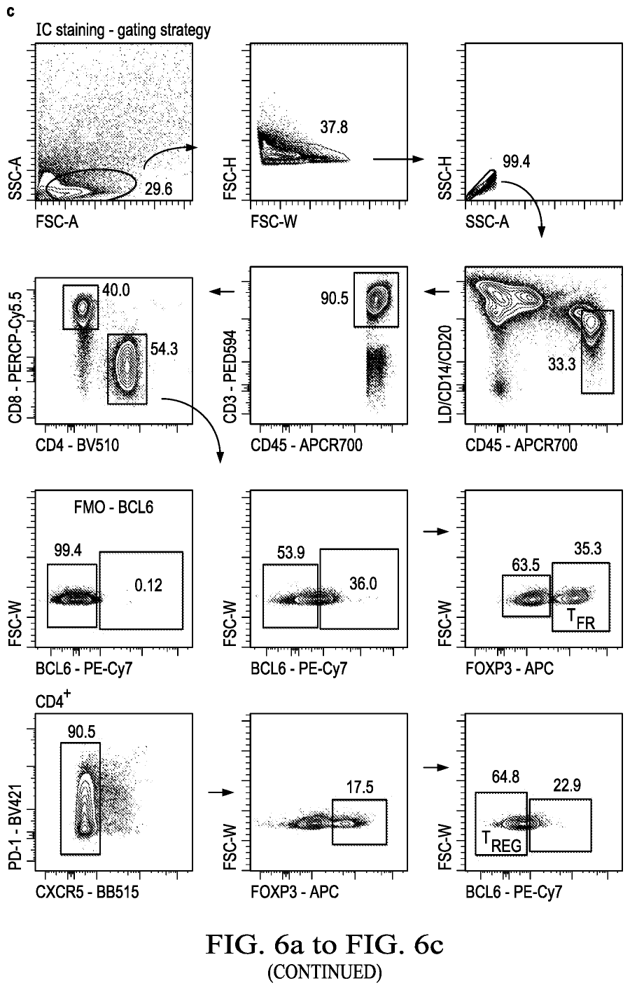

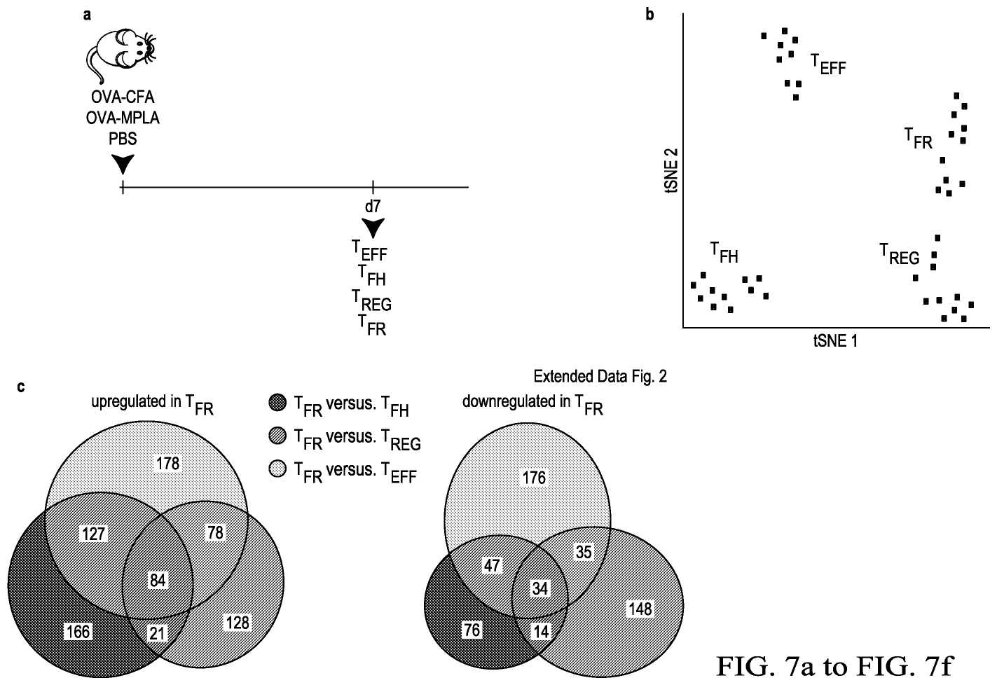

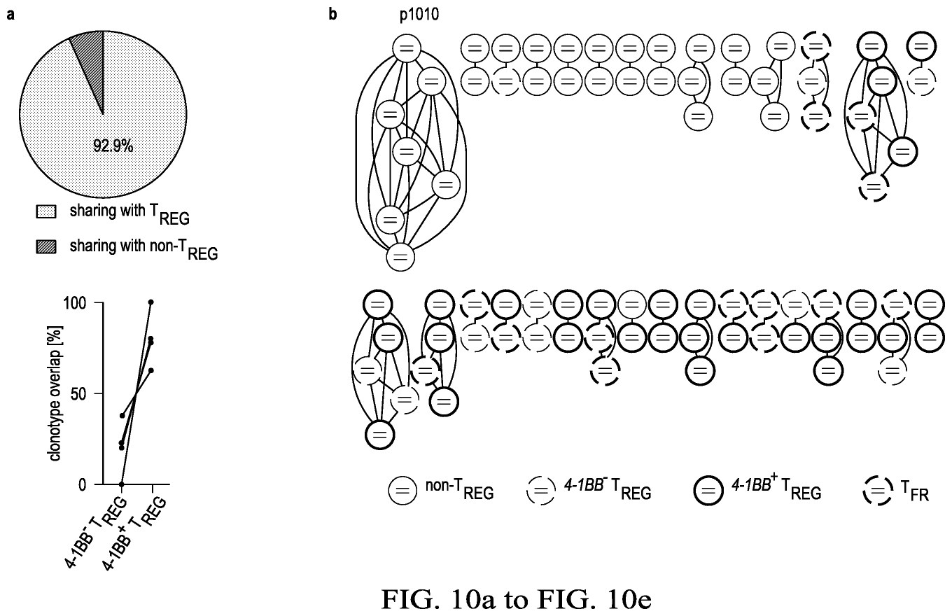

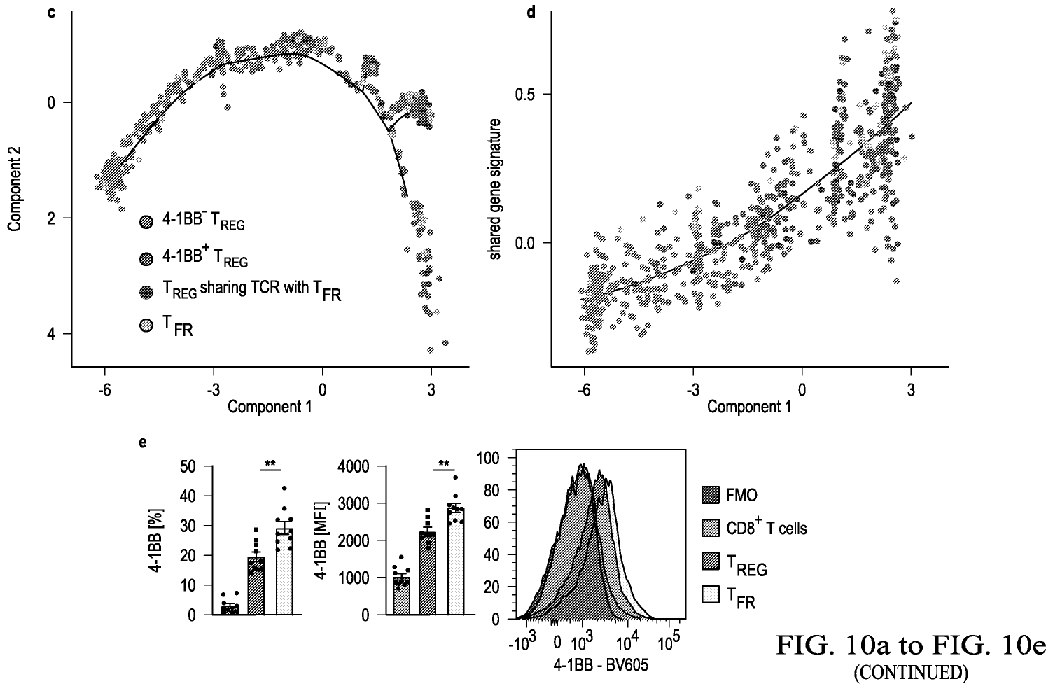

T_{FR} cells are present in multiple cancer types. The inventors integrated 9 published single-cell RNA-seq datasets and performed a meta-analysis of tumor-infiltrating CD4⁺ T cells (n=25,149) from patients with six different cancer types (Supplemental Data Table 1). As expected, FOXP3-expressing CD4⁺ T cells (i.e., T_{REG} cells) clustered distinctly and represented 5-55% of all tumor-infiltrating CD4⁺ T cells (FIG. 1a, 1b). The inventors found that a substantial proportion (5-30% in all tumor types) of FOXP3-expressing CD4⁺ T cells co-expressed BCL6 and/or CXCR5 (FIG. 1c and FIG. 6a), which encode for markers indicative of cells of a follicular lineage in humans and mice^{2,12}, and thus represent tumor-infiltrating T_{FR} cells, an important regulatory subset that has not been appreciated so far. The inventors confirmed the presence (˜10% of all tumor-infiltrating CD4⁺ T cells) and localization of T_{FR} cells in tumor samples from patients with treatment-naïve early-stage non-small cell lung cancer (NSCLC) by immunohistochemistry and multi-parameter flow cytometry (FIG. 1d-1h, FIG. 6b, 6c). T_{FR} cells, like T_{REG} cells, maintained surface expression of CD25 and ICOS (FIG. 10. To determine if currently available immunotherapies, like anti-CTLA4 and anti-PD1 therapies, also target tumor-infiltrating T_{FR} cells, the inventors assessed their expression of CTLA-4 and PD-1. Notably, T_{FR} cells expressed the highest levels of CTLA-4 and PD-1 compared to T_{REG} cells and CD8⁺ TILs (FIG. 1f,h), suggesting that anti-CTLA-4 can more efficiently target T_{FR} cells, and that anti-PD-1 therapies may inadvertently activate such suppressive T_{FR} cells.

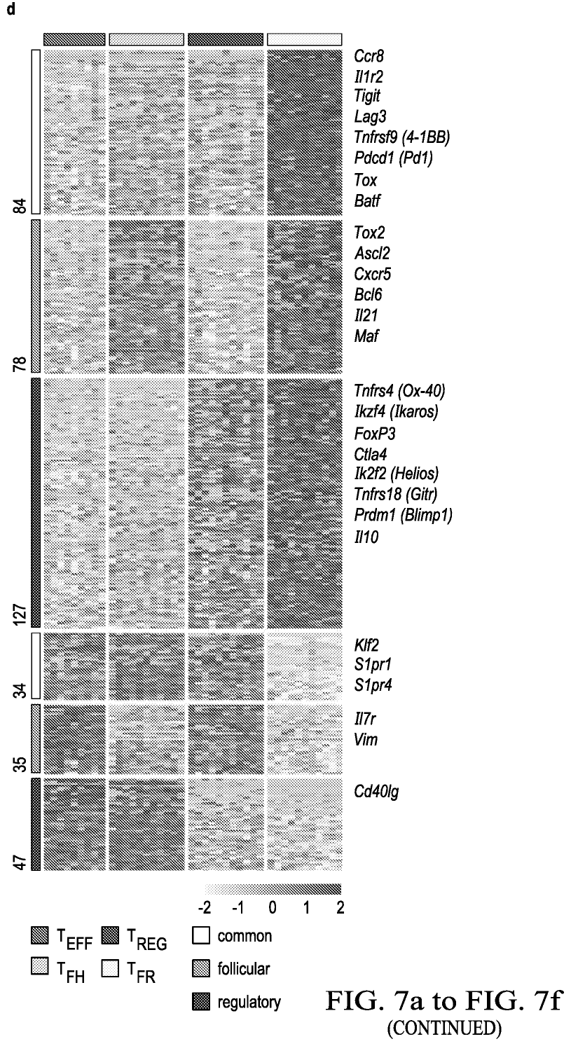

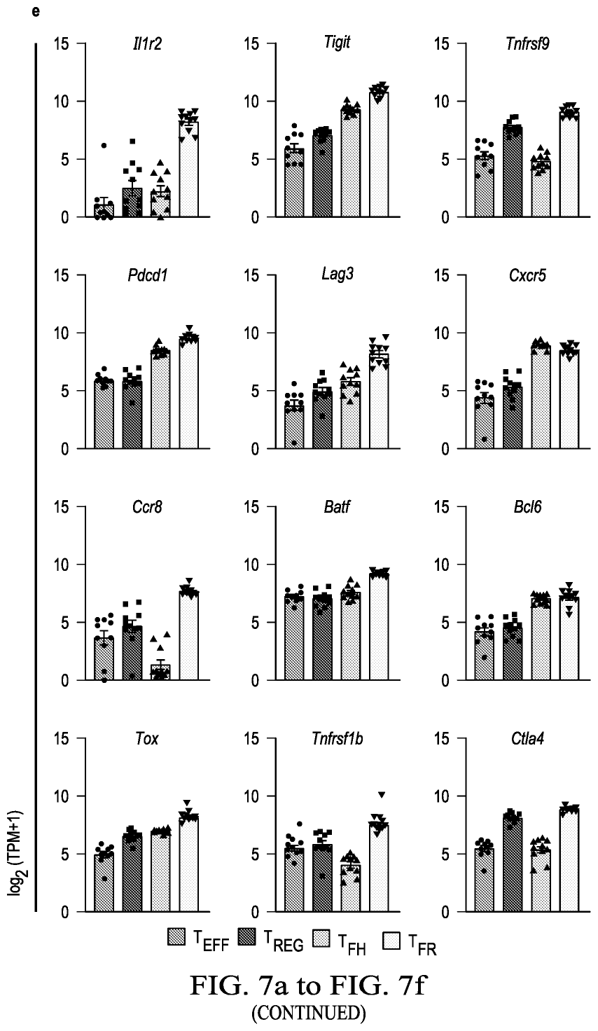

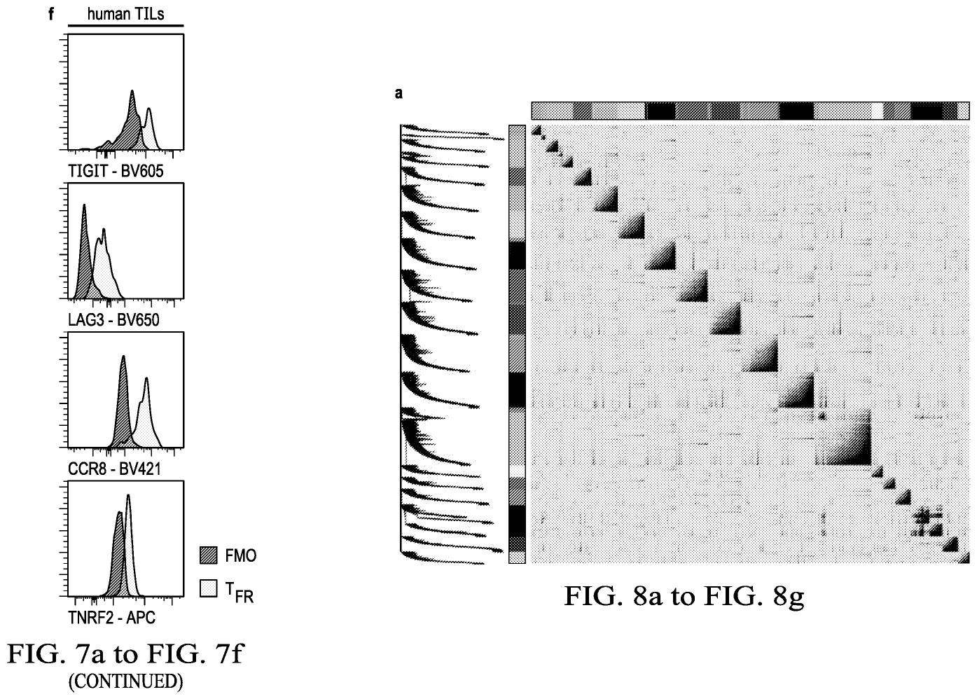

T_{FR} cells exhibit unique transcriptomic features. As few studies have thoroughly analyzed the transcriptomic features of T_{FR} cells, the inventors first utilized well-established immunization models in mice to gain mechanistic insights into T_{FR} cell function and to assess whether the features identified in human tumor-infiltrating T_{FR} cells are also applicable to murine T_{FR} cells. Immunization with ovalbumin and adjuvant (CFA or MPLA) induced robust T_{FR} responses (FIG. 7). Comparative analysis of their transcriptome with that of other T_{H} subsets showed increased expression of many transcripts specifically in T_{FR} cells (n=84), (FIG. 7b-7e), and notably the transcripts enriched in T_{REG} cells compared to both T_{FH} and T_{EFF} populations (n=127) were also highly expressed in T_{FR} cells (FIG. 7d, 7e). These include several transcripts (e.g., Tnfrsf1b¹³, Lag3¹⁴, Tigit¹⁵, Batf¹⁶, and Illr2^{1,17}) encoding for products associated with heightened suppressive capacity; Ccr8, which was associated with particularly poor clinical outcomes in cancer^{1,18}, and genes associated with CD8⁺ T cell dysfunction and survival¹⁹ (Pdcd1 and Tox) (FIG. 7d, 7e). The protein expression levels of some of these molecules, e.g., TNFR2 (encoded by TNFRSF1B), LAG3, TIGIT and CCR8, were confirmed in human tumor-infiltrating T_{FR} cells (FIG. 7f), suggesting suppressive capacity of T_{FR} cells and likely conservation of functional potential across species.

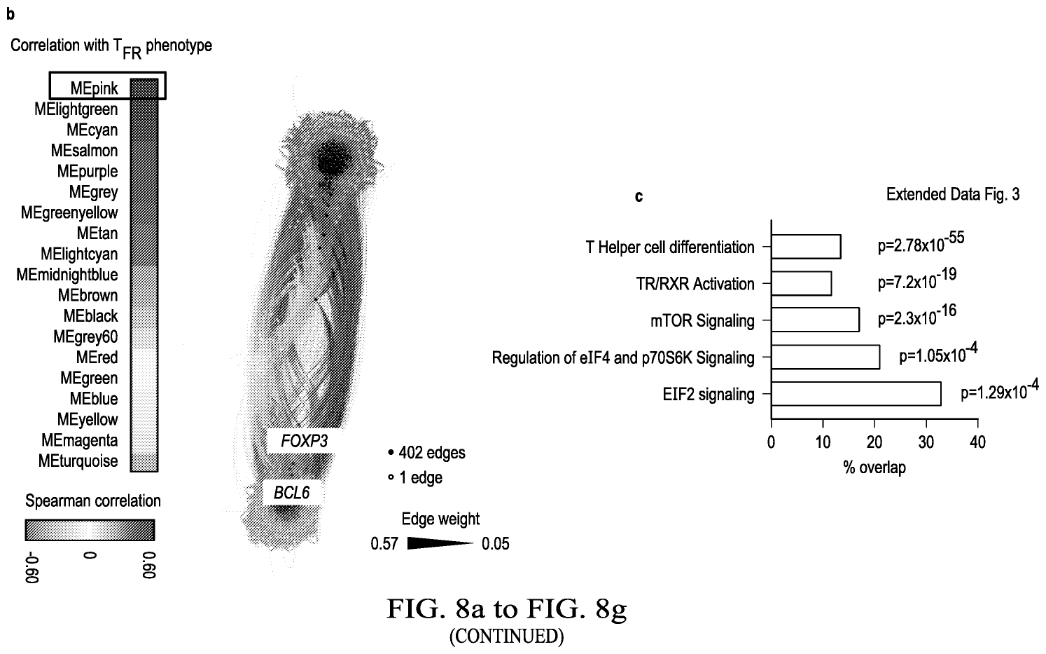

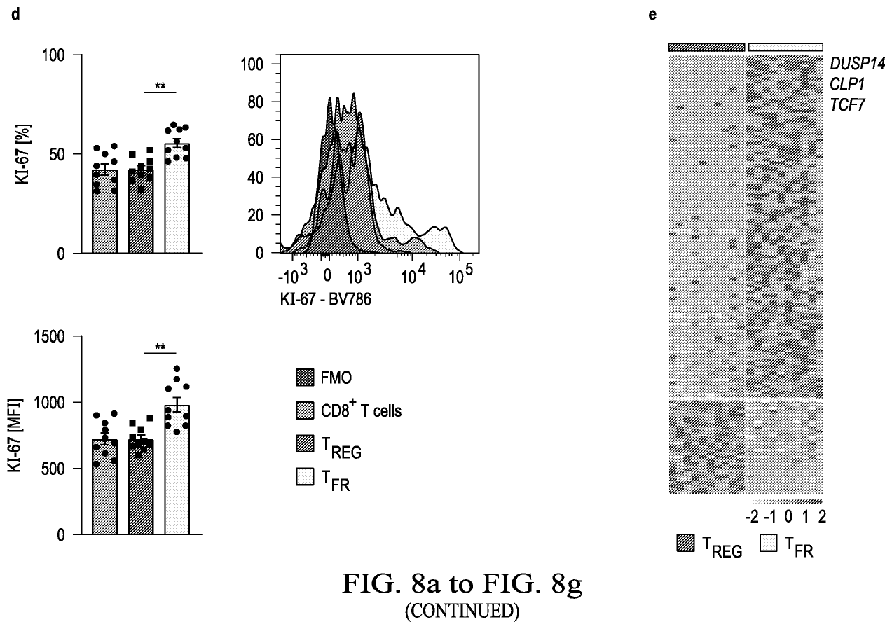

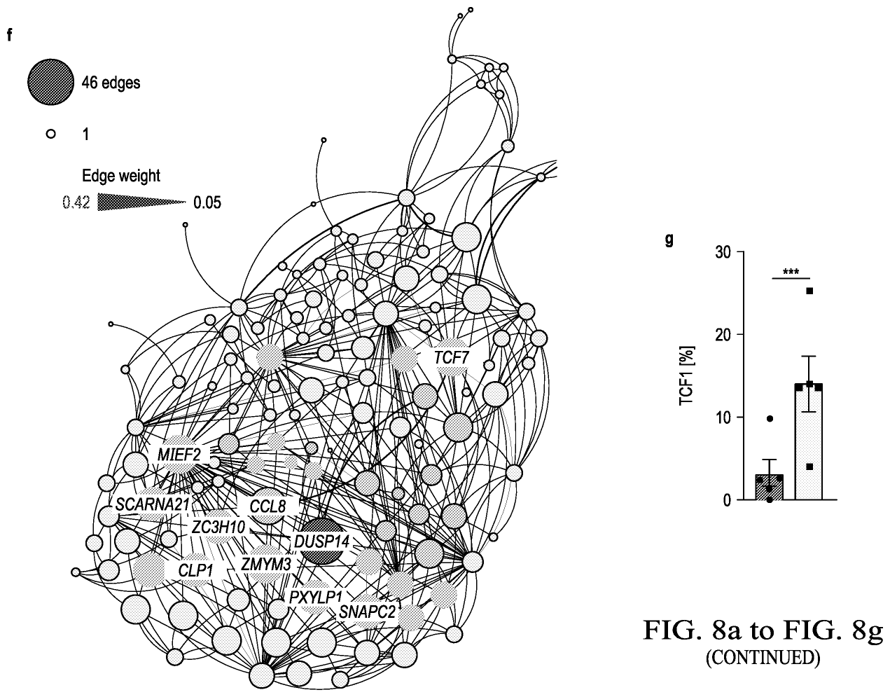

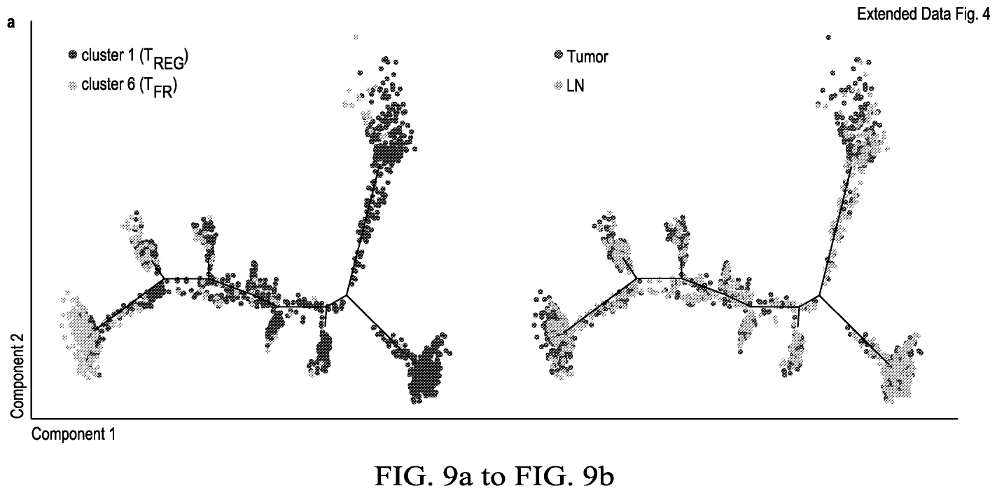

Next, the inventors performed bulk RNA-seq analyses of enriched populations of T_{REG} (CD4⁺CD25⁺CXCR5⁻) and T_{FR} cells (CD4⁺CXCR5⁺GITR⁺) (FIG. 6b) isolated from tumor samples of NSCLC patients. Weighted gene co-expression network analysis (WGCNA) (FIG. 8a and Supplemental Table 2) of bulk-sorted human T_{REG} (CD4⁺CD25⁺CXCR5⁻) and T_{FR} cells (CD4⁺CXCR5⁺GITR⁺) (FIG. 6b) identified a module (pink) that was positively correlated with the T_{FR} phenotype (FIG. 8b). Importantly, this module contained both BCL6 and FOXP3, demonstrating the linked expression of these genes, specifically in T_{FR} cells. Ingenuity Pathway Analysis (IPA) of the pink module (module positively correlated with T_{FR} phenotype) identified substantial enrichment of genes involved in cell cycle, transcriptional and translational activity and mTOR signaling, indicative of increased T_{FR} cell proliferation and activity (FIG. 8c). The inventors confirmed that T_{FR} cells indeed showed greater cell proliferation in the TME as evidenced by increased Ki-67 staining (FIG. 8d). Differential gene expression analysis of enriched populations of T_{FR} cells and T_{REG} cells identified over 100 transcripts that were expressed at higher levels in T_{FR} cells (FIG. 8e). Co-expression analysis of these differentially expressed transcripts revealed a number of highly correlated novel genes (e.g. DUSP14, CLP1), which may play a role in T_{FR} cell function. Moreover, the inventors identified TCF7 (encoding TCF1) as a highly connected hub gene in this transcriptomic network (FIG. 80 and confirmed that the proportion of TCF-1-expressing cells was higher in T_{FR} cells compared to T_{REG} cells (FIG. 8g). Interestingly, TCF1-expressing CD8⁺ CTLs have recently been recognized for their ability for self-renewal, stem-like properties^{20,21} and their pivotal role in mediating anti-cancer immune attack induced by anti-PD-1 immunotherapy^{22,23}, suggesting that TCF-1 expression might confer similar features on T_{FR} cells.

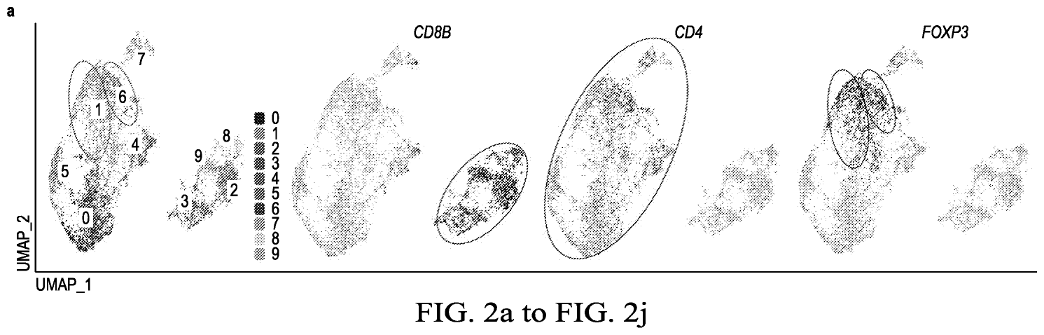

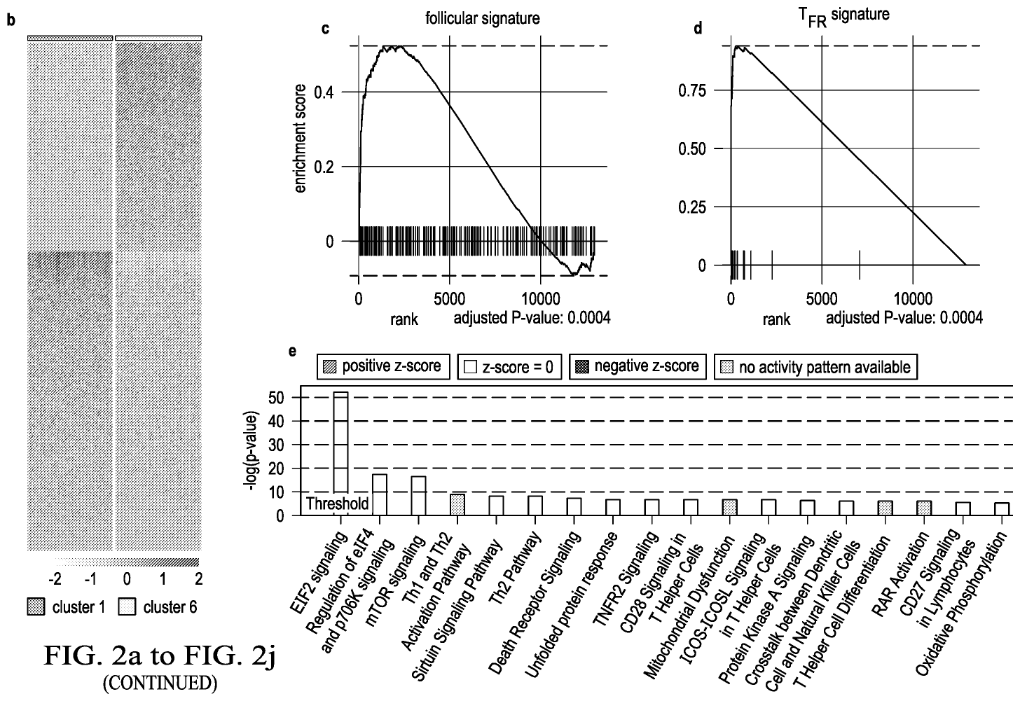

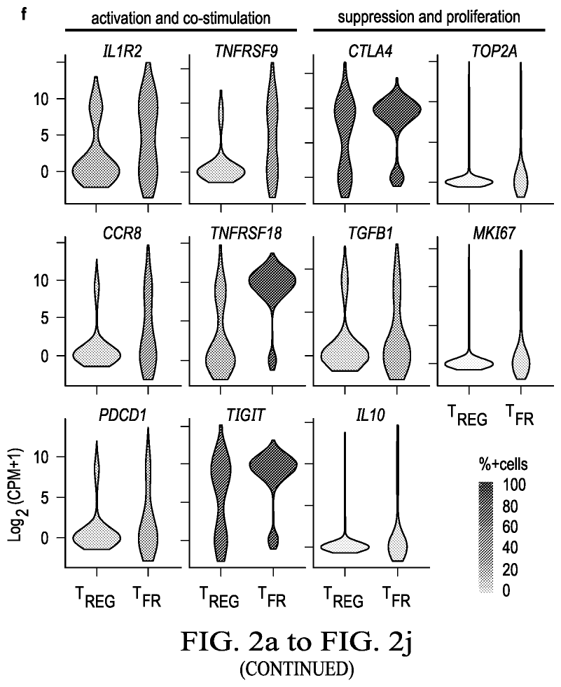

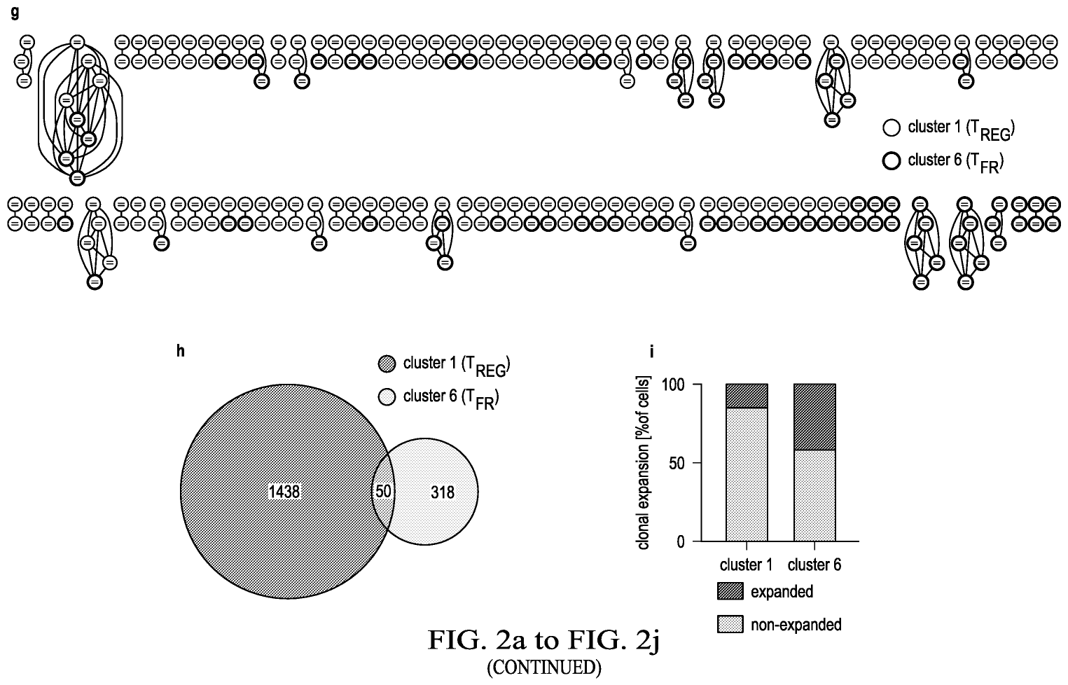

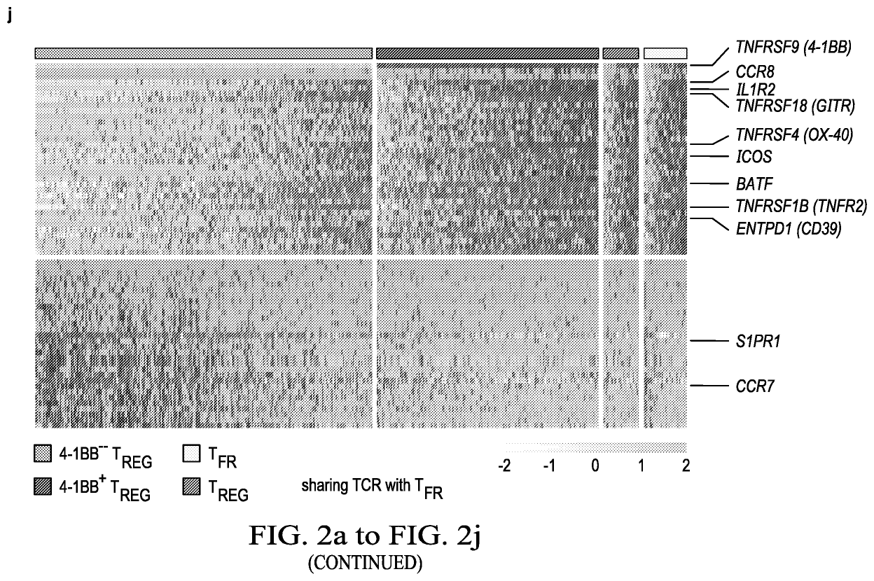

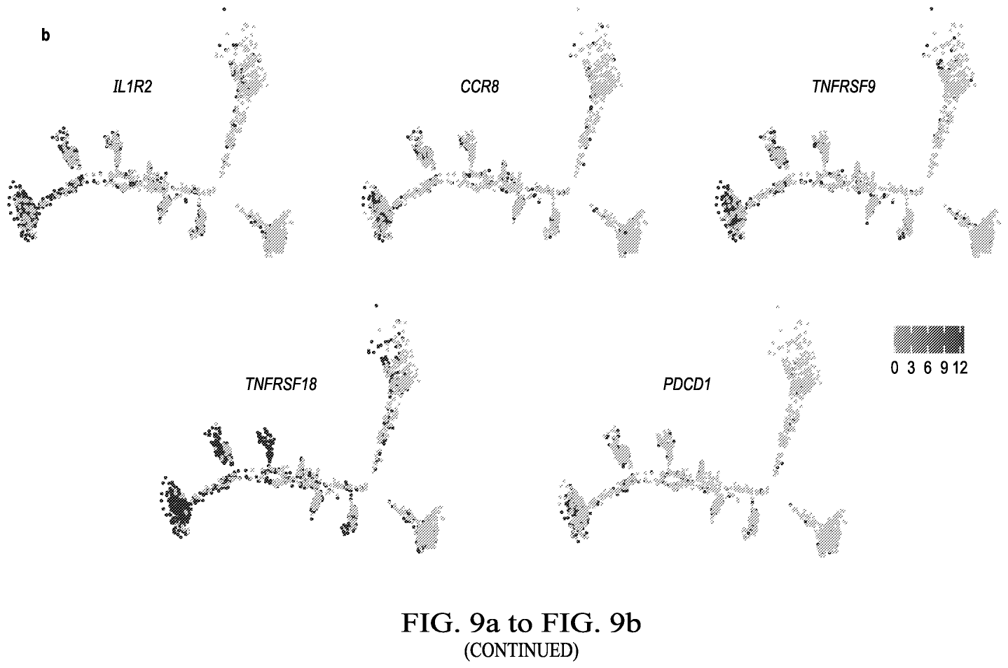

Intratumoral T_{REG} and T_{FR} cells share clonotypes but differ in their molecular profile. Recent data demonstrate that tumor-infiltrating T_{REG} cells potently recognize tumor (neo)antigens and, upon antigen-encounter, undergo clonal expansion²⁴. Given that antigen-specific activation of T_{REG} cells in the context of viral infection has been implicated in promoting their differentiation into T_{FR} cells via Tcf1-mediated induction of Bcl6²⁵, the inventors hypothesized that tumor-associated antigen (TAA) recognition may also trigger T_{REG} to T_{FR} conversion within the TME. To assess this, the inventors performed combined single-cell RNA-seq and TCR-seq of sorted CD4⁺ (T_{FH}, T_{REG} and T_{FR}) and CD8⁺ TILs from primary tumor tissue and tumor-infiltrated lymph nodes of two HNSCC patients (n=8,722 cells). Unsupervised clustering revealed two distinct CD4⁺ T cell clusters (1 and 6) that were enriched for FOXP3 expression (FIG. 2a), and which exhibited distinct transcriptomic signatures (FIG. 2b and Table 3). Gene set enrichment analysis showed that cells in cluster 6 (yellow) were significantly enriched for follicular (FIG. 2c) and T_{FR} cell signatures (FIG. 2d), thus characterizing T_{FR} cells, while cells in cluster 1 (green) depict T_{REG} cells. Pathway analysis of the differentially expressed genes (FIG. 2b) between T_{FR} and T_{REG} cells showed enrichment for transcripts linked to metabolism, cell activation and co-stimulation (FIG. 2e and Table 2). Moreover, T_{FR} cells expressed higher levels of transcripts liked to T_{FR} function and suppressive capacity (e.g., CTLA4, IL10, TGFB1, TNFRSF9, or IL1R2), and cell cycle genes (TOP2A, MKI67) (FIG. 2f). Accordingly, although T_{REG} and T_{FR} cells shared clonotypes (FIG. 2g, 2h), T_{FR} cells were more clonally expanded than T_{REG} cells (FIG. 2i). Importantly, TCR sharing and trajectory analysis of cells in the FOXP3-enriched clusters indicate intratumoral conversion of T_{REG} to T_{FR} cells (FIG. 2g, 2h and FIG. 9a, 9b). To further substantiate this notion, the inventors re-analyzed one of the largest single-cell RNA-seq datasets¹⁷ of tumor-infiltrating CD4⁺ T cells, showing that the majority of clonally-expanded T_{FR} clonotypes (˜93%) were shared with T_{REG} cells (FIG. 10a, upper panel). Furthermore, T_{REG} cells that shared clonotypes with T_{FR} cells predominantly expressed 4-1BB (TNFRSF9) transcripts (FIG. 10a, lower panel and FIG. 10b), implying recent TCR activation²⁶, and indicative of potential intratumoral conversion of TAA-activated T_{REG} to T_{FR} cells. Trajectory analysis implies that 4-1BB⁺ T_{REG} cells (TAA-experienced) and T_{REG} cells sharing TCRs with clonally-expanded T_{FR} cells (purple) depict transitional states during differentiation of T_{REG} cells into T_{FR} cells (FIG. 10c). Importantly, transcripts linked to cell activation, co-stimulation and suppressive function (FIG. 2j) were expressed at higher levels in 4-1BB⁺ T_{REG} cells (red), TCR sharing T_{REG} cells (purple) and clonally-expanded T_{FR} cells (yellow) compared to 4-1BB⁻ T_{REG} cells (green), a gene signature that was highly associated with Monocle component 1 (FIG. 2j and FIG. 10d). When compared to 4-1BB⁻ T_{REG} cells, T_{FR} cells and T_{REG} cells on their trajectory to differentiate into T_{FR} cells also showed significant downregulation of CCR7 and S1PR1, genes that encode receptors required for tissue egress and thus support tissue residency²⁷ (FIG. 2j).

TABLE 3
List of differentially expressed genes when comparing
4-1BB− TREG cells with three populations: 4-
1BB+ TREG, clonally-expanded TREG cells sharing
their TCRs with TFR and clonally-expanded TFR cells

| Gene ID | Mean Log2 fold change | Mean Adjusted P-value |
|---|---|---|
| TNFRSF9 | 7.28 | 1.43E−09 |
| CCR8 | 3.00 | 4.76E−03 |
| IL1R2 | 3.83 | 4.56E−06 |
| TNFRSF18 | 2.54 | 2.94E−02 |
| TNFRSF4 | 2.37 | 7.03E−03 |
| LAYN | 2.41 | 1.24E−02 |
| CST7 | 1.95 | 5.50E−02 |
| LAIR2 | 2.41 | 5.76E−03 |
| CXCR6 | 1.94 | 1.15E−01 |
| ICOS | 2.12 | 3.20E−03 |
| IL21R-AS1 | 2.24 | 6.68E−02 |
| CD27 | 1.83 | 2.65E−02 |
| BATF | 1.94 | 2.33E−03 |
| DUSP4 | 2.22 | 1.32E−03 |
| SDC4 | 2.17 | 5.26E−02 |
| APOBEC3C | 1.55 | 1.07E−01 |
| CD177 | 2.68 | 1.56E−02 |
| COX5A | 1.66 | 1.03E−01 |
| GCNT1 | 2.12 | 1.98E−02 |
| ZBTB32 | 2.78 | 7.98E−04 |
| ENTPD1 | 1.78 | 2.39E−02 |
| MIR4632 | 2.13 | 9.55E−04 |
| TYMP | 1.89 | 4.20E−02 |
| TNFRSF1B | 1.86 | 2.06E−03 |
| SAT1 | 1.68 | 1.82E−02 |
| VDR | 1.74 | 3.24E−02 |
| NAMPT | 1.51 | 5.58E−02 |
| ASB2 | 2.01 | 5.80E−03 |
| BEX3 | 2.07 | 4.56E−02 |
| ID3 | 2.19 | 9.34E−02 |
| MAP2K3 | 1.76 | 5.92E−02 |
| CCL22 | 3.41 | 1.28E−02 |
| GAPDH | 1.11 | 3.25E−02 |
| AHSP | 3.09 | 2.34E−03 |

These observations are consistent with a model in which the TME is initially infiltrated by a large and highly diverse pool of bystander (i.e., not TAA-specific) T_{REG} cells, and a smaller pool of TAA-specific T_{REG} clones, which are poised for differentiation into tissue resident T_{FR} cells. This implies that T_{FR} cells comprise a larger proportion of tumor-reactive clones than T_{REG} cells, which is substantiated herein by the finding that T_{FR} cells expressed significantly higher levels of 4-1BB than T_{REG} cells (FIG. 10e). These highly suppressive TAA-reactive T_{FR} cell clones could eventually outcompete the bystander T_{REG} cells, as evidenced by their higher proliferative capacity, and potentially shield tumor cells expressing these antigens against immune-attack by inhibiting priming of CD4+ 28,29 and CD8⁺ CTLs³⁰.

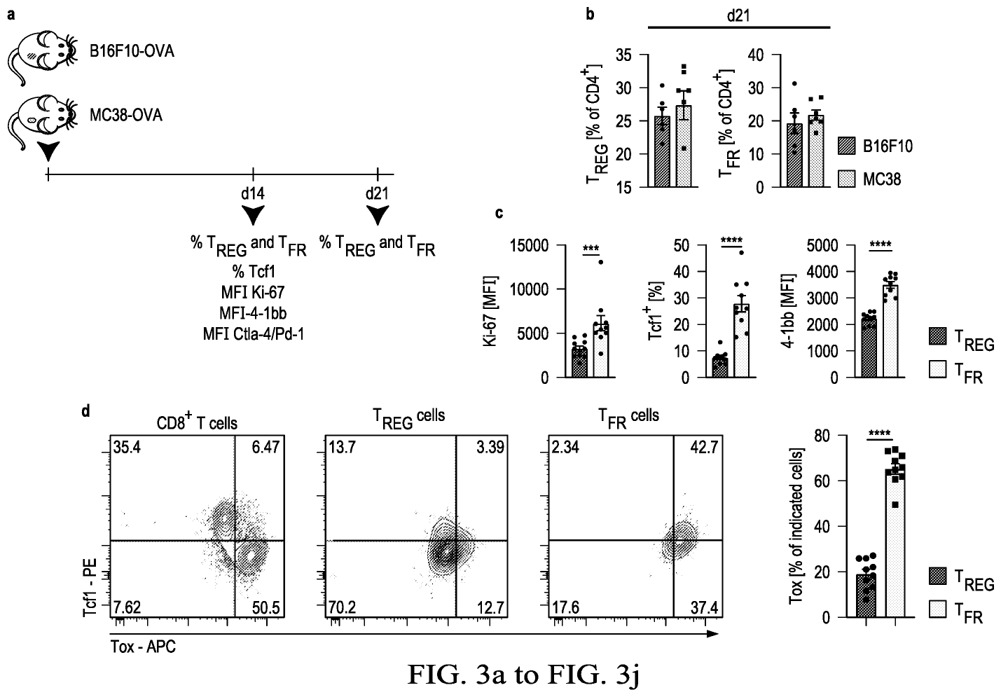

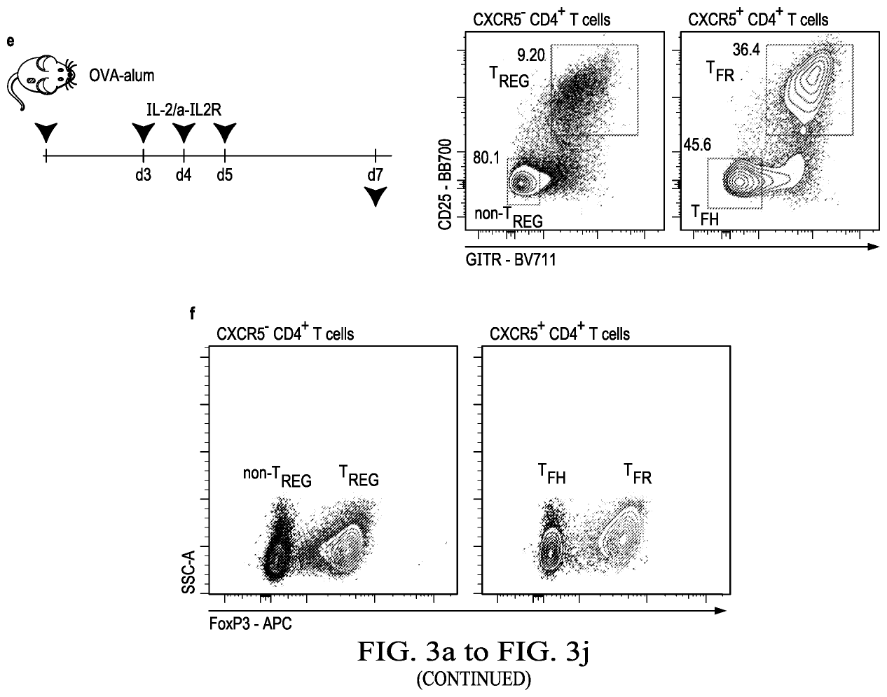

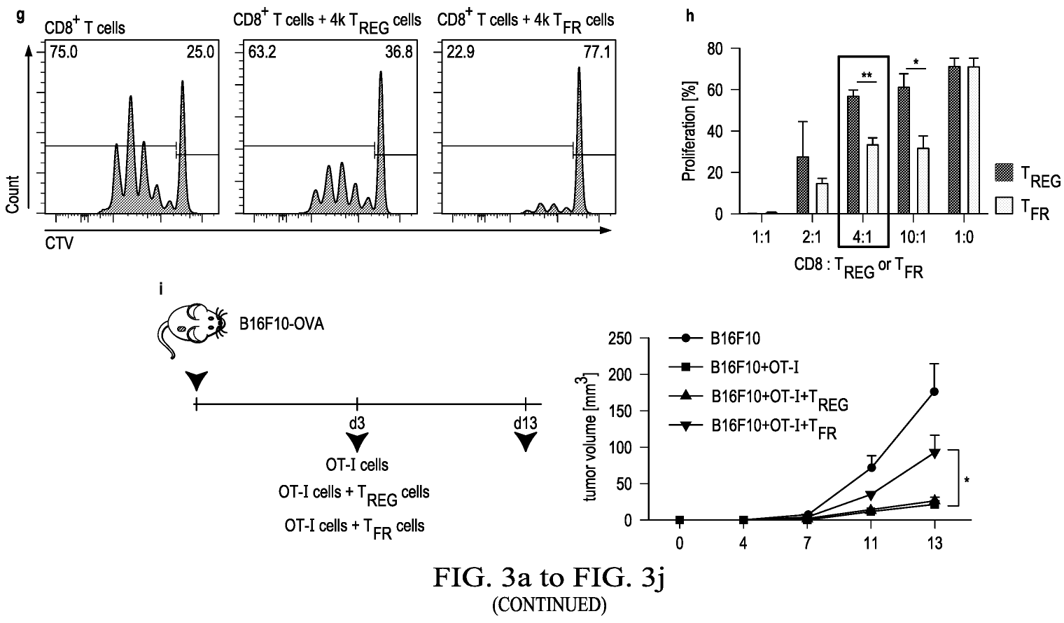

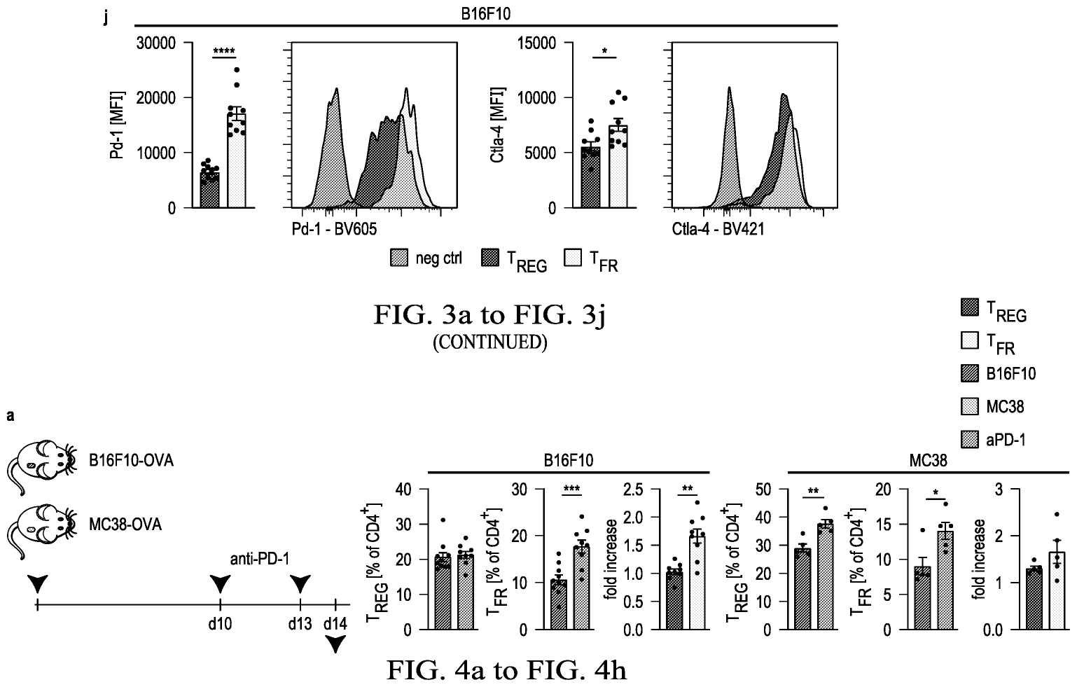

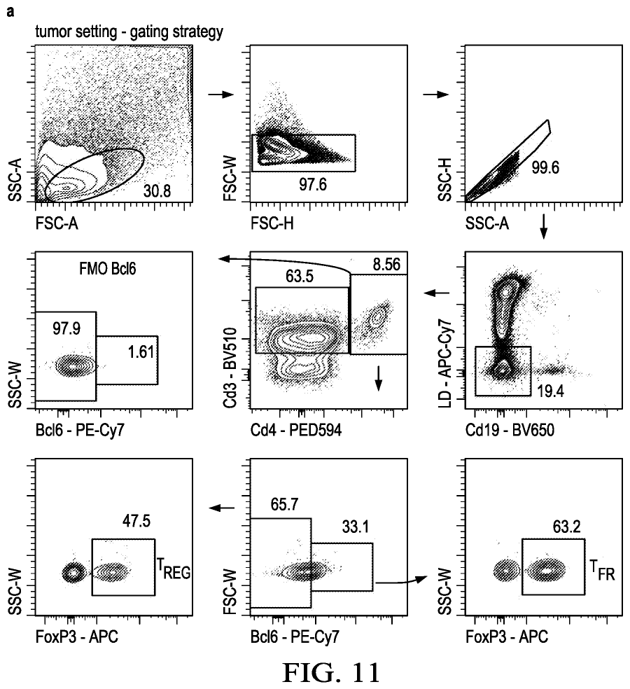

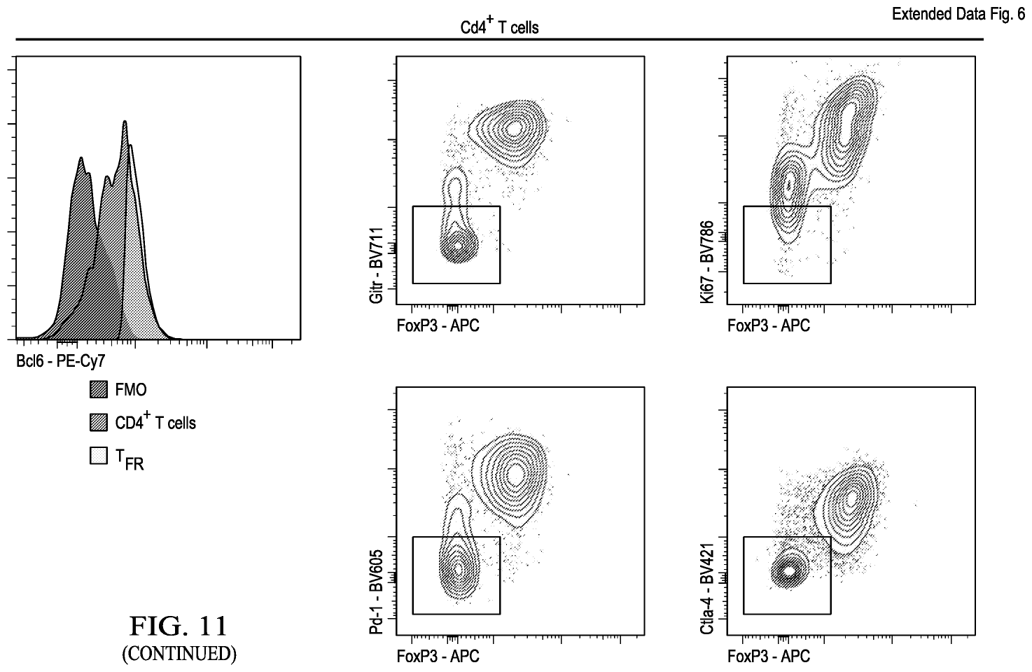

T_{FR} cells exhibit superior suppressive capacity. Next, the inventors assessed frequency, activity and functional responsiveness of T_{FR} cells in murine tumor models. T_{FR} cells (CD3⁺CD4⁺Bcl6⁺Foxp3⁺) were present in tumor samples from two syngeneic tumor model systems (B16F10 melanoma and MC38 colorectal tumor cell lines) (FIG. 3a, 3b and FIG. 11a), but importantly lacked expression of Cxcr5. Notably, recent studies demonstrated that ablation of Cxcr5 expression in FoxP3⁺ T cells did not abrogate the development of Bcl6⁺ T_{FR} cells, which still entered the germinal center reaction⁷. Thus, even in the absence of Cxcr5, Bcl6 expression in FoxP3⁺ cells still delineates T_{FR} cells.

Similar to human T_{FR} cells, murine T_{FR} cells exhibited increased proliferative potential, as evidenced by Ki-67 expression levels, and increased expression of Tcf1 and 4-1bb compared to T_{REG} cells (FIG. 3c). Interestingly, T_{FR} cells also expressed significantly higher levels of transcription factor Tox (FIG. 3d).

To experimentally validate that T_{FR} cells are more suppressive than T_{REG} cells, the inventors performed functional assays in vitro and in vivo. Strikingly, it was found that T_{FR} cells suppressed CD8⁺ T cell proliferation significantly more efficiently than T_{REG} cells (FIG. 3e-h). Based on these results, the inventors chose to transfer OT-I T cells, either alone or with T_{REG} or T_{FR} cells in a 4:1 ratio (FIG. 3h), into B16F10-OVA tumor-bearing RAG1^{-/-} recipient mice. While the effect of adoptively transferred T_{REG} cells was negligible, T_{FR} cells substantially inhibited OT-I T cell-mediated tumor rejection (FIG. 3i), demonstrating that T_{FR} cells exhibit superior suppressive potential when compared to T_{REG} cells. Murine tumor-infiltrating T_{FR} cells also showed higher expression of Ctla-4 and Pd-1 when compared to T_{REG} cells (FIG. 3j), implying that such murine tumor models would be appropriate to test the hypothesis that anti-PD1 therapy increases the numbers and/or function of highly suppressive T_{FR} cells and induces a profoundly immunosuppressive tumor milieu. Since PD-1^{-/-} mice exhibit increased levels of T_{FR} cells in secondary lymphoid organs⁶, the inventors reasoned that PD-1 signaling is likely to restrain expansion of T_{FR} cells.

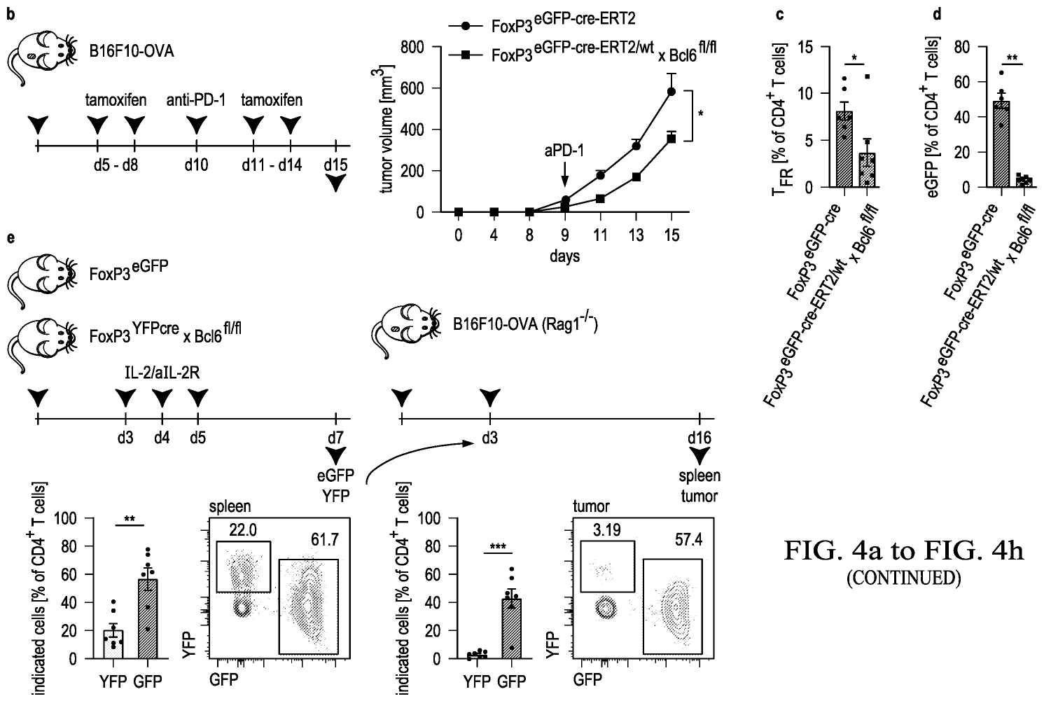

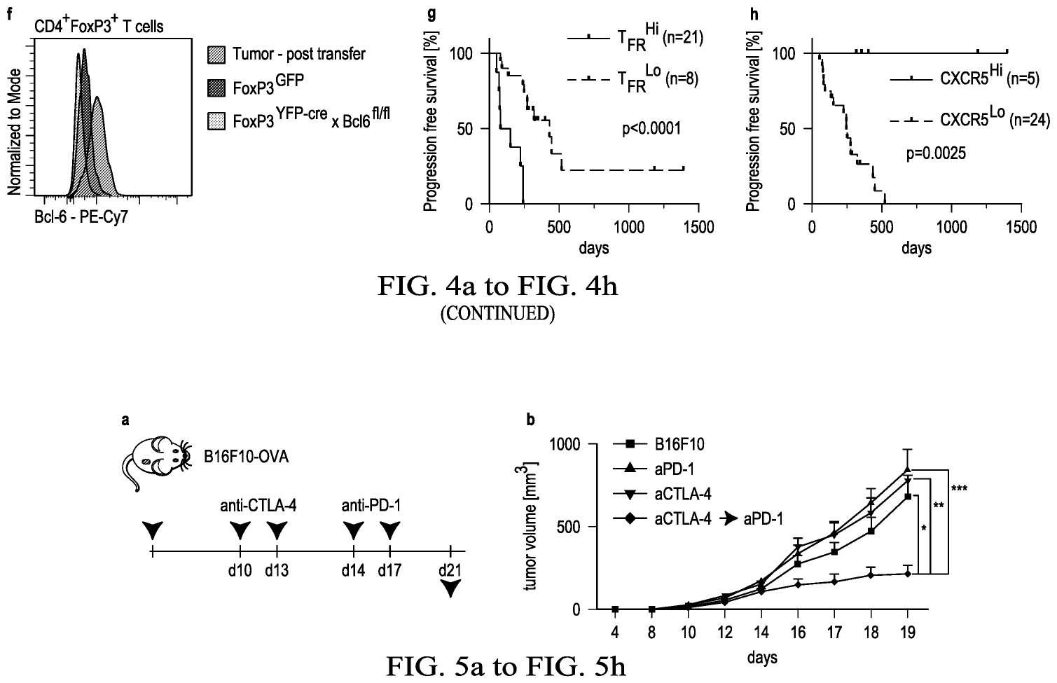

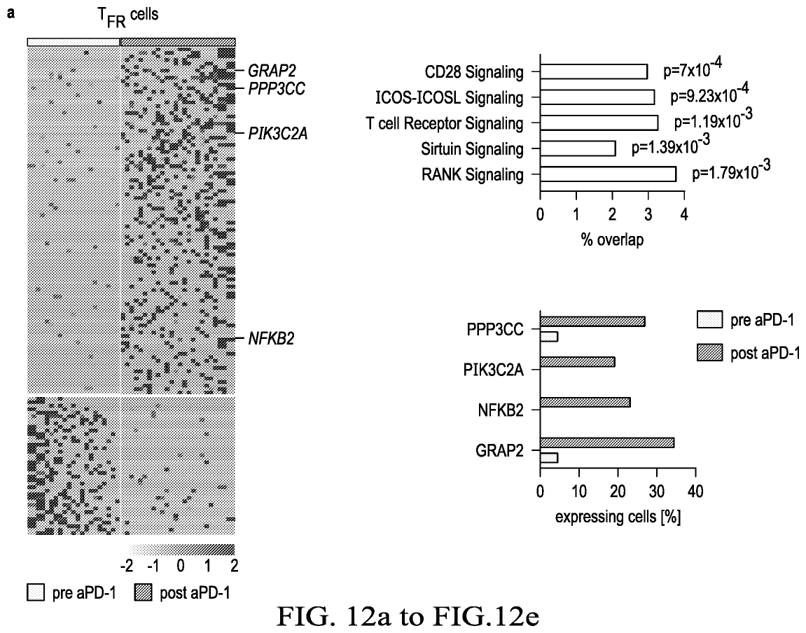

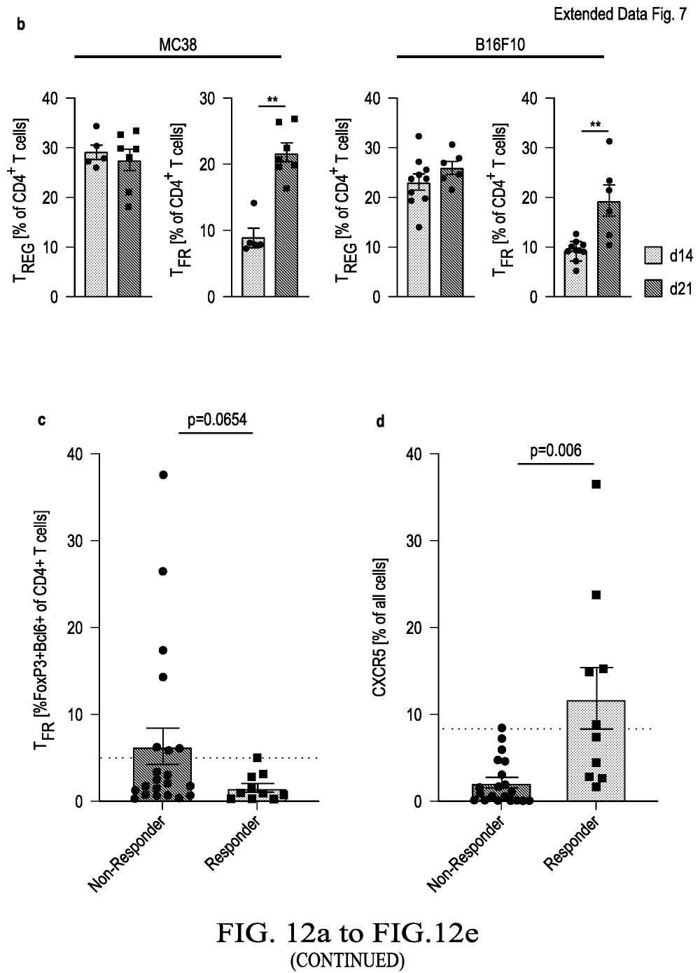

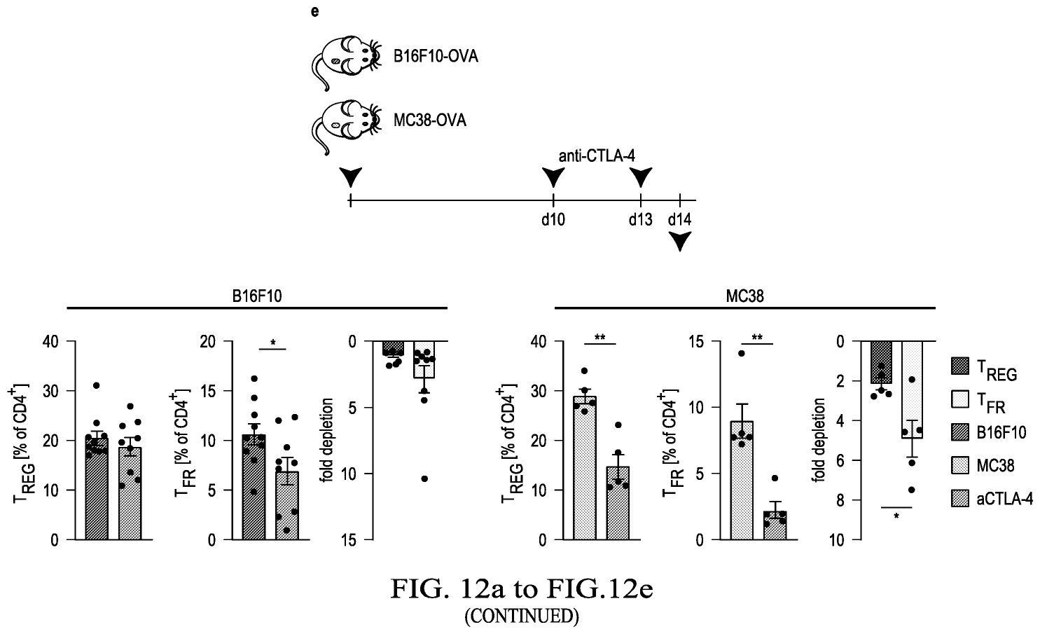

T_{FR} cells are responsive to anti-PD-1 therapy. Anti-PD-1 monotherapy resulted in a significant increase in the frequency of T_{FR} cells in both MC38 and B16F10 tumor models (FIG. 4a), suggesting that tumor-infiltrating T_{REG} (and T_{FR} cells) are highly responsive to blockade of PD-1 signaling, potentially reducing their activation threshold and thus facilitating increased proliferation and differentiation into T_{FR} cells. By re-analyzing published single-cell RNA-seq data from patients receiving anti-PD-1 therapy, the inventors found that tumor-infiltrating T_{FR} cells from post-treatment samples compared to pre-treatment samples were enriched for transcripts linked to T cell activation and co-stimulation (FIG. 12a). Together, these data suggest that engagement of suppressive T_{FR} cells by anti-PD1 therapy is likely to diminish its anti-tumor efficacy.

To uncouple the effects of T_{REG} and T_{FR} cells on anti-tumor immunity and anti-PD-1 treatment efficacy, the inventors utilized a genetic knockdown system, in which T_{FR} cells can be selectively depleted. Tamoxifen-induced depletion of T_{FR} cells in female heterozygous FoxP3^{eGFP-cre-ERT2cre/wt}×Bcl6^{fl/fl} mice³³ prior to initiation of anti-PD-1 therapy, significantly decreased tumor growth, demonstrating that T_{FR} cells curtail anti-PD-1 treatment efficacy (FIG. 4b). In this system, half of the FoxP3⁺ cells should express the FoxP3^{eGFP-cre-ERT2} allele (eGFP⁺ and Bcl6-deficient) due to random X chromosome inactivation. Surprisingly however, while the inventors found a decrease in T_{FR} cells (FIG. 4c), the inventors observed a near total loss of Bcl6-deficient eGFP⁺ T_{REG} cells in the TME (FIG. 4d). Crucially, these data imply that even a partial T_{FR} cell depletion decreases tumor growth, and that Bcl6 expression in FoxP3⁺ cells is likely to be required for their intratumoral persistence. These findings raise several distinct conclusions explaining for the accumulation of eGFP⁻FoxP3⁺ cells; (i) Bcl6-deficiency affects trafficking of T_{REG} cells into tumor tissue. (ii) lack of Bcl6 expression precludes adoption of a tissue-residency program, and (iii) Bcl6-deficient T_{REG} cells are being outcompeted by their Bcl6-sufficient counterparts. By way of explanation, and in no way a limitation of the present invention, the latter hypothesis is supported by the inventors' prior observations of increased proliferative potential, as well as higher expression of Tox and Tcf1 by Bcl6⁺ T_{FR} cells compared to Bcl6⁻ T_{REG} cells (FIG. 3c, 3d). To experimentally validate that T_{FR} cells have greater in vivo persistence in tumor tissue, the inventors performed a competition assay, where the inventors co-transferred FoxP3⁺eGFP⁺ cells from FoxP3^{eGFP}mice (capable of producing Bcl6) and FoxP3⁺YFP⁺ cells from FoxP3^{YFP-cre}×Bcl6^{fl/fl} mice (incapable of producing Bcl6) in a 1:1 ratio. Strikingly, FoxP3⁺YFP⁺ cells failed to accumulate in the spleen and TME (FIG. 4e) of B16F10-OVA tumor-bearing Rag1^{-/-} mice, demonstrating that FoxP3⁺Bcl6⁺ cells (T_{FR} cells) are better suited to survive in the TME. Importantly, the transferred tumor-infiltrating FoxP3⁺ cells expressed significantly higher levels of Bcl-6 when compared to pre-transfer levels, indicative of intratumoral T_{REG} to T_{FR} conversion (FIG. 4f), corroborating the previous hereinabove (FIG. 2g-2i and FIG. 10a-10c). Based on these findings, the inventors performed a time course experiment which confirmed that the proportion of T_{FR} cells, but not T_{REG} cells, increased with tumor progression (FIG. 12), likely reflective of ongoing T_{REG} to T_{FR} conversion and higher proliferative potential of T_{FR} cells.

In an independent cohort of patients with melanoma (n=29), who received anti-PD1 treatment, the inventors observed poor survival outcomes in patients with a higher proportion of CD4⁺ T cells co-expressing FOXP3 and BCL6 (BCL6⁺ T_{FR} cells) in tumor (FIG. 4g), and also noticed a trend towards a higher frequency of BCL6⁺ T_{FR} cells in non-responders compared to responders to anti-PD-1 treatment (FIG. 12c). A lower frequency of CXCR5⁺ cells (FIG. 12d), a surrogate marker for the abundance of tertiary lymphoid structures, was also associated with poor survival outcomes (FIG. 4h), consistent with recently published studies³⁴⁻³⁶. Given that T_{FR} cells have been shown to mitigate germinal center responses in secondary lymphoid organs, it is tempting to assume that T_{FR} cells might not only impede anti-tumor immunity by inhibiting CD8⁺ TILs (FIG. 3d-h), but also by regulating tertiary lymphoid structures in tumor tissues, which should be investigated in future studies.

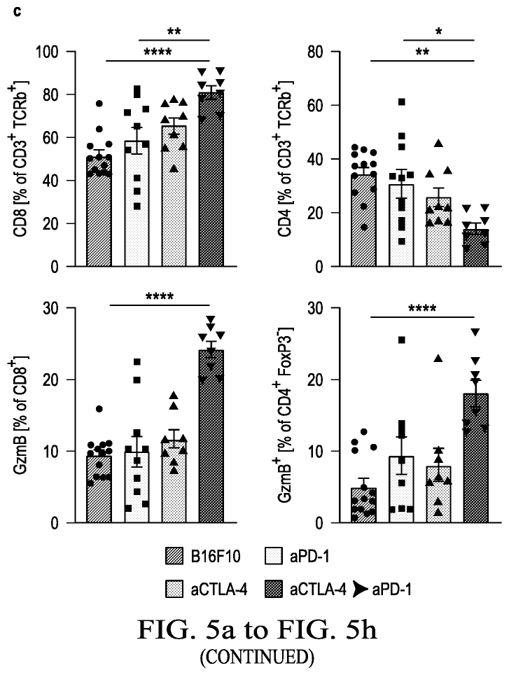

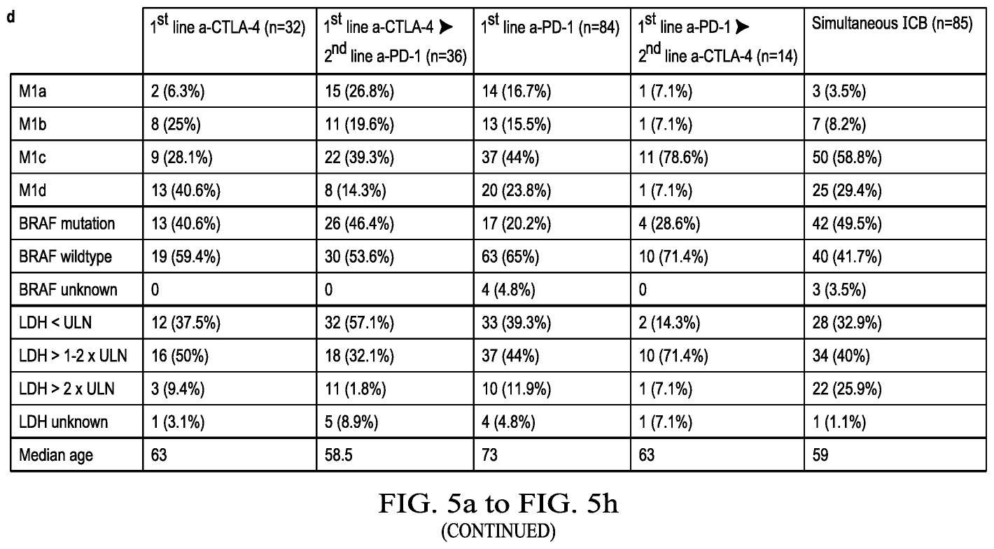

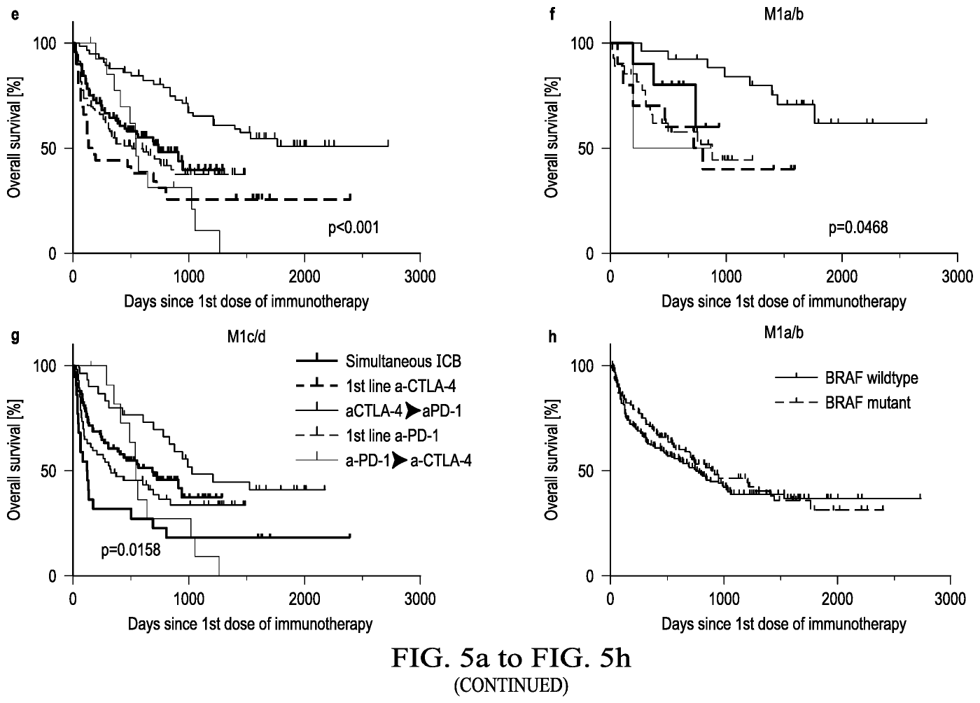

Sequential ICB is beneficial in melanoma patients. To overcome the suppressive milieu induced by anti-PD-1-mediated increase in T_{FR} cells, the inventors reasoned that it may be necessary to deplete T_{FR} cells in the tumor prior to initiating anti-PD1 therapy. Anti-CTLA-4 treatment is believed to deplete intratumoral T_{REG} cells via antibody-dependent cellular cytotoxicity³⁷. Given that tumor-infiltrating T_{FR} cells expressed higher levels of CTLA-4 than T_{REG} cells in both human and mouse (FIGS. 1g and 3j), the inventors hypothesized that T_{FR} cells should be more efficiently depleted. Indeed, anti-CTLA-4 monotherapy resulted in greater depletion of T_{FR} cells compared to T_{REG} cells (FIG. 12e). These data also indicate that immunotherapy drugs elicit immediate effects on target cell populations and rapidly re-shape the cellular composition within the TME. Based on these results, the inventors reasoned that sequential immune checkpoint blockade (ICB) treatment (FIG. 5a), where T_{FR} cells are initially depleted by anti-CTLA-4, might prove beneficial, as subsequent anti-PD-1 therapy would not activate suppressive cellular targets (T_{FR} cells) but would instead engage CD8⁺ TILs to enhance anti-tumor immune responses. The inventors chose to test this hypothesis in the B16F10-OVA melanoma model as it is known to be refractory to anti-PD-1 therapy³⁸. As expected, monotherapy with either anti-CTLA-4 or anti-PD-1 antibodies did not impact tumor growth, whereas depletion of T_{FR} cells with anti-CTLA4 followed by anti-PD1 therapy led to a significantly reduced tumor volume (FIG. 5b). It was found that anti-PD-1 therapy increasingly acts on, and hence elevates the frequency of CD8⁺ TILs after T_{FR} cells have been depleted by anti-CTLA-4 treatment (FIG. 5c), and also led to an increase in the frequency of GzmB⁺ CD8⁺ and CD4⁺ CTLs (FIG. 5c).

To test the clinical significance of sequential ICB treatment, the inventors retrospectively assessed the survival outcomes of patients with inoperable melanoma (n=271), who were, based on their treatment regimens, stratified into 5 groups: 1^{st} line anti-CTLA-4, 1^{st} line anti-PD-1, simultaneous combination therapy, sequential therapy with anti-CTLA-4 followed by anti-PD-1 at progression and vice versa. Sequential treatment with anti-CTLA-4 followed by anti-PD-1 was associated with better long-term overall survival (OS) outcomes when compared to the 4 other groups (p<0.001) (FIG. 5d, 5e). It has to be noted though that patients receiving simultaneous ICB therapy exhibited a more advanced disease prior to treatment initiation (higher proportion with AJCC 8 stage M1c and M1d (n=75) than patients on 1^{st} line anti-PD-1 (n=70) or 1^{st} line anti-CTLA-4 (n=52), (FIG. 5f), likely contributing to their poor OS outcomes. However, the advantageous effect of anti-CTLA-4 followed by anti-PD-1 therapy was preserved in patients with M1a/b and M1c/d, respectively (FIG. 5f, 5g), indicating that this treatment regimen is clinically beneficial. Differences in BRAF status did not affect ICB treatment outcomes (FIG. 5h). The outcome data for patients receiving 1^{st} line anti-CTLA-4 appear to be superior to those in a recently published study³⁹, but are however not directly comparable, as the proportions of patients going on to receive 2^{nd} line anti-PD-1 treatment was significantly lower in that trial (43% vs 63%). Crucially, in mouse models (FIG. 5a), when compared to monotherapy with anti-PD1, sequential treatment with anti-CTLA-4 (likely to deplete T_{FR} cells in the tumor) followed by anti-PD-1 was associated with significantly better survival outcomes (p=0.0003) (FIG. 5e).

In summary, these results demonstrate in both human and murine tumors, the existence of suppressive and highly proliferative T_{FR} cells that, among tumor-infiltrating lymphocytes, expressed the highest levels of CTLA-4 and PD-1. The findings in the murine tumor model demonstrate that intratumoral T_{FR} cells are responsive to ICB and that by increasing the abundance of T_{FR} cells, anti-PD-1 therapy can not only facilitate, but also dampen anti-tumor immune attack. The inventors provide critical insights into how anti-CTLA-4 and anti-PD-1 therapies mediate their function, and highlight the clinical benefit of sequential dosing to render tumors responsive to anti-PD1 therapy. The well-described clinical scenario in which some tumors hyper-progress following anti-PD1 therapy^{40,41} is finally explained due to the effects of treatment on a highly suppressive immune cell compartment (T_{FR} cells), especially in patients with an initially high level of tumor-specific T_{REG} (T_{FR} precursor) cells. Conversely, exacerbated immune-related adverse events observed upon combination therapy, might be caused by anti-CTLA-4-mediated depletion of suppressive T_{FR} cells and subsequent uninhibited anti-PD-1-mediated activation of effector CD4⁺ and CD8⁺ T cells, hypotheses which can be addressed in future studies. Finally, these results provide for the first time a unique composition of stimulatory versus suppressive T cells in the TME of each patient, as well as their differentiation status (i.e., PD-1 expression levels and frequency of T_{FR} cells within CD4⁺ T cells), as important immunological determinants driving anti-PD-1 treatment efficacy.

Human tumor samples. The study was approved by the Southampton and South West Hampshire Research Ethics Board, and written informed consent was obtained from all subjects. Newly diagnosed, untreated patients with NSCLC, were prospectively recruited once referred. Freshly resected tumor tissue was obtained from lung cancer patients following surgical resection and after histological confirmation. The patient cohort for the survival analysis was collected by retrospective evaluation of a centralized prescribing system (Aria, Varian Medical Systems Inc, Crawley, UK). All patients started on immunotherapy at a single institution (Southampton University Hospitals NHS Foundation Trust) with immunotherapy for melanoma between July 2014 to October 2018 were included. Patients were divided into cohorts according to first type immunotherapy treatment approved in the United Kingdom (aPD-1, either nivolumab or pembrolizumab, N=98), aCTLA-4 antibody (ipilimumab (88) or joint administration of nivolumab plus ipilimumab on up to four occasions (N=85), followed by maintenance nivolumab where appropriate. Dosing was according to standard of care at the time (3 mg/kg ipilimumab×4, 2 mg/kg of pembrolizumab 3 weekly, later 200 mg flat dosing, 3 mg/kg nivolumab, then 480 mg flat dosing, and in combination 3 mg/kg ipilimumab+1 mg/kg nivolumab, four doses, followed by 3 mg/kg nivolumab). All patients were included who had at least one dose of immunotherapy. Clinical data were obtained from an electronic hospital record for age, gender, BRAF status, LDH, M stage, performance status. For clinical outcome overall survival was collected to death or censored at last clinical review. Data were anonymized by the treating clinician (I.K. and C.H.O.) once the data had been collated and verified. Prism 8 (Graph Pad Software, San Diego, USA) was used for ANOVA and to plot Kaplan Meier Survival Graphs and estimate treatment differences using a Log-rank (Mantel-Cox) test on survival curves. SPSS v26 (IBM Corp, New York, USA) was used to evaluate imbalances between treatment groups via Chi Square testing followed by Cox Regression analysis. Fur multiple testing a Bonferroni error correction was applied.

Mice. C57BL/6J, Bcl6^{fl/fl}, OT-I and RAG1^{-/-} mice were obtained from Jackson labs. FoxP3^{eGFP-cre-ERT2} and FoxP3^{YFP-cre} mice were a kind gift from Klaus Ley (LJI) and FoxP3^{eGFP} mice were a kind gift from Amnon Altman (LJI). All mice were between 6-12 weeks old at the beginning of experiments. All animal work was approved by the relevant La Jolla institute for Immunology Animal Ethics Committee.

Tumor cell lines. B16F10-OVA cells were a gift from the laboratory of Prof. Linden (LJI) and MC38-OVA cells were a gift from the lab Prof. Fuchs (UPenn) and approved for use by Prof. Smyth (Peter MacCallum cancer centre). B16F10-OVA cells form distinct melanoma tumors and are thus true melanoma cells. MC38-OVA cells were generated at the Peter MacCallum cancer centre and therefore authenticated by the developer. Cell lines tested negative for mycoplasma infection and were subsequently treated with Plasmocin to prevent contamination.

Tumor models. Tumor cell lines were tested negatively for mycoplasma infection and Plasmocin (InvivoGen) was used as a routine addition to culture media to prevent mycoplasma contamination. Mice were inoculated with 1-1.5×10⁵ B16F10-OVA cells or 2×10⁶ MC38-OVA cells subcutaneously into the right flank. Mice were injected intraperitoneally at indicated time points with either 200 μg anti-PD-1 (29F1.A12, InvivoPlus anti-mouse PD-1, Bioxcell), anti-CTLA-4 (9H10, InvivoPlus anti-mouse CTLA-4, Bioxcell) or respective isotype controls (anti-CTLA-4 isotype ctrl, InVivoPlus polyclonal Syrian hamster IgG, Bioxcell) (anti-PD-1 isotype control, InVivoPlus rat IgG2a isotype control, anti-trinitrophenol, Bioxcell). Tumor size was monitored every other day, and tumor harvested at indicated time points for analysis of tumor-infiltrating lymphocytes. Tumor volume was calculated as ½×D×d², where D is the major axis and d is the minor axis, as described previously⁴³.

Suppression and Competition Assay. Mice were immunized i.p. with Ovalbumin in alum (100 ug in 100 ul sterile PBS mixed with 100 ul 2% alum). At day 3-5 after immunization, mice were immunized i.p. with an IL-2/anti-IL-2Receptor complex (1 ug IL-2, 5 ug anti-IL-2Receptor Ab, mixed for 30 min at 37 degrees Celsius) to achieve polyclonal expansion of T_{REG} cells in vivo, as described previously⁴⁴. Lymphocytes (CD4⁺ and CD8⁺ T cells) were isolated from spleen by mechanical dispersion through a 70-μm cell strainer (Miltenyi) to generate single-cell suspensions. CD4⁺ and CD8⁺ T cells were purified (Stemcell) according to manufacturer's instructions.

In Vitro—CD8⁺ T cells were labelled with CellTrace Violet (CTV) (Thermofisher) and 40,000 cells were added to 96 well cell culture plated, pre-coated with anti-CD3, in 200 ul complete RPMI media. Purified CD4⁺ T cells were stained and different numbers of viable (Fixable Viability dye) T_{REG} cells (CD4⁺CXCR5⁻CD25⁺GITR⁺) or T_{FR} cells (CD4⁺CXCR5⁺CD25⁺GITR⁺) were sorted into the cell culture plate containing the CTV-labeled CD8⁺ T cells. CD8⁺ T cell proliferation (CTV dilution) was determined 3 days later.

In Vivo—OT-I CD8⁺ T cells were purified (Stemcell), CD4⁺ T cells were purified, stained and T_{REG} and T_{FR} cells were sorted as described above. Cells were counted and 2×10⁵ OT-I T cells, 2×10⁵ OT-I T cells+5×10⁴ T_{REG} cells (4:1 ratio) or 2×10⁵ OT-I T cells+5×10⁴ T_{FR} cells (4:1 ratio) were adoptively transferred into B16F10-OVA tumor-bearing Rag1^{-/-} recipient mice 3 days after tumor inoculation.

Competition assay—OT-I CD8⁺ T cells were purified (Stemcell), FoxP3⁺ T cells were purified from FoxP3^{YFP-cre}×Bcl6^{fl/fl} mice (YFP⁺) and FoxP3^{eGFP} mice (GFP⁺) and 4×10⁵ cells (2×10⁵ OT-I T cells, 1×10⁵ GFP⁺ T_{REG} cells and 1×10⁵YFP⁺ T_{REG} cells) were adoptively transferred into B16F10-OVA tumor-bearing Rag1^{-/-} recipient mice 3 days after tumor inoculation.

Flow cytometry. Human T cells were isolated from cryopreserved tumor tissue using a combination of enzymatic and mechanical dissociation, as previously described⁴⁵. Cells were prepared in staining buffer (PBS with 2% FBS and 2 mM EDTA), FcR blocked (clone 2.4G2, BD Biosciences) and stained with indicated primary antibodies for 30 minutes at 4° C.; secondary stains were done for selected markers. Samples were then sorted or fixed and intracellularly stained using a FoxP3 transcription factor kit according to manufacturer's instructions (eBioscience). Cell viability was determined using fixable viability dye (ThermoFisher).

Murine samples—Lymphocytes were isolated from spleen by mechanical dispersion through a 70-μm cell strainer (Miltenyi) to generate single-cell suspensions. RBC lysis (Biolegend) was performed to remove red blood cells. Tumor samples were harvested and lymphocytes were isolated by dispersing the tumor tissue in 2 ml of PBS, followed by incubation of samples at 37° C. for 15 min with DNase I (Sigma) and Liberase DL (Roche). The suspension was then diluted with MACS buffer and passed through a 70-μm cell strainer to generate a single cell suspension. Cells were prepared in staining buffer (PBS with 2% FBS and 2 mM EDTA) and FcR blocked (clone 2.4G2, BD Biosciences) and stained with indicated primary antibodies for 30 minutes at 4° C.; secondary stains were done for selected markers. Samples were then sorted or fixed and intracellularly stained using a FoxP3 transcription factor kit according to manufacturer's instructions (eBioscience). Cell viability was determined using fixable viability dye (ThermoFisher). For bulk-RNA-seq analyses, the inventors sorted tumor-infiltrating T_{FR} cells based on the co-expression of CXCR5 and GITR^{2,6} (FIG. 6b), a surface marker that distinguishes T_{FH} cells from T_{FR} cells. To accurately assess the expression of intracellularly stored molecules like CTLA-4, the inventors characterized T_{FR} cells based on co-expression of BCL-6 and FOXP3 (FIG. 6c) since cell fixation led to epitope masking of CXCR5 (FIG. 6c, bottom left plot) and GITR. All samples were acquired on a BD FACS Fortessa or sorted on a BD FACS Fusion (both BD Biosciences) and analyzed using FlowJo 10.4.1.

Histology and immunohistochemistry. Deparaffinisation, rehydration, antigen retrieval and IHC staining was carried out using a Dako PT Link Autostainer. Antigen retrieval was performed using the EnVision FLEX Target Retrieval Solution, High pH (Agilent) for all antibodies. The primary antibodies used for IHC include anti-CD8 (pre-diluted, C8/144B, Agilent Dako), anti-CD4 (1:100, 4B12, Agilent Dako), anti-FOXP3 (1:100, ab20034, Abcam), anti-CXCR5 (1:50, D6L3C, CellSignaling), anti-BCL6 (1:30, NCL-L-Bcl6-6-564, Leica), anti-CD31 (pre-diluted product diluted further 1:5, Agilent Dako) and anti-PanCK (AE1/AE3; pre-diluted; Agilent Dako). Primary antibodies were detected using EnVision FLEX HRP (Agilent Dako) and either Rabbit or Mouse Link reagents (Agilent Dako) as appropriate. Chromogenic visualization was completed with either two washes for five minutes in DAB or one wash for thirty minutes in AEC and counterstained with hematoxylin. To analyze multiple markers on single sections, multiplexed IHC staining was performed as described previously. 4 micron tissue sections were stained with anti-PanCK (for NSCLC samples) or anti-CD31 (for melanoma samples) antibodies, visualized using DAB chromogenic substrate and scanned using a ZEISS Axio Scan.Z1 with a 20× air immersion objective. Each immune marker was then visualized using AEC chromogenic substrate and scanned. Between each staining iteration, antigen retrieval was performed, preparing for the subsequent round of staining and denaturing of the preceding antibodies; along with removal of the labile AEC staining in organic solvent (50% ethanol, 2 min; 100% ethanol, 2 min; 100% xylene; 2 min, 100% ethanol, 2 min; and 50% ethanol, 2 min).

For each tissue section the PanCK or CD31 alone image was used as a reference for tiled registration of each staining iteration, using the linear stack alignment with SIFT plugin. Color deconvolution was performed using the “H AEC” vector matrix from the Fiji plugin generating three images representing blue (hematoxylin), red (AEC and DAB) and green (hematoxylin and DAB) staining intensities. These images were inverted, so that higher pixel values represented greater staining intensity. To generate an AEC specific image the “green” pixel intensities were subtracted from the “red” pixel intensities. 8 bit deconvoluted images were then visually inspected to determine a pixel intensity threshold of positive staining for each marker and this value was subtracted from each image to remove non-specific staining. Cell simulation and analysis was then performed using Tissue Studio image analysis software (Definiens). Cells were identified by nucleus detection and cytoplasmic regions were simulated up to 5 μm, per cell protein expression was measured using the mean staining intensity within simulated cell regions.

Bulk-RNA sequencing. Total RNA was purified using a miRNAeasy kit (Qiagen) from human tumor-infiltrating T_{REG} (LIN⁻CD45⁺CD3⁺CD4⁺CXCR5⁻CD127⁻CD25⁺) and T_{FR} (LIN⁻ CD45⁺CD3⁺CD4⁺CXCR5⁺GITR⁺) cells and was quantified as described previously^{45,46}. Cells from mice immunized with either Ovalbumin in Complete Freund's adjuvant (InvivoGen), Ovalbumin in Monophosphoryl Lipid A (InvivoGen) or mock PBS: T_{EFF} (Cd19⁻Cd45⁺Cd3⁺Cd4⁺Cxcr5′Gitr⁻Cd25⁻ Cd62L⁻Cd44⁺), T_{REG} (Cd19⁻Cd45⁺Cd3⁺Cd4⁺Cxcr5⁻Gitr⁺Cd25⁺), T_{FH} (Cd19⁻Cd45⁺Cd3⁺Cd4⁺Cxcr5⁺Gitr⁻) and T_{FR} (Cd19⁻Cd45⁺Cd3⁺Cd4⁺Cxcr5⁺Gitr⁺) were sorted and RNA was purified as described above. RNA-seq libraries were prepared using Smart-seq2 protocol, as previously described⁴⁷. Samples were sequenced using Illumina HiSeq2500 to obtain 50-bp single-end reads. For quality control, steps were included to determine total RNA quality and quantity, the optimal number of PCR pre-amplification cycles, and cDNA fragment size as previously described⁴⁵. Samples that failed quality control, or had a low number of starting cells were eliminated from further sequencing and analysis.

Bulk RNA-seq analysis. Bulk RNA-seq data from human samples were mapped against the hg19 reference using TopHat⁴⁸ (--bowtie 1 -max-multihits 1 -microexon search) with FastQC (v0.11.2), Bowtie⁴⁹ (v1.1.2), Samtools (v0.1.19.0)⁵⁰ and the inventors employed htseq-count -m union -s no -t exon -i gene_name (part of the HTSeq framework, version v0.7.1)⁵¹. Trimmomatic (v0.36) was used to remove adapters⁵². Bulk RNA-seq from mouse samples were mapped against mm10 reference using TopHat (1.4.1) with library-type fr-unstranded parameter. Values throughout are displayed as log₂ TPM (transcripts per million) counts; a value of 1 was added prior to log transformation. To identify genes expressed differentially by various cell types, the inventors performed negative binomial tests for unpaired comparisons by employing the Bioconductor package DESeq2⁵³ (v1.14.1), disabling the default options for independent filtering and Cooks cutoff. The inventors considered genes to be expressed differentially by any comparison when the DESeq2 analysis resulted in a Benjamini-Hochberg-adjusted P value of ≤0.05 and a fold change of at least 2. Euler diagrams were generated using the eulerr package (v5.1.0). Correlations and heatmaps were generated as previously described 46,54,55 Visualizations were generated in ggplot2 using custom scripts. For tSNE analysis, the data frame was filtered to genes with mean≥1 TPM counts expression in at least one condition and visualizations created using the top 500 most variable genes, as calculated in DESeq2⁵³ (v1.16.1); this allowed for unbiased visualization of the Log₂ (TPM counts+1) data, using package Rtsne (v0.13).

Weighted Gene Coexpression Network Analysis. WGCNA was completed in R (v.3.5.0) with the package WGCNA (v1.61) using the TPM data matrix. Well-expressed genes with TPM>=10 in at least one sample, were used in both T_{FR} and T_{REG} data from human. Gene modules were generated using blockwiseModules function (parameters: checkMissingData=TRUE, power=5, TOMType=“signed”, minModuleSize=30, maxBlockSize=13441, mergeCutHeight=0.80). The remaining parameters were as per default in WGCNA. The default ‘grey’ module generated by WGCNA for non-co-expressed genes, was excluded from further analysis. As each module by definition is comprised of highly correlated genes, their combined expression may be usefully summarized by module eigengene (ME) profiles, effectively the first principal component of a given module. A small number of module eigengene profiles may therefore effectively ‘summarize’ the principle patterns within the cellular transcriptome with minimal loss of information. This dimensionality-reduction approach also facilitates correlation of MEs with clinical traits as module-trait relationship matrix. Significance of correlation between this trait and MEs was assessed using linear regression with Benjamini-Hochberg adjustment to correct for multiple testing. The TOMplot was generated using the TOMplot function in WGCNA with default parameters for clustering and color scheme. To visualize co-expression networks were generated in gplots (v3.0.1) using the heatmap2 function, while weighted correlation analysis was completed using WGCNA⁵⁶ (v1.61) from the Log₂ (TPM counts+1) data matrix and the function TOMsimilarityfromExpr (Beta=5) and exportNetworkToCytoscape, weighted=true, threshold=0.05. Highlighted genes were ordered as per the order in the correlation plot. Networks were generated in Gephi (v0.92)^{57,58} using ForceAtlas2 and Noverlap functions. The size and color were scaled according to the Average Degree as calculated in Gephi, while the edge width was scaled according to the WGCNA edge weight value. All analyses were completed in R version in R (v3.5.0).

Meta-analysis of published single-cell RNA-seq studies. The inventors integrated 9 published single-cell RNA-seq datasets^{17,23,59-65} of tumor-infiltrating CD4-expressing T cells with UMAP. The integration was performed using the R package Seurat v3.0. For each dataset, cells that expressed less than 200 genes were considered outliers and discarded. The inventors integrated data from all cohorts using the alignment by ‘anchors’ option in Seurat 3.0. Briefly, the alignment is a computational strategy to “anchor” diverse datasets together, facilitating the integration and comparison of single cell measurements from different technologies and modalities. The “anchors” correspond to similar biological states between datasets. These pairwise correspondences between datasets allows the transformation of datasets into a shared space regardless of the existence of large technical and/or biological divergences. This improved function in Seurat 3.0 allows integration of multiple RNA-seq datasets generated by different platforms⁶⁶. While single cell RNA-seq can be utilized to identify distinct states within a given cell population, it does not offer higher resolution compared to bulk RNA-seq in terms of number of transcripts recovered due to high drop-out rates with single-cell RNA-seq assays, more so with 10×-based assays. The inventors used the FindIntegrationAnchors function to find correspondences across the different study datasets with default parameters (dimensionality=1:30). Furthermore, the inventors used the IntegrateData function to generate a Seurat Object with an integrated and batch-corrected expression matrix. In total, 25,149 cells and 2,000 most variable genes were used for clustering. The inventors used the standard workflow from Seurat, scaling the integrated data, finding relevant components with PCA and visualizing the results with UMAP. The number of relevant components was determined from an elbow plot. UMAP dimensionality reduction and clustering were applied with the following parameters: 2000 genes, 15 principal components, resolution of 0.2, min·dis 0.05 and spread 2. Cells used for the integration were selected from clusters labeled in the original studies as tumor CD4 T cells and from pre-treatment samples when necessary. Cells with expression of CD8B>1 CPM (UMI data) or 10 TPM (Smart-seq2) were filtered out as indicated in Table 1. T_{REG} cells and T_{FR} cells were identified based on criteria defined in Table 1. Only smart-seq2 datasets were used to compare T_{FR} cells from different cancer types.

Single-cell differential expression analysis. Differential expression was calculated with MAST⁶⁷ and SCDE⁶⁸ (v1.99.1) as previously described⁵⁴. For each comparison, the inventors obtained the differentially expressed gene list by taking the union of the gene lists from both the methods using adjusted P<0.05 and log₂ fold change >1 from each method.

Single-cell TCR and transcriptome analysis. Single-cell smart-seq2 data from¹⁷ were re-analyzed (Table 1), using custom scripts to identify αβ chains and showing only cells were both TCR chains were detected, as described previously⁶⁹. Visualizations were completed in ggplot2, Prism (v8.1.1) and custom scripts in TraCer. A cell was considered expanded when both the most highly expressed α and β TCR chain sequences matched other cells with the same stringent criteria. Cells were considered not expanded when α and β TCR productive chain sequences did not match those of any other cells. A cell was considered a T_{REG} cell when the expression of CD4 and FOXP3 were >10 TPM, and lacked expression of CXCR5 and BCL6 (TPM ≤10). A cell was characterized as a T_{FR} cell if expression of CD4 and FOXP3 were >10 TPM and the expression of CXCR5 or BCL6 was >10 TPM. A cell was considered 4-1BB⁺ when the expression of 4-1BB was >10 TPM as indicated in Table 1. Cell-state hierarchy maps were generated using Monocle (v3.0)⁷⁶ and default settings with expressionFamily=negbinomial.size( ), lowerDetectionLimit=0.1 after transformation of TPM counts with relative2abs function as recommended in the manual, including the top 2000 most variable genes identified in Seurat (v3.0) and taking 14 PCs based on the elbow plot. The shared signature was calculated with AddModuleScore function from Seurat after setting the object with default parameters and using the intersection of differentially expressed genes from comparing 4-1BB⁻ T_{REG} cells with three populations: 4-1BB⁺ T_{REG}, clonally-expanded T_{REG} cells sharing their TCRs with T_{FR} and clonally-expanded T_{FR} cells with Benjamini-Hochberg-adjusted P value of <0.05 and a log 2 fold change of 1.Single-cell smart-seq2 data from²³ were utilized to compare the single-cell transcriptome of tumor-infiltrating T_{FR} cells from pre- and post-anti-PD-1 treatment samples.

Hierarchical clustering. Distance between clusters was calculated by obtaining a particular cells location in PCA space (Principal component 1:5) using the function Embeddings from Seurat. The number of principal components was determined from an elbow plot. A distance matrix was calculated (dist function, core R, method=Euclidean) from the PCA matrix and the clustering was performed (hclust function, method=“average”) in R and generated from the distance matrix. Function colored bars from the WGCNA package was used to annotate different groups in the dendrogram.

Single-cell transcriptome analysis of primary tumor tissue and metastatic tumor-infiltrated lymph nodes. Human T cells from 2 HNSCC patients (primary tumor tissue and metastatic tumor-infiltrated lymph nodes) were isolated and prepared for flow-cytometric sorting from cryopreserved tumor tissue as described above. CD4⁺ T_{H} cells (CXCR5⁺GITR⁻ and CXCR5⁻CD25⁻), T_{REG} cells (CD4⁺CXCR5⁻ CD25⁺CD127^{lo}), T_{FR} cells (CD4⁺CXCR5⁺GITR⁺) and CD8⁺CD69⁺ cells were sorted and cDNA libraries were constructed using the standard 10× sequencing protocol. A total of n=9,562 (n=4,975 from metastatic tumor-infiltrated lymph node, n=4,589 from primary tumor tissue) cells were sequenced and cells with less than 200 and more than 5,000 expressed genes, less than 15,000 counts, and more than 10% of mitochondrial counts were filtered out. For clustering with Seurat (3.0) the inventors used 17 PCs from a set of highly variable genes (n=609) taking 30% of the variance after filtering out genes with mean expression less than 0.1 and removing TCR genes. TCR analysis: clonotype output (clonotypes and filtered contig annotation) from Cell Ranger for tumor and lymph node libraries were re-calculated (matching sequences were assigned the same clonotype id) and the overlap between cluster 1 and 6 was determined with these ‘aggregated’ tables. Gene Set Enrichment analysis: the Log 2 fold change was used as ranking metric and enrichment was calculated for each list. fgsea (1.13.0) in R with default parameters was used to calculate the enrichment and create GSEA plots. Monocle (2.99.1) was used to generate the trajectory plots, reduction_method=DDRTree for the dimensional reduction taking 15 principal components. Hierarchical clustering was performed as stated above with 20 PCs.

Quantification and statistical analysis. The number of subjects, samples or mice/group, replication in independent experiments, and statistical tests can be found in the figure legends. Details on quality control, sample elimination and displayed data are stated the method details and figure legends. Sample sizes were chosen based on published studies to ensure sufficient numbers of mice in each group enabling reliable statistical testing and accounting for variability. Sample sizes are indicated in Figure legends. Mice, which didn't develop any tumors by 10 after inoculation were excluded from analyses, prior to any therapeutic intervention. RNA-seq samples that didn't pass quality control weren't included in the analyses. Experiments were reliably reproduced in independent experiments at least twice. Animals of same sex and age were randomly assigned to experimental groups Statistical analyses were performed with Graph Pad Prism 8 and statistical tests used are indicated in the figure legends and experimental model and subject details.

### Example 2. Depletion of T_{FR} but not T_{regs} to Prevent Severe Immune-Related Adverse Events (irAEs)

Immune checkpoint blockade (ICB) targeting CTLA-4 or PD-1 can lead to dramatic, long-lasting responses; nonetheless, fewer than 30% of patients respond to monotherapy with either agent. While anti-CTLA-4 therapy is believed to deplete T regulatory (T_{REG}) cells, anti-PD-1 blocking antibodies are thought to primarily activate CD8⁺ T cells. Combination therapy, though more effective, causes more frequent and severe immune-related adverse events (irAEs), potentially caused by undirected anti-CTLA-4-mediated TREG cell depletion and subsequent uninhibited anti-PD-1-mediated activation of effector T cells. As described hereinabove, a novel population of T cells, follicular regulatory T cells (T_{FR}), are a district population of regulatory T cells that inhibit CD8 T cells. As shows in the example above, by increasing the abundance of T_{FR} cells, anti-PD-1 therapy not only facilitates, but also dampens anti-tumor immunity. Conditional knockout of T_{FR} cells or depletion of T_{FR} cells with anti-CTLA-4 antibody prior to anti-PD-1 treatment, improved tumor control in mice. In a large melanoma cohort, sequential ICB (anti-CTLA-4 prior to anti-PD-1 therapy) was associated with better long-term survival outcomes when compared to concomitant combination therapy or monotherapy with either agent, highlighting the clinical benefit of sequential ICB to render tumors responsive to anti-PD1 therapy. Translating the benefits of ICB to a broader patient cohort while minimizing irAEs requires a deeper understanding of its cellular targets and specific mode of action. A better understanding of the tumor microenvironmental cues that trigger T_{REG} to T_{FR} differentiation, how T_{FR} cells inhibit anti-tumor immunity and whether targeting them more specifically (without affecting T_{REG} cells) to decrease irAEs while maintaining treatment efficacy, is included herein.

Adoptive transfer studies can be used in conjunction with TCR trajectory analyses to show intratumoral T_{REG} to T_{FR} conversion. The factors driving this differentiation step can be determined using large-scale, high-resolution smart-seq3 single-cell RNA-seq and single-cell ATAC-seq of human tumor-infiltrating T_{REG} and T_{FR} cells from three common cancer types (NSCLC, HNSCC and melanoma). The transcriptional profiles and enhancer landscapes (PageRank analysis) of T_{REG} and T_{FR} cells to identify common are compared (shared between cell types), as well as unique, cell type specific molecules and transcription factors (TFs) potentially driving differentiation. Protein expression and DNA binding studies can be used to verify the most significant TFs using micro-scaled ChIP assays, and test functional significance of the top ‘candidate’ molecule in murine tumor models. Novel immunotherapy target to selectively deplete T_{FR} cells. Given that anti-CTLA-4 antibodies non-specifically deplete both, T_{REG} and T_{FR} cells, potentially causing irAEs, targeting IL1R2, a surface molecule specifically expressed on T_{FR} cells, can be used to study the anti-tumor effects of T_{FR} cell-deficient mice and in the model systems described in Example 1. Antibody clones can be generated and studied using the Beacon platform (Berkeley Lights), select clones with heightened ADCC activity are isolated and their functionality assessed in multiple murine tumor models.

Molecular mechanisms driving augmented anti-tumor immunity. Having established that mice that are selectively deficient in T_{FR} cells (without affecting the T_{REG} cell compartment) display augmented anti-tumor immunity, the molecular mechanisms driving this effect can be determined. For example, whether T_{FR} cells, and not T_{REG} cells, impact the priming or activity of tumor-antigen specific cytotoxic T cells and whether T_{FR} cells induce transcriptomic changes in CTLs and other tumor-infiltrating lymphocyte compartments can be assessed as outlined in Example 1. Moreover, how T_{FR} cells curtail anti-PD-1 treatment efficacy and mediate irAEs can be studied by specific T_{FR} cell depletion (anti-IL1R2, genetic depletion), which minimizes irAEs when compared to undirected depletion of all FoxP3⁺ cells (T_{REG} and T_{FR}) (anti-CTLA-4, FoxP3^{DTR}) while maintaining treatment efficacy of combination therapy, thus paving the way for novel combination therapies (anti-IL1R2+anti-PD-1).

Additional transcriptomic differences and similarities between intratumoral T_{REG} and T_{FR} cells to further discern the roles they play in anti-tumor immunity using cell type-specific enhancer regions to uncover binding sites for TFs that play a role in maintenance, functionality or differentiation. As cell numbers for human analyses are likely to be limited, single-cell ATAC-seq and micro-scaled ChIP-seq assays can be used. Coupling high-resolution single-cell RNA-seq and single-cell ATAC-seq with ChIP-seq has several benefits: (i) Single-cell RNA-seq to identify cell type-specific TFs. (ii) TFs drive lineage differentiation and cell-specific functions can be traced back by assessing the enhancer landscape. During cell fate specification, lineage-driving TFs leave a ‘footprint’ on the cell's enhancer landscape, often accompanied by increased chromatin accessibility (measured by ATAC-Seq). (iii) Genes being bound and thus affected by the identified TFs are defined by utilizing micro-scaled ChIP-seq.

Diverging roles for T_{REG} and T_{FR} cells in anti-tumor immunity. In the example above, T_{REG} cells have been shown to differentiate into PD-1 expressing follicular regulatory T cells (T_{FR}) that restrain germinal center responses, impede humoral immunity towards self-antigens and display heightened suppressive capacity when compared to T_{REG}.

As shown hereinabove, T_{FR} cells are characterized by their joint expression of, e.g., the surface molecules Cxcr5 and Gitr, or by their co-expression of the transcription factors FoxP3 and Bcl6. While it is well-established that T_{REG} cells can inhibit anti-tumor immunity, few studies have examined the potential effects of ICB on this cell compartment. Moreover, T_{FR} cells, their functional role in cancer, and their responsiveness to immunotherapy drugs have been completely disregarded so far.

It was now possible to investigate if T_{REG} and T_{FR} cells accumulate in parallel in the tumor microenvironment (TME) as a means of effective immune evasion. As shown above, tumor-infiltrating T_{FR} cells were highly prevalent in a variety of different cancer types. When compared to T_{REG} cells, T_{FR} cells showed superior suppressive capacity, enhanced proliferative capacity and Bcl6-dependent in vivo persistence. Recent data demonstrate that tumor-infiltrating T_{REG} cells potently recognize tumor (neo)antigens and, upon antigen-encounter, undergo clonal expansion. RNA-seq, TCR-seq and trajectory analyses of tumor-infiltrating T_{REG} and T_{FR} cells show that the tumor microenvironment (TME) is initially infiltrated by a large and highly diverse pool of bystander (i.e., not TAA-specific) T_{REG} cells, and a smaller pool of TAA-specific T_{REG} clones, which are poised for differentiation into tissue resident T_{FR} cells. In a genetic murine model permitting selective T_{FR} cell depletion, it was found that T_{FR} cells inhibit anti-tumor immunity to a similar degree as depletion of all FoxP3-expressing cells (T_{REG} and T_{FR}). Thus, selective depletion of T_{FR} cells is clinically beneficial and will cause fewer irAEs. Thus, it is important to use therapeutic strategies to specifically deplete T_{FR} cells without affecting the bystander T_{REG} cell compartment. Compared to T_{REG} cells and CD8⁺ TILs, T_{FR} cells expressed the highest levels of CTLA-4 and PD-1. As such, T_{FR} cells are highly responsive to ICB targeting these molecules. Accordingly, while anti-CTLA-4 treatment preferentially depleted T_{FR} cells in tumor tissues, the inventors observed substantial depletion of bystander T_{REG} cells, potentially causative of irAEs found in the clinical setting. These data not only demonstrate that anti-PD-1 therapy can not only facilitate, but also dampen anti-tumor immune attack by activating highly suppressive T_{FR} cells in tumor tissues, but also highlight the clinical benefit of sequential ICB. These findings provide critical insights into how anti-CTLA-4 and anti-PD-1 therapies mediate their function and point to a potential cause of exacerbated irAEs observed upon combination therapy. Thus, these support further study of the diverging roles of T_{REG} and T_{FR} cells in cancer and the molecular mechanisms driving intratumoral T_{REG} to T_{FR} conversion. The observed effects of specific T_{FR} cell depletion can be recapitulated by generating antibodies against IL1R2, a novel immunotherapy target to assess how T_{FR} cells impact the priming or activity of tumor antigen-specific CTLs by profiling T_{FR} cell-induced changed in their transcriptomic signatures and will discern the relative contribution of T_{REG} and T_{FR} cells onto anti-tumor immunity and anti-PD-1 treatment efficacy and irAEs, in the same manner as described to anti-CLTA4 hereinabove.

The present invention can also be used to clarify the diverging roles that T_{REG} and T_{FR} cells play in anti-tumor immunity and identifying key molecular players and pathways driving the development and functionality of highly suppressive T_{FR} cells in tumor tissues will inform the discovery of novel immunotherapy drug targets to treat cancer. The factors that trigger developmental and functional changes in tumor-infiltrating T_{REG} and T_{FR} cells can also be determined using this model system. Further, pre-clinical and clinical findings on T_{FR} cells to further improve immunotherapy efficiency and to translate its benefits to a larger group of patients can also be determined using the present model.

Further, it is possible to combine genetic and drug-related strategies to unravel the diverse roles T_{REG} and T_{FR} cells play in anti-tumor immunity and improve current treatment regimens as well as evaluate its effects on priming and activity of tumor antigen-specific CTLs and generate and test a novel immunotherapy antibody to mirror the anti-tumor effects of specific T_{FR} cell depletion.

Further evaluation of the highly suppressive intratumoral T_{FR} cells disclosed herein can be used to improve anti-tumor immunity and ICB treatment efficacy. It can also be used enhance the understanding of cellular and molecular mechanisms facilitating efficacious anti-tumor reactions and may lead to more specific therapies as well as identify novel targets for immunotherapy and therapeutic interventions.

Sequential ICB treatment is associated with better survival outcomes.

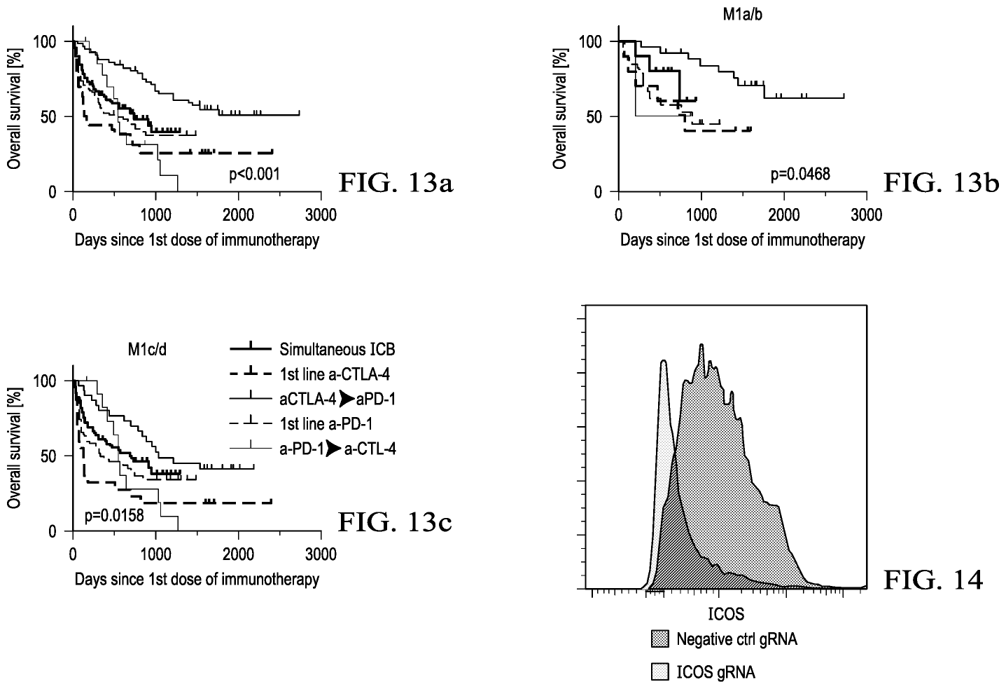

To test the clinical significance of sequential ICB treatment, the inventors retrospectively assessed the survival outcomes of patients with inoperable melanoma (n=271), who were, based on their treatment regimens, stratified into 5 groups: 1st line anti-CTLA-4, 1st line anti-PD-1, simultaneous combination therapy, sequential therapy with anti-CTLA-4 followed by anti-PD-1 at progression and vice versa. Sequential treatment with anti-CTLA-4 followed by anti-PD-1 was associated with better long-term overall survival (OS) outcomes when compared to the 4 other groups (p<0.001) (FIGS. 13a-13c). It has to be noted though that patients receiving simultaneous ICB therapy exhibited a more advanced disease prior to treatment initiation (higher proportion with AJCC 8 stage M1c and M1d (n=75) than patients on 1st line anti-PD-1 (n=70) or 1st line anti-CTLA-4 (n=52), (FIGS. 13a-13c), likely contributing to their poor OS outcomes. However, the advantageous effect of anti-CTLA-4 followed by anti-PD-1 therapy was preserved in patients with M1a/b and M1c/d, respectively, indicating that this treatment regimen is clinically beneficial. Differences in BRAF status did not affect ICB treatment outcomes (data not shown).

Crucially and in-line with our findings in mouse models (FIGS. 4b to 4d, example 1), when compared to monotherapy with anti-PD1, sequential treatment with anti-CTLA-4 (likely to deplete T_{FR} cells in the tumor) followed by anti-PD-1 was associated with significantly better survival outcomes (p=0.0003).

Identify molecular programs driving T_{REG} to T_{FR} differentiation in tumor tissue.

High-resolution smart-seq3 single-cell RNA-seq has a significantly higher (2-5 fold) gene coverage than previous (smart-seq2 and 10×) methods and can thus reveal currently unknown patterns of gene expression. The high-resolution smart-seq3 single-cell RNA-seq sequencing platform can be used to fully characterize the transcriptomic signatures of tumor-infiltrating T_{REG} (CD4⁺CD127^{lo}CD25⁺CXCR5⁻) and T_{FR} (CD4⁺CXCR5⁺GITR⁺) cells. The results will define genes and transcription factors (TFs) that are pivotal for the heightened suppressive capacity of T_{FR} cells (see Example 1) and for their differentiation. Single-cell RNA-seq, single-cell ATAC-seq and micro-scaled ChIP-seq assays will allow us to bypass potentially low cell numbers, identify novel genes and TFs, permit assessing the enhancer landscape of intratumoral T_{REG} and T_{FR} cells, and delineate potential alterations in gene expression by bound TFs.

To identify genes and TFs specific to human tumor-infiltrating T_{REG} and T_{FR} cells, high-resolution single-cell RNA-seq in purified (˜500 T_{REG} or T_{FR}) cells isolated can be performed from 3 human cancer types (NSCLC, HNSCC and melanoma; n=8 samples/tumor type. Human tumor samples will be obtained from studies at a tertiary center in the UK that is actively recruiting patients (˜20 cases/month). This will allow for the efficient characterization of the transcriptomic signatures of tumor-infiltrating T_{REG} and T_{FR} cells of different cancer types. Additionally, patient-matched T_{REG} and T_{FR} cells can be sorted for single-cell ATAC-seq and enhancer profiling. Comparing the enhancer landscapes of tumor-infiltrating T_{REG} cells with that of T_{FR} cells will determine which enhancers are cell type specific. Subsequently, enhancers specific to each cell type, and then employ PageRank16 analysis to predict TFs can be used to establish the enhancer landscape of T_{FR} cells; such TFs are likely to be pivotal for T_{FR} cell differentiation. Lastly, the TFs identified in the previous steps can be subjected to in micro-scaled ChIP assays17 to determine genes that are being bound by these TFs.

Functional studies: Demonstrating that a given TF, such as BCL6, is critical for the differentiation of T_{REG} cells into T_{FR} cells in tumors requires assessing the impact of manipulating the expression levels of that TF by depletion or overexpression in vivo. Such mechanistic studies are best performed in murine tumor models, where the abundance and function of T_{REG} and T_{FR} cells can be easily assessed. To test the functional role of the top ‘candidate’ TF or molecule emerging from our studies (conserved between human and mouse), CRISPR-Cas9-mediated depletion of the ‘candidate’ molecule in T_{REG} cells can be used to assess the impact of their differentiation into T_{FR} cells in murine tumor models. Knockdown efficiency can be verified at the transcript level and at the protein level, if suitable antibodies are available. Performing CRISPR-Cas9 based gene depletion in primary T cells, and show feasibility of depleting ICOS in T cells can also be used to adoptively transfer T_{REG} cells that are sufficient or deficient in the ‘candidate’ molecule into B16F10-OVA tumor-bearing recipient (congenic C57BL/6J or Rag1^{-/-}) mice. Knockout mice for the ‘candidate’ molecule can be used to purify and adoptively transfer T_{REG} cells deficient with the ‘candidate’ molecule. Tumor growth over the course of the experiment and sacrifice mice at day 13-18 after adoptive transfer and assess alterations in T_{FR} cell frequency.

Finally, an unbiased “genomics-based” approach I am taking is likely to identify several key players driving T_{REG} to T_{FR} differentiation and cell functionality, that will fuel additional important hypotheses and strands of research. FIG. 14 shows CRISPR-based deletion of target gene (ICOS) in primary CD4+ T cells. ICOS protein expression following ICOS CRISPRi guide RNA (yellow color) or control guide RNA (blue color) transduction in CD4+ T cells.

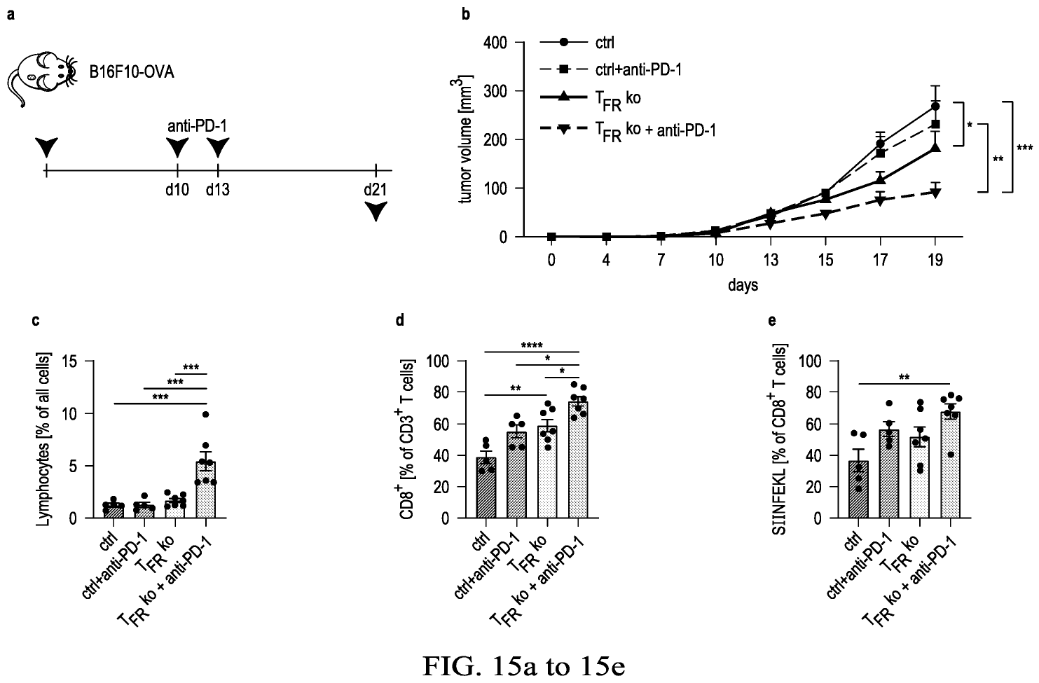

FIGS. 15a and 15e show Control mice or FoxP3^{YFPcre}×Bcl6^{fl/fl} (T_{FR} ko) mice were s.c. inoculated with B16F10-OVA cells and treated with isotype control or anti-PD-1 Abs at indicated time points, Tumor volume (FIG. 15a) and cell frequencies (FIG. 15b) of mice treated as indicated n=9-11 mice/group (FIG. 15a) and n=5-7 mice/group (FIG. 15b). FIGS. 15c to 15 e area graphs with the results. One-way ANOVA was used to compare the mean of each column with the mean of each other column followed by Dunnett's test.

The data in FIGS. 15a-15e demonstrate that T_{FR} cells inhibit anti-tumor immunity, by impeding CD8+ T cell activity or priming, and curtail anti-PD-1 treatment efficacy, further corroborating the results hereinabove. Moreover, these data show that combination therapy, where T_{FR} cells are being depleted prior to initiation of anti-PD-1 therapy, facilitates efficacious anti-tumor immunity.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. De Simone, M. et al. Transcriptional Landscape of Human Tissue Lymphocytes Unveils Uniqueness of Tumor-Infiltrating T Regulatory Cells. Immunity 45, 1135-1147 (2016).
2. Linterman, M. A. et al. Foxp3+ follicular regulatory T cells control the germinal center response. Nat. Med. 17, 975-982 (2011).
3. Sage, P. T. et al. Suppression by T FR cells leads to durable and selective inhibition of B cell effector function. Nat. Immunol. 17, 1436-1446 (2016).
4. Sage, P. T., Paterson, A. M., Lovitch, S. B. & Sharpe, A. H. The coinhibitory receptor CTLA-4 controls B cell responses by modulating T follicular helper, T follicular regulatory, and T regulatory cells. Immunity 41, 1026-1039 (2014).
5. Sage, P. T., Alvarez, D., Godec, J., Von Andrian, U. H. & Sharpe, A. H. Circulating T follicular regulatory and helper cells have memory-like properties. J. Clin. Invest. 124, 5191-5204 (2014).
6. Sage, P. T., Francisco, L. M., Carman, C. V. & Sharpe, A. H. The receptor PD-1 controls follicular regulatory T cells in the lymph nodes and blood. Nat. Immunol. 14, 152-161 (2013).
7. Vanderleyden, I. et al. Follicular Regulatory T Cells Can Access the Germinal Center Independently of CXCR5. Cell Rep. 30, 611-619.e4 (2020).
8. Brenna, E. et al. CD4+T Follicular Helper Cells in Human Tonsils and Blood Are Clonally Convergent but Divergent from Non-Tfh CD4+ Cells. Cell Rep. 30, 137-152.e5 (2020).
9. Roma), V. C. et al. Human blood T fr cells are indicators of ongoing humoral activity not fully licensed with suppressive function. Sci. Immunol. 2, eaan1487 (2017).
10. Ritvo, P.-G. G. et al. T fr cells lack IL-2Ra but express decoy IL-1R2 and IL-1Ra and suppress the IL-1-dependent activation of T fh cells. Sci. Immunol. 2, eaan0368 (2017).
11. Botta, D. et al. Dynamic regulation of T follicular regulatory cell responses by interleukin 2 during influenza infection. Nat. Immunol. 18, 1249-1260 (2017).
12. Chung, Y. et al. Follicular regulatory T cells expressing Foxp3 and Bcl-6 suppress germinal center reactions. 17, (2011).
13. Chen, X. et al. Cutting edge: expression of TNFR2 defines a maximally suppressive subset of mouse CD4+CD25+FoxP3+ T regulatory cells: applicability to tumor-infiltrating T regulatory cells. J. Immunol. 180, 6467-6471 (2008).
14. Huang, C.-T. et al. Role of LAG-3 in regulatory T cells. Immunity 21, 503-513 (2004).
15. Joller, N. et al. Treg cells expressing the coinhibitory molecule TIGIT selectively inhibit proinflammatory Th1 and Th17 cell responses. Immunity 40, 569-581 (2014).
16. Hayatsu, N. et al. Analyses of a Mutant Foxp3 Allele Reveal BATF as a Critical Transcription Factor in the Differentiation and Accumulation of Tissue Regulatory T Cells. Immunity 47, 268-283.e9 (2017).
17. Guo, X. et al. Global characterization of T cells in non-small-cell lung cancer by single-cell sequencing. 24, (2018).
18. Plitas, G. et al. Regulatory T Cells Exhibit Distinct Features in Human Breast Cancer. Immunity 45, 1122-1134 (2016).
19. Miller, B. C. et al. Subsets of exhausted CD8+ T cells differentially mediate tumor control and respond to checkpoint blockade. Nat. Immunol. 20, 326-336 (2019).
20. Im, S. J. et al. Defining CD8+ T cells that provide the proliferative burst after PD-1 therapy. Nature 537, 417-421 (2016).
21. Utzschneider, D. T. et al. T Cell Factor 1-Expressing Memory-like CD8+ T Cells Sustain the Immune Response to Chronic Viral Infections. Immunity 45, 415-427 (2016).
22. Siddiqui, I. et al. Intratumoral Tcf1+PD-1+CD8+ T Cells with Stem-like Properties Promote Tumor Control in Response to Vaccination and Checkpoint Blockade Immunotherapy. Immunity 50, 1-17 (2019).
23. Sade-Feldman, M. et al. Defining T Cell States Associated with Response to Checkpoint Immunotherapy in Melanoma. Cell 175, 998-1013.e20 (2018).
24. Ahmadzadeh, M. et al. Tumor-infiltrating human CD4+ regulatory T cells display a distinct TCR repertoire and exhibit tumor and neoantigen reactivity. Sci. Immunol. 4, eaao4310 (2019).
25. Xu, L. et al. The Kinase mTORC1 Promotes the Generation and Suppressive Function of Follicular Regulatory T Cells. Immunity 47, 538-551.e5 (2017).
26. Kniemeyer, O., Brakhage, A. A., Ferreira, F., Wallner, M. & Sawitzki, B. Regulatory T Cell Specificity Directs Tolerance versus Allergy against Aeroantigens in Humans. Cell 167, 1067-1078.e16 (2016).
27. Szabo, P. A., Miron, M. & Farber, D. L. Location, location, location: Tissue resident memory T cells in mice and humans. Sci. Immunol. 4, eaas9673 (2019).
28. Akkaya, B. et al. Regulatory T cells mediate specific suppression by depleting peptide-MHC class II from dendritic cells. Nat. Immunol. 20, 218-231 (2019).
29. Binnewies, M. et al. Unleashing Type-2 Dendritic Cells to Drive Protective Antitumor CD4+ T Cell Immunity. Cell 177, 556-571.e16 (2019).
30. Pace, L. et al. Regulatory T cells increase the avidity of primary CD8+ T cell responses and promote memory. Science (80-.). 338, 532-536 (2012).
31. Page, N. et al. Expression of the DNA-Binding Factor TOX Promotes the Encephalitogenic Potential of Microbe-Induced Autoreactive CD8+ T Cells. Immunity 48, 937-950.e8 (2018).
32. Scott, A. C. et al. TOX is a critical regulator of tumour-specific T cell differentiation. Nature (2019) doi:10.1038/s41586-019-1324-y.
33. Fu, W. et al. Deficiency in T follicular regulatory cells promotes autoimmunity. J. Exp. Med. 215, 815-825 (2018).
34. Helmink, B. A. et al. B cells and tertiary lymphoid structures promote immunotherapy response. Nature (2020) doi:10.1038/s41586-019-1922-8.
35. Cabrita, R. et al. Tertiary lymphoid structures improve immunotherapy and survival in melanoma. Nature (2020) doi:10.1038/s41586-019-1914-8.
36. Petitprez, F. et al. B cells are associated with survival and immunotherapy response in sarcoma. Nature (2020) doi:10.1038/s41586-019-1906-8.
37. Simpson, T. R. et al. Fc-dependent depletion of tumor-infiltrating regulatory T cells co-defines the efficacy of anti-CTLA-4 therapy against melanoma. J. Exp. Med. 210, 1695-1710 (2013).
38. Kleffel, S. et al. Melanoma Cell-Intrinsic PD-1 Receptor Functions Promote Tumor Growth. Cell 162, 1242-1256 (2015).
39. Wolchok, J. D. et al. Overall Survival with Combined Nivolumab and Ipilimumab in Advanced Melanoma. N. Engl. J. Med. NEJMoa1709684 (2017) doi:10.1056/NEJMoa1709684.
40. Champiat, S. et al. Hyperprogressive disease is a new pattern of progression in cancer patients treated by anti-PD-1/PD-L1. Clin. Cancer Res. 23, 1920-1928 (2017).
41. Knorr, D. A. & Ravetch, J. V. Immunotherapy and hyperprogression: Unwanted outcomes, unclear mechanism. Clin. Cancer Res. 25, 904-906 (2019).
42. Locci, M. et al. Human circulating PD-1+CXCR3-CXCR5+ memory Tfh cells are highly functional and correlate with broadly neutralizing HIV antibody responses. Immunity 39, 758-769 (2013).
43. Juneja, V. R. et al. PD-L1 on tumor cells is sufficient for immune evasion in immunogenic tumors and inhibits CD8 T cell cytotoxicity. J. Exp. Med. 214, 895-904 (2017).
44. Webster, K. E. et al. In vivo expansion of t reg cells with il-2-mab complexes: induction of resistance to eae and long-term acceptance of islet allografts without immunosuppression. J. Exp. Med. 206, 751-760 (2009).
45. Ganesan, A. P. et al. Tissue-resident memory features are linked to the magnitude of cytotoxic T cell responses in human lung cancer. Nat. Immunol. 18, 940-950 (2017).
46. Engel, I. et al. Innate-like functions of natural killer T cell subsets result from highly divergent gene programs. Nat. Immunol. 17, 728-739 (2016).
47. Picelli, S. et al. Full-length RNA-seq from single cells using Smart-seq2. Nat. Protoc. 9, 171-181 (2014).
48. Trapnell, C., Pachter, L. & Salzberg, S. L. TopHat: discovering splice junctions with RNA-Seq. Bioinformatics 25, 1105-1111 (2009).
49. Langmead, B., Trapnell, C., Pop, M. & Salzberg, S. L. Ultrafast and memory-efficient alignment of short DNA sequences to the human genome. Genome Biol. 10, R25 (2009).
50. Li, H. & Durbin, R. Fast and accurate short read alignment with Burrows-Wheeler transform. Bioinformatics 25, 1754-1760 (2009).
51. Anders, S., Pyl, P. T. & Huber, W. HTSeq—a Python framework to work with high-throughput sequencing data. Bioinformatics 31, 166-169 (2015).
52. Bolger, A. M., Lohse, M. & Usadel, B. Trimmomatic: a flexible trimmer for Illumina sequence data. Bioinformatics 30, 2114-2120 (2014).
53. Love, M. I., Huber, W. & Anders, S. Moderated estimation of fold change and dispersion for RNA-seq data with DESeq2. Genome Biol. 15, 550 (2014).
54. Patil, V. S. et al. Precursors of human CD4+ cytotoxic T lymphocytes identified by single-cell transcriptome analysis. Sci. Immunol. 3, 8664 (2018).
55. Ganesan, A.-P. et al. Tissue-resident memory features are linked to the magnitude of cytotoxic T cell responses in human lung cancer. Nat. Immunol. 18, 940-950 (2017).
56. Langfelder, P. & Horvath, S. WGCNA: an R package for weighted correlation network analysis. BMC Bioinformatics 9, 559 (2008).
57. Mellone, M. et al. Induction of fibroblast senescence generates a non-fibrogenic myofibroblast phenotype that differentially impacts on cancer prognosis. Aging (Albany. N.Y.). 9, 114-132 (2016).
58. Ottensmeier, C. H. et al. Upregulated Glucose Metabolism Correlates Inversely with CD8+ T-cell Infiltration and Survival in Squamous Cell Carcinoma. Cancer Res. 76, 4136-4148 (2016).
59. Zheng, C. et al. Landscape of Infiltrating T Cells in Liver Cancer Revealed by Single-Cell Sequencing Resource Landscape of Infiltrating T Cells in Liver Cancer Revealed by Single-Cell Sequencing. Cell 169, 1342-1356.e16 (2017).
60. Zhang, L. et al. Lineage tracking reveals dynamic relationships of T cells in colorectal cancer. Nature 564, 268-272 (2018).
61. Savas, P. et al. Single-cell profiling of breast cancer T cells reveals a tissue-resident memory subset associated with improved prognosis. Nat. Med. 24, 986-993 (2018).
62. Lambrechts, D. et al. Phenotype molding of stromal cells in the lung tumor microenvironment. Nat. Med. 24, 1277-1289 (2018).
63. Puram, S. V. et al. Single-Cell Transcriptomic Analysis of Primary and Metastatic Tumor Ecosystems in Head and Neck Cancer. Cell 171, 1611-1624.e24 (2017).
64. Jerby-Arnon, L. et al. A Cancer Cell Program Promotes T Cell Exclusion and Resistance to Checkpoint Blockade. Cell 175, 984-997.e24 (2018).
65. Li, H. et al. Dysfunctional CD8 T Cells Form a Proliferative, Dynamically Regulated Compartment within Human Melanoma. Cell 176, 775-789.e18 (2019).
66. Stuart, T. et al. Comprehensive Integration of Single-Cell Data. Cell 177, 1888-1902.e21 (2019).
67. Finak, G. et al. MAST: A flexible statistical framework for assessing transcriptional changes and characterizing heterogeneity in single-cell RNA sequencing data. Genome Biol. 16, 278 (2015).
68. Kharchenko, P. V., Silberstein, L. & Scadden, D. T. Bayesian approach to single-cell differential expression analysis. Nat. Methods 11, 740-742 (2014).
69. Patil, V. S. et al. Precursors of human CD4+ cytotoxic T lymphocytes identified by single-cell transcriptome analysis. Sci. Immunol. 3, eaan8664 (2018).
70. Trapnell, C. et al. The dynamics and regulators of cell fate decisions are revealed by pseudotemporal ordering of single cells. Nat. Biotechnol. 32, 381-386 (2014).

What is claimed is:

1. A method of detecting follicular regulatory T cells (T_{FR}) comprising:
obtaining a biological sample from a subject; and
detecting whether T_{FR} cells are present or increased in the biological sample by contacting the biological sample with antibodies that detect CD3⁺CD4⁺FOXP3⁺BCL6⁺ T cells CD3⁺CD4⁺CXCR5⁺GITR⁺ T cells, or both, when compared to a healthy subject, and detecting the increase of T_{FR} cells in the biological sample.

2. The method of claim 0, further comprising detecting the presence or a high level of expression of at least one of: PD-1, CTLA-4, 4-1BB, ICOS, Tox, Ki67, or TCF1 on the T_{FR}.

3. The method of claim 0, wherein the step of detecting is measuring mRNA, protein, or both.

4. The method of claim 0, wherein the T_{FR} cells are defined further as CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells or any combination thereof.

5. The method of claim 0, wherein the T_{FR} cells are not LIN⁻CD45⁺CD3⁺CD4⁺CXCR5⁻ FOXP3⁺BCL6⁻PD-1⁻ cells.

6. The method of claim 0, wherein the biological sample is a cancer tissue.

7. The method of claim 0, wherein the biological sample is a tumor sample selected from a colorectal, a melanoma, a lung, a liver, a head and neck, or a breast cancer issue.

8. The method of claim 0, wherein the biological sample is obtained from a subject suspected of having an immune reactive adverse effect (IRAE).

9. The method of claim 0, wherein the T_{FR} cells are PD-1^{high}.

10. The method of claim 0, wherein the biological sample is contacted with antibodies that detect CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺ BCL6⁺GITR⁺ T cells, or any combination thereof.

11. The method of claim 0, wherein the increase of T_{FR} cells is detected in the biological sample as compared to a healthy subject.

12. A method of diagnosing and treating a cancer in a patient, the method comprising the steps of:
determining whether the patient has an increase in PD-1 expressing follicular regulatory T (T_{FR}) cells in or about the cancer by:
obtaining or having obtained a biological sample from the patient;
performing or having performed an assay on the biological sample to determine if the patient has an increase in PD-1 expressing T_{FR} cells, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof, when compared to a reference level generated for specific tumor types or a healthy patient by;
identifying that the patient has an increase in T_{FR} cells that will limit the effectiveness of anti-PD-1 cancer therapy; and
if the patient has T_{FR} cells or shows an increase in T_{FR} cells, then internally administering a selective T_{FR} cell depleting therapy to the patient, and
if the patient does not have T_{FR} cells, an increase in the T_{FR} cells, or if the T_{FR} cells have been depleted by administering a T_{FR} cell depleting therapy to the patient, then administering anti-PD-1 therapy to the patient in an amount sufficient to treat the cancer,
wherein a failure to control cancer growth or an immune related adverse effects (irAE) is lower following the depletion of FoxP3-expressing regulatory T (T_{REG}) cells and the T_{FR} cells in the patient.

13. The method of claim 12, wherein the presence of T_{FR} cells is determined in a tumor biopsy.

14. The method of claim 12, wherein the step of detecting is measuring mRNA, protein, or both.

15. The method of claim 12, wherein the selective T_{FR} cell depleting therapy is at least one of anti-CTLA-4, anti-IL1R2, anti-4-1BB, anti-ICOS, anti-GITR, anti-OX40, anti-TNFR2, or anti-CCR8 therapy, or other targets specifically expressed or enriched on T_{FR} cells when compared to T_{REG} cells and other T cell populations.

16. The method of claim 12, wherein the cancer is selected from a colorectal, a melanoma, a lung, a liver, a head and neck, and a breast cancer.

17. The method of claim 12, wherein the T_{FR} cells express one or more of the following markers: FOXP3, GITR, CTLA-4, 4-1BB, ICOS, Tox, Ki67, and TCF1.

18. The method of claim 12, wherein the presence of T_{FR} cells is further determined by measuring the expression of one or more genes selected from Tnfrsf1b, Lag3, Tigit, Batf, Illr2, Ccr8, Pdcd1, Tox, CCR8, TNFRSF1B, DUSP14, CLP1.

19. The method of claim 12, wherein the selective T_{FR} cell depleting therapy does not reduce or eliminate T_{REGS}.

20. A method for treating a patient suffering from a cancer susceptible to anti-PD-1 therapy, the method comprising the steps of:
determining whether the patient has an increase in PD-1 expressing follicular regulatory T (T_{FR}) cells in or about the cancer, when compared to a reference level generated for specific tumor types or a healthy patient by:
obtaining or having obtained a biological sample from the patient; and
performing or having performed an assay on the biological sample to determine if the patient has PD-1 expressing T_{FR} cells; and
if the patient has the PD-1 expressing T_{FR} cells, then administering a PD-1 expressing T_{FR} depleting therapy to the patient, and
if the patient does not have the T_{FR} cells or if the T_{FR} cells have been depleted by administering a selective T_{FR} cell depleting therapy to the patient, then administering anti-PD-1 therapy to the patient in an amount sufficient to treat the cancer susceptible to anti-PD-1 therapy and to reduce immune related adverse effects (irAEs),
wherein a risk of failure to control cancer growth is lower following the depletion of the T_{FR} cells.

21. The method of claim 20, wherein the presence of T_{FR} cells is determined from a cancer tissue biopsy.

22. The method of claim 20, wherein the step of detecting is measuring mRNA, protein, or both.

23. The method of claim 20, wherein the step of detecting is measuring mRNA, protein, or both. In another aspect, the selective T_{FR} cell depleting therapy is at least one of, but not limited to, anti-IL1R2, anti-OX40, anti-TNFR2, anti-CCR8 antibodies or other targets specifically expressed or enriched on T_{FR} cells when compared to T_{REG} cells and other T cell populations.

24. The method of claim 20, wherein the selective T_{FR} cells depleting therapy is at least one of anti-CTLA-4, anti-IL1R2, anti-4-1BB, anti-ICOS, anti-GITR, anti-OX40, anti-TNFR2, or anti-CCR8 therapy.

25. The method of claim 20, wherein the T_{FR} cells are CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺ GITR⁺ T cells, or any combination thereof.

26. The method of claim 20, wherein the cancer is selected from a colorectal, a melanoma, a lung, a liver, a head and neck, and a breast cancer.

27. The method of claim 20, wherein the T_{FR} cells express or have a high level of expression one or more of the following markers: PD-1, BCL6, FOXP3, CXCR5, GITR, CTLA-4, 4-1BB, ICOS, Tox, Ki67, and TCF1.

28. The method of claim 20, wherein the presence of T_{FR} cells is determined by measuring the expression of two or more genes or proteins selected from Tnfrsf1b, Lag3, Tigit, Batf, Illr2, Ccr8, Pdcd1, Tox, CCR8, TNFRSF1B.

29. A method of determining if a patient has follicular regulatory T (T_{FR}) cells that will increase cancer growth or cause an immune-related adverse effect (irAE) when treated with anti-PD-1 therapy comprising:
obtaining a biological sample from a patient; and
detecting the T_{FR} cells in the biological sample by contacting the biological sample with antibodies that detect T cells expressing CD3⁺CD4⁺FOXP3⁺BCL6⁺ T cells, CD3⁺CD4⁺CXCR5⁺GITR⁺ T cells, CD3⁺CD4⁺CXCR5⁺FOXP3⁺BCL6⁺ T cells, CD3⁺CD4⁺CXCR5⁺FOXP3⁺ T cells, or CD3⁺CD4⁺CXCR5⁺BCL6⁺GITR⁺ T cells, or any combination thereof, when compared to a reference level generated for specific tumor types or a healthy patient, and detecting the T_{FR} cells in the biological sample, wherein if the patient has an increase in T_{FR} cells in the biological sample anti-PD-1 therapy will increase cancer growth or cause the irAE.

30. The method of claim 29, further comprising detecting the presence or a high level of expression of at least one of: GITR, CTLA-4, 4-1BB, ICOS, Tox, Ki67, or TCF1 on the T_{FR} cells.

31. The method of claim 29, wherein the step of detecting is measuring mRNA, protein, or both.

32. The method of claim 29, wherein the selective T_{FR} cell depleting therapy is at least one of: anti-IL1R2, anti-OX40, anti-TNFR2, anti-CCR8 antibodies or other targets specifically expressed or enriched on T_{FR} cells when compared to T_{REG} cells and other T cell populations

33. The method of claim 29, wherein a selective T_{FR} cell depleting therapy is at least one of anti-CTLA-4, anti-IL1R2, anti-4-1BB, anti-ICOS, anti-GITR, anti-OX40, anti-TNFR2, or anti-CCR8 therapy.

34. The method of claim 29, wherein the T_{FR} cells are not LIN⁻CD45⁺CD3⁺CD4⁺CXCR5⁻ FOXP3⁺BCL6⁻PD-1⁻ cells.

35. The method of claim 29, wherein the biological sample is a cancer tissue.

36. The method of claim 29, wherein the biological sample is selected from a colorectal, a melanoma, a lung, a liver, a head and neck, or a breast cancer tissue.

37. The method of claim 29, wherein the T_{FR} cells are PD-1^{high}.

38. A method of depleting follicular regulatory T cells (T_{FR}) cells without affecting regulatory T (T_{REGS}) cells, comprising:
treating a T cell population with a treatment that reduces or eliminates PD-1 expressing T_{FR} cells and co-administering anti-IL1R2 antibodies to protect T_{REGS}, in order to prevent or reduce immune related adverse events (irAEs).

39. The method of claim 38, wherein the irAE is stimulation of CD4 or CD8 T cell proliferation, T_{FR} cells infiltrating a tumor, or that reduces or abrogates the effectiveness of an anticancer therapy.

40. The method of claim 38, wherein the anticancer therapy is anti-PD-1 therapy of a cancer selected from colorectal, melanoma, lung, liver, head and neck, or breast cancer.

41. The method of claim 38, wherein the T_{FR} cells are PD-1^{high}.

42. The method of claim 38, wherein the selective elimination of T_{FR} cells is in vitro.

43. A method of depleting follicular regulatory T cells (T_{FR}) cells without affecting regulatory T (T_{REGS}) cells, comprising:
treating a patient with reagents that selectively eliminate (PD-1 expressing) T_{FR} cells without significantly affecting or depleting T_{REG} cells, in order to prevent or reduce the occurrence of immune related adverse events (irAEs).

44. The method of claim 43, wherein the anticancer therapy is anti-PD-1 therapy of a cancer selected from colorectal, melanoma, lung, liver, head and neck, or breast cancer.

45. The method of claim 43, wherein the T_{FR} cells are PD-1^{high}.

46. A method of reducing immune related adverse events (irAEs) comprising:
selectively depleting T_{FR} cells, but not all FOXP3-expressing (T_{regs}, T_{FR}, or both) cells, by specifically targeting T_{FR}-specific cells with at least one of anti-CTLA-4, anti-IL1R2, anti-4-1BB, anti-ICOS, anti-GITR, anti-OX40, or anti-CCR8 depletion.

47. The method of claim 46, wherein the irAE is stimulation of at least one of CD4 or CD8 T cell proliferation, T_{FR} infiltrating a tumor, or that reduces or abrogates the effectiveness of an anticancer therapy.

48. The method of claim 47, wherein the anticancer therapy is anti-PD-1 therapy of cancers selected from colorectal, melanoma, lung, liver, head and neck, or breast cancer.

49. The method of claim 48, wherein the T_{FR} cells are PD-1^{high}.
